(19) United States
(12) Patent Application Publication
(10) Pub. No.: US 2025/0049546 A1
(43) Pub. Date: Feb. 13, 2025

(54) IMPLANTS, FUNCTIONALIZED IMPLANT SURFACES AND RELATED SYSTEMS, DEVICES, COMPUTER PROGRAM PRODUCTS, AND METHODS

(71) Applicant: RTRS Investment, LLC, Dallas, TX (US)

(72) Inventors: Ruedger RUBBERT, Berlin (DE); Justin BERNSTEIN, Denver, CO (US); Sabine SCHULZ, Berlin (DE)

(73) Assignee: RTRS Investment, LLC, Dallas, TX (US)

(21) Appl. No.: 18/447,073

(22) Filed: Aug. 9, 2023

(51) Int. Cl. A61C 13/00; A61C 8/00; A61C 13/20

(52) U.S. Cl. CPC A61C 13/0018; A61C 8/0013; A61C 13/0004; A61C 13/20; A61C 2008/0046

(57) ABSTRACT

Various implementations of implants and implant surfaces for clinical rehabilitation or enhancement of a patient, related systems, and computer programs and methods for the design and manufacturing of implants are disclosed. A macroscale shape, a microscale surface texture, and a nanoscale surface topography are overlaid to increase, condition, and thereby functionalize an implant surface. A thin-film coating and/or laser interferometry is utilized to overlay on a machined implant substrate a nanoscale surface topography. Manufacturing the macroscale shape and the microscale texture may be performed with an ultrashort pulsed laser system in separate process steps. The design of a dental implant may be assisted by a self-learning computer program product, based on trained coupled shape models including, for example, mesh-based statistical shape and orientation models.

### BACKGROUND

### Field

This disclosure relates generally to implantable devices and to techniques to design and manufacture such devices and surfaces.

### Description of Related Art

In medicine, orthopedic and craniofacial laboratories and manufacturers are providing materials and medical devices to repair, augment and replace hard and soft tissue body parts. Physicians apply various clinical techniques utilizing the materials and medical devices for clinical rehabilitation or enhancement.

Dentistry is a branch of medicine that includes the restoration or replacement of teeth as embedded in the oral mucosa, and in the jawbone of a patient. Dental laboratories and industrial manufacturers are providing materials and medical devices to repair, augment and replace single or a plurality of teeth, or portions thereof. Dentists apply various clinical techniques utilizing the materials and medical devices for oral rehabilitation or enhancement. For instance, implantable devices and techniques to design and manufacture such devices may be used for clinical rehabilitation and enhancement of humans and other mammal species. Implants may be made of stainless steel, titanium, or ceramics, and may be shaped by primary shaping, by forming, by additive, by subtractive or by other manufacturing technologies and may require, for example, additional steps of being heat treated, sintered, or tempered.

The integration of implants surfaces in bone tissue is often referred to as osseointegration or osteointegration. The adhesion between bone and such implant surfaces is often referred to as bone bonding. Mucous membranes or skins line and cover parts of the human body. Implants that temporarily or continuously penetrate or cross the skin or the mucosa of a patient generally require a seal against infiltration of fluids, particulates, and bacteria from the outside into the body.

Established design and manufacturing modalities, techniques of shaping, and techniques of conditioning implants, as well as other medical devices, have deficiencies and limitations. Often, implant failure can be caused by lack of bone integration and/or insufficient soft tissue integration, caused by suboptimal surface functionalization in combination, for example, with overload of the implant during the healing phase. Further, most shaping, heat treatment, and surface conditioning technologies are complex, expensive, and require suboptimal step-by-step sequences with long machine lead times. After sintering, some ceramics are too hard to be machined efficiently by boring, milling, and turning using hardened steel, carbide, and/or polycrystalline diamond (PCD) tools. Fiber lasers leave in most cases a heat-affected zones that may damage desired material properties on the surface of a workpiece of interest. In addition, toxic chemical residues from cooling lubricants and/or etchants used in manufacturing may require extensive cleaning before medical devices can be considered safe for clinical use. Further, exposure of materials to aggressive chemicals for etching may cause disadvantageous modifications in the materials composition such as chemical depletion. The interactive design of custom-shaped portions of dental and other implants is time-consuming and requires skilled and trained technicians or engineers. All these effects, alone or in combination, may pose technical and/or economic problems.

It is with these observations in mind, among others, that various aspects of the present disclosure were conceived and developed.

### SUMMARY

Systems, methods, techniques, and devices presented herein address the foregoing problems by efficiently designing, shaping, conditioning, and functionalizing implants and implant surfaces to thereby enhance tissue integration, and/or tissue adhesion.

The technology disclosed herein is illustrated, for example, according to various aspects described below, including with reference to figures, FIGS. 1 to 14. Various examples of aspects of the technology disclosed herein are described below. These are provided as examples and do not limit the subject technology.

In some examples, a method to manufacture a customized dental implant for a pre-identified patient can include obtaining a proposed specification of a dental implant, the dental implant including an endosseous root portion and an occlusal facing portion configured to receive a dental prosthesis; obtaining a trained shape model, the trained shape model being descriptive of a statistical dental anatomy model, the statistical dental anatomy model including one or more statistical dental anatomy element shape models; obtaining a data set including one or more virtual representations of one or more dental anatomy elements of a dentition of a patient; forming an adapted shape model based on at least a portion of the trained shape model to fit the one or more virtual representations; and/or generating an updated specification by updating the proposed specification of the dental implant based at least in part on the adapted shape model.

In some instances, the method can include machining the dental implant at least partially based on the updated specification so that a surface of the dental implant at least partially correlates to the adapted shape model. The data set can include one or more two-dimensional images representative of the one or more dental anatomy elements of the dentition of the patient, a two-dimensional image of the one or more two-dimensional images can include a plurality of pixels having assigned gradual intensity values, and/or the two-dimensional image of the one or more two-dimensional images can be a video frame, a picture, a two-dimensional image generated by an intraoral scanner, a two-dimensional array, and/or an X-ray image. Furthermore, the data set can include one or more two-dimensional images representative of the one or more dental anatomy elements of the dentition of the patient, the one or more two-dimensional images can include a two-dimensional image, and/or the two-dimensional image of the one or more two-dimensional images can be a two-dimensional point cloud, a two-dimensional mesh, and/or a two-dimensional shape model. Additionally, the data set can include at least one three-dimensional image of the one or more dental anatomy elements of the dentition of the patient, the at least one three-dimensional image can include a plurality of voxels having assigned gradual intensity values, and/or the at least one three-dimensional image can be a CT, a cone beam CT, an MRI image, a three-dimensional X-ray, a frame of a dynamic three-dimensional model, a three-dimensional frame generated by an intraoral scanner, and/or a three-dimensional array. Moreover, the data set can include at least one three-dimensional image of the one or more dental anatomy elements of the dentition of the patient, and/or the at least one three-dimensional image can be a three-dimensional point cloud, a three-dimensional mesh, a three-dimensional surface scan, and/or a three-dimensional shape model.

In some examples, the one or more virtual representations of the one or more dental anatomy elements embodied in the data set can be unlabeled. Also, the adapted shape model can include at least one labeled virtual dental anatomy shape element, and/or the at least one labeled virtual dental anatomy shape element can include a numerical three-dimensional surface reconstruction of a corresponding dental anatomy element of the one or more dental anatomy elements. Furthermore, the one or more dental anatomy elements can include at least one of a tooth, a portion of the tooth, an alveolar socket, a portion of the alveolar socket, a gingival margin, and/or a portion of the gingival margin, and/or the at least one labeled virtual dental anatomy shape element can be associated with a reference to a label corresponding to a dental tooth numbering scheme. Additionally, the one or more statistical dental anatomy element shape models can include a plurality of trained constraint models of virtual statistical shape variabilities.

In some scenarios, a method can include an iterative numerical optimization process having one or more steps including: varying a virtual size of a virtual shape of a statistical dental anatomy element shape model of the one or more statistical dental anatomy element shape models within at least one virtual size constraint included in the plurality of trained constraint models of virtual statistical shape variabilities, varying a virtual local deformation of a virtual shape of the statistical dental anatomy element shape model within at least one virtual deformation constraint included in the plurality of trained constraint models of virtual statistical shape variabilities, and/or calculating a quality function. Furthermore, the updated specification can include at least one virtual three-dimensional design model representing at least a portion of the dental implant selected from a group including at least two of an abutment portion, an occlusal portion, a preparation post to receive a crown, a preparation post to receive a bridge, a preparation post to receive a prosthetic element, a transgingival portion, an implant neck, an endosseous portion, a root portion, an interface between the abutment portion and the endosseous portion, and/or a root-analogue portion. Additionally, the trained shape model can be a multi-dimensional parametrized model, the one or more statistical dental anatomy element shape models includes a statistical dental anatomy element shape model, and/or the statistical dental anatomy element shape model, or forming the adapted shape model, uses a numerical structure including at least one of a point distribution model, a principal component analysis, a vector array, a two-dimensional point cloud, a two-dimensional surface mesh, and/or a three-dimensional surface mesh.

In some instances, computer program can be stored or storable on a non-transitory processor-readable memory as executable instructions which, when executed by one or more processors, performs a computer process includes: obtaining a proposed specification of a dental implant, the dental implant including an endosseous root portion and an occlusal facing portion configured to receive a dental prosthesis; obtaining a trained shape model, the trained shape model being descriptive of a statistical dental anatomy model, the statistical dental anatomy model including one or more statistical dental anatomy element shape models; obtaining a data set including one or more virtual representations of one or more dental anatomy elements of a dentition of a patient; forming an adapted shape model based on at least a portion of the trained shape model fitting the one or more virtual representations; and/or generating an updated specification by updating the proposed specification of the dental implant based at least in part on the adapted shape model. In some examples, the computer process can include visualizing, at a display of an electronic device, an image output of the computer process. Also the computer process can include performing a method of teaching the trained shape model.

In some scenarios, a method to manufacture a customized dental implant for a pre-identified patient includes obtaining a proposed specification of a dental implant, the dental implant includes an endosseous root portion and an occlusal facing portion operable to receive a dental prosthesis; obtaining a trained coupled shape model, the trained coupled shape model being descriptive of a statistical dental anatomy model, the statistical dental anatomy model including a plurality of labeled statistical dental anatomy element shape models and a plurality of corresponding statistical dental anatomy element orientation models; obtaining a data set including one or more virtual representations of a plurality of dental anatomy elements of a dentition of a patient; forming an adapted coupled shape model based on at least a portion of the trained coupled shape model fitting the one or more virtual representations; and/or generating an updated specification by updating the proposed specification of the dental implant based at least in part on the adapted coupled shape model.

In some instances, the method can include machining the dental implant based at least in part on the updated specification so that a surface of the dental implant at least partially correlates to the adapted coupled shape model. The data set can include one or more two-dimensional images representative of the plurality of dental anatomy elements of the dentition of the patient, a two-dimensional image of the one or more two-dimensional images including a plurality of pixels having assigned gradual intensity values; and/or the two-dimensional image of the one or more two-dimensional images can be a video frame, a picture, a two-dimensional image generated by an intraoral scanner, a two-dimensional array, or an X-ray image. The data set can also include at least one two-dimensional image representative of the plurality of dental anatomy elements of the dentition of the patient, and/or the at least one two-dimensional image is a two-dimensional point cloud, a two-dimensional mesh, a two-dimensional shape model, and/or a two-dimensional coupled shape model. Additionally, the data set can include at least one three-dimensional image of the plurality of dental anatomy elements of the dentition of the patient, the at least one three-dimensional image can include a plurality of voxels having assigned gradual intensity values, and/or the at least one three-dimensional image can be a CT, a cone beam CT, an MRI image, a three-dimensional X-ray, a frame of a dynamic three-dimensional model, a three-dimensional frame generated by an intraoral scanner, and/or a three-dimensional array. Furthermore, the data set can include at least one three-dimensional image of the plurality of dental anatomy elements of the dentition of the patient, and/or the at least one three-dimensional image can be a three-dimensional point cloud, a three-dimensional mesh, a three-dimensional surface scan, and/or a three-dimensional coupled shape model. Also, the one or more virtual representations of the plurality of dental anatomy elements embodied in the data set can be unlabeled. The adapted coupled shape model can include at least one labeled virtual dental anatomy shape element, and/or the at least one labeled virtual dental anatomy shape element can include a numerical three-dimensional surface reconstruction of a corresponding dental anatomy element of the plurality of dental anatomy elements. The plurality of dental anatomy elements can include at least one of a tooth, a portion of the tooth, an alveolar socket, a portion of the alveolar socket, a gingival margin, or a portion of the gingival margin, and/or the at least one labeled virtual dental anatomy shape element is associated with a reference to a label corresponding to a dental tooth numbering scheme. At least one labeled statistical dental anatomy element shape model of the plurality of labeled statistical dental anatomy element shape models can include a plurality of trained shape constraint models of virtual statistical shape variabilities, and/or the plurality of corresponding statistical dental anatomy element orientation models can include a trained orientation constraint model of virtual statistical orientation variability.

In some examples, the forming can include performing an iterative numerical optimization process having one or more steps including: varying a virtual size of a virtual shape of a labeled statistical dental anatomy element shape model of the plurality of labeled statistical dental anatomy element shape models within at least one virtual size constraint included in the plurality of trained shape constraint models of virtual statistical shape variabilities, varying a virtual local deformation of a virtual shape of the labeled statistical dental anatomy element shape model within at least one virtual deformation constraint included in the plurality of trained shape constraint models of virtual statistical shape variabilities, and/or varying a virtual orientation of a virtual shape of the labeled statistical dental anatomy element shape model within at least one virtual orientation constraint included in the trained orientation constraint model of virtual statistical orientation variability, and/or the iterative numerical optimization process includes calculating a quality function. Furthermore, the updated specification can include at least one virtual three-dimensional design model representing at least a portion of the dental implant including at least one of an abutment portion, an occlusal portion, a preparation post to receive a crown, a preparation post to receive a bridge, a preparation post to receive a prosthetic element, a transgingival portion, an implant neck, an endosseous portion, a root portion, an interface between the abutment portion and the endosseous portion, and/or a root-analogue portion. Additionally, the trained coupled shape model can be a multi-dimensional parametrized model including at least one of a static two-dimensional model, a dynamic two-dimensional model, a three-dimensional model, and/or a dynamic three-dimensional model, and/or the plurality of labeled statistical dental anatomy element shape models, or forming adapted coupled shape model, uses at least one numerical structure being at least one of a point distribution model, a principal component analysis, a vector array, a two-dimensional point cloud, a two-dimensional surface mesh, and/or a three-dimensional surface mesh.

In some scenarios, a computer program can be stored or storable on a non-transitory processor-readable memory as executable instructions which, when executed by one or more processors, performs a computer process comprising: obtaining a proposed specification of a dental implant, the dental implant includes an endosseous root portion and an occlusal facing portion operable to receive a dental prosthesis; obtaining a trained coupled shape model, the trained coupled shape model being descriptive of a statistical dental anatomy model, the statistical dental anatomy model including a plurality of labeled statistical dental anatomy element shape models and a plurality of corresponding statistical dental anatomy element orientation models; obtaining a data set including one or more virtual representations of a plurality of dental anatomy elements of a dentition of a patient; forming an adapted coupled shape model based on at least a portion of the trained coupled shape model to fit the one or more virtual representations; and/or generating an updated specification by updating the proposed specification of the dental implant based at least in part on the adapted coupled shape model. In some instances, the computer process can include visualizing, at display of an electronic device, an image output of the computer process. The computer process can also include performing a method of teaching the trained coupled shape model.

In some examples, a method to teach a dental anatomy machine learning model includes obtaining one or more individual exemplary dental anatomy models descriptive of one or more individual exemplary virtual dental anatomy shape elements; obtaining a trainable or trained shape model, the trainable or trained shape model is descriptive of a statistical dental anatomy model, the statistical dental anatomy model includes one or more statistical dental anatomy element shape models; and/or generating an updated trained shape model by updating the trainable or trained shape model based at least in part on the one or more individual exemplary dental anatomy models. Additionally, the one or more statistical dental anatomy element shape models can include a plurality of corresponding trained constraint models of virtual statistical shape variabilities, and/or the updating of the trainable or trained shape model can include updating, for the one or more statistical dental anatomy element shape models, the plurality of corresponding trained constraint models of virtual statistical shape variabilities based at least in part on a shape variability of the one or more individual exemplary virtual dental anatomy shape elements.

In some instances, a method to teach a dental anatomy machine learning model includes obtaining one or more individual exemplary dental anatomy models descriptive of a plurality of individual exemplary labeled virtual dental anatomy shape elements and corresponding exemplary virtual relative orientations; obtaining a trainable or trained coupled shape model, the trainable or trained coupled shape model being descriptive of a statistical dental anatomy model, the statistical dental anatomy model including a plurality of labeled statistical dental anatomy element shape models and a plurality of corresponding statistical orientation models; and/or generating an updated trained coupled shape model by updating the trainable or trained coupled shape model based at least in part on the one or more individual exemplary dental anatomy models. Moreover, the plurality of labeled statistical dental anatomy element shape models can include a plurality of corresponding trained shape constraint models of virtual statistical shape variabilities, and/or the updating of the trainable or trained coupled shape model can include updating, for the plurality of labeled statistical dental anatomy element shape models, the plurality of corresponding trained shape constraint models of virtual statistical shape variabilities based at least in part on a shape variability of the plurality of individual exemplary labeled virtual dental anatomy shape elements. Additionally, a corresponding statistical orientation models of the plurality of corresponding statistical orientation models can include a trained orientation constraint model of virtual statistical orientation variability, and/or the updating of the trainable or trained coupled shape model can include updating, for a labeled statistical dental anatomy element shape model of the plurality of labeled statistical dental anatomy element shape models, the trained orientation constraint model of virtual statistical orientation variability based at least in part on an orientation variability of the plurality of corresponding statistical orientation models.

Additional aspects, advantages, and/or utilities of the presently disclosed technology are set forth in part in the description that follows and, in part, will be apparent from the description, or may be learned by practice of the presently disclosed technology.

### BRIEF DESCRIPTION OF THE DRAWINGS

So that the manner in which the features, advantages, and objects of the technology, as well as others which will become apparent, are attained, and can be understood in more detail, more particular description of the technology briefly summarized above may be had by reference to the embodiments thereof which are illustrated in the appended drawings that form a part of this specification. It is to be noted, however, that the drawings illustrate only certain embodiments of the disclosed technology and are therefore not to be considered limiting of its scope as the disclosed technology may admit to other equally effective embodiments.

FIG. 1 shows an illustration of exemplary embodiments of dental implants, in accordance with one or more embodiments of the presently disclosed technology, which are combinable with any other embodiment disclosed herein.

FIG. 2 shows an illustration of exemplary embodiments of functionalized implant surfaces, in accordance with one or more embodiments of the presently disclosed technology, which are combinable with any other embodiment disclosed herein.

FIG. 3 shows an illustration of an exemplary embodiment of a porous implant surface, in accordance with one or more embodiments of the presently disclosed technology, which are combinable with any other embodiment disclosed herein.

FIG. 4 shows an illustration of an exemplary embodiment of a plurality of laser ablation patches, in accordance with one or more embodiments of the presently disclosed technology, which are combinable with any other embodiment disclosed herein.

FIG. 5 shows an illustration of an exemplary embodiment of a laser system, in accordance with one or more embodiments of the presently disclosed technology, which are combinable with any other embodiment disclosed herein.

FIG. 6 shows an illustration of an exemplary embodiment of a laser ablation process, in accordance with one or more embodiments of the presently disclosed technology, which are combinable with any other embodiment disclosed herein.

FIG. 7 shows an illustration of an exemplary embodiment of a 5-axis machine, in accordance with one or more embodiments of the presently disclosed technology, which are combinable with any other embodiment disclosed herein.

FIG. 8 shows an illustration of exemplary embodiments of a laser spallation process, in accordance with one or more embodiments of the presently disclosed technology, which are combinable with any other embodiment disclosed herein.

FIG. 9 shows an illustration of exemplary embodiments of a laser ablation process based on laser interferometry, in accordance with one or more embodiments of the presently disclosed technology, which are combinable with any other embodiment disclosed herein.

FIG. 10 shows a schematic block diagram illustrating an exemplary embodiment of an implant treatment and related information technology (IT) and other systems configuration and method steps in accordance with one or more embodiments of the presently disclosed technology, which are combinable with any other embodiment disclosed herein.

FIG. 11 shows an illustration of exemplary embodiments of a virtual shape adaptation process, in accordance with one or more embodiments of the presently disclosed technology which are combinable with any other embodiment disclosed herein.

FIG. 12 shows a schematic block diagram illustrating an exemplary embodiment of a neuronal network and a principal data flow in accordance with one or more embodiments of the presently disclosed technology, which are combinable with any other embodiment disclosed herein.

FIG. 13 shows an illustration of an exemplary embodiment of a flow diagram for a plurality of method steps for deriving or modifying iteratively an adapted coupled shape model descriptive of a dental anatomy in accordance with one or more embodiments of the presently disclosed technology which are combinable with any other embodiment disclosed herein.

FIG. 14 shows an illustration of an exemplary embodiment of a flow diagram for a plurality of method steps for shaping and functionalizing an implant in accordance with one or more embodiments of the presently disclosed technology, which are combinable with any other embodiment disclosed herein.

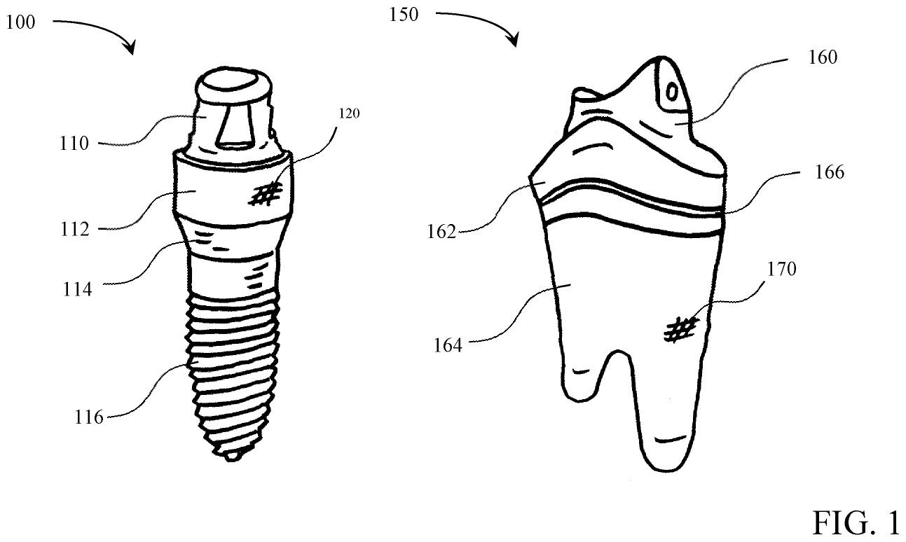

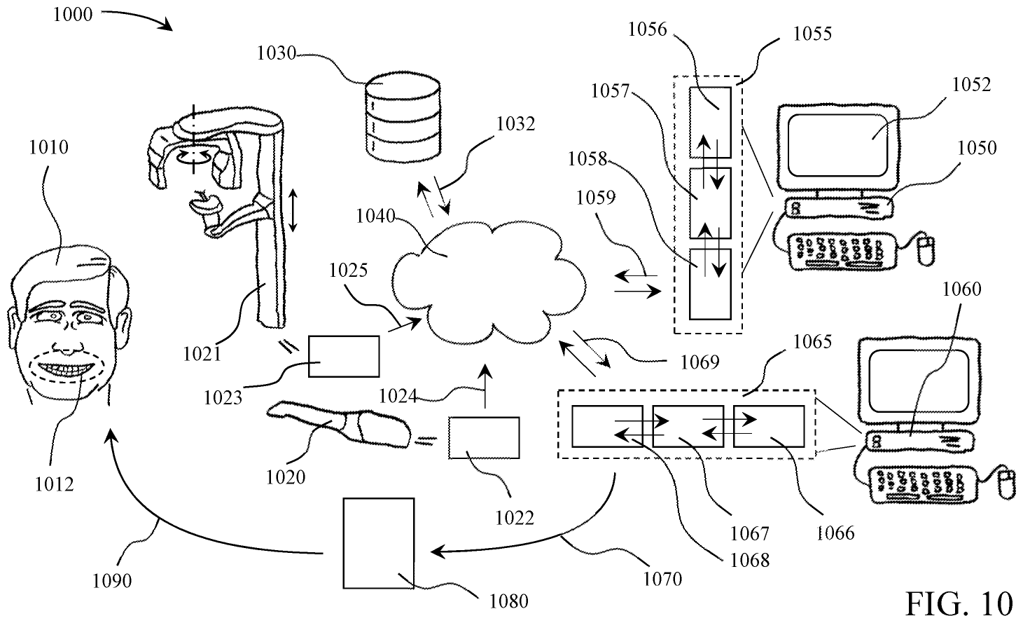

It should be noted that the first digit of a three-number numeral representing an element in the drawings refers to the number of the respective FIGS. 1 to 9. It should be noted that the first two digits of a four-number numeral representing an element in the drawings refer to the number of the respective FIGS. 10 to 14.

### DETAILED DESCRIPTION

The presently disclosed technology will now be described more fully hereinafter with reference to the accompanying drawings, which illustrate embodiments of the presently disclosed technology. This technology may, however, be embodied in many different forms and should not be construed as limited to the illustrated embodiments set forth herein. Rather, these embodiments are provided so that this disclosure will be thorough and complete, and will fully convey the scope of the presently disclosed technology to those skilled in the art. Like numbers refer to like elements throughout. The different numbering of identical or similar components and/or prime notation, if used, indicates similar elements in alternative embodiments and/or configurations.

The system(s) and method(s) provided by the various embodiments of the present technology comprise several independent novel and nonobvious features providing substantial improvements. The greatest benefit can be achieved in the field of implants, including but not limited to, dental implants and the computer-aided design and manufacturing of such implants.

One or more of the objects and/or features described in this, the preceding, and the following paragraph(s) may be combined in any combination and in no or in any order. One or more of the method, process and/or function steps described in this, the preceding and the following paragraph(s) may be combined in any combination and in no or in any order. One or more of the objects described in this, the preceding and the following paragraph(s) may be configured to carry out one or more of the method, process and/or function steps disclosed in this, the preceding and the following paragraph(s) in any combination and in no or in any order.

As discussed in greater detail below, medical devices that are integrated in the body of a patient, referred to as implants, may be shaped and/or conditioned to integrate with the embedding soft and hard tissue. Surfaces of implants can be conditioned or functionalized to adhere or bond to adjacent bone, mucosa, skin, and other tissues.

In some examples, clinical techniques disclosed herein can be applied by physicians and dentists and can include the individual adaptation and customization of a shape or shapes of materials and medical devices to fit the anatomical shape or shapes the patient presents. The clinical techniques may also include the individual adaptation of a shape or shapes of an anatomy the patient presents to fit dimensional shapes of the medical devices utilized. Laboratories and industrial manufacturers may receive medical imaging data representing, for example, a shape of a patient's specific anatomy and design and manufacture custom-shaped medical devices responsive to the imaging data, so that, for example, a shape of a medical device corresponds specifically to a shape of the patient's anatomy.

Furthermore, custom-shaped portions of implants may be designed by interactive CAD/CAM computer program products and manufactured by CNC machinery. CAD is an abbreviation for computer aided design, CAM for computer aided manufacturing and CNC for computerized numerical control.

FIG. 1 shows an illustration of exemplary embodiments of dental implants, in accordance with one or more embodiments of the presently disclosed technology, which are combinable with any other embodiment disclosed herein.

In one or more exemplary embodiments of the present technology, an implant, for example, a dental implant 100, and 150 comprises an endosseous portion 116, and 164. The dental implant 100, and 150 may further include an occlusal facing portion 110, and 160, a transmucosal portion 112, and 162, a micro-grooved portion 114, and 166, and/or one or more functionalized surfaces 120, and 170, or any combination thereof. An implant, for example the dental implant 100, and 150 may be made of a single material. An implant, for example, the dental implant 100, and 150 may comprise of multiple materials in combination with one another. Said single material or said multiple materials may include, for example, ceramic materials, for example, silicon nitride, zirconia, alumina, alumina toughed zirconia (ATZ), or zirconia toughed alumina (ZTA), metal materials, for example, titanium, titanium alloys, or stainless-steel alloys, polymeric materials, for example, Polyurethane ether ketone (PEEK) or Polymethylmethacrylate (PMMA), or organic materials, for example, collagen and osteoblast containing matrices. A dental implant 100 may have a main diameter between 2.5 mm and 14 mm, for example about 4.5 mm, and may have a length between 8 mm and 28 mm, for example about 16 mm. A dental implant 150 may have a dimensional envelope between 2.5 mm×2.5 mm×8 mm and 14 mm×16 mm×28 mm, for example about 4 mm×6 mm×18 mm.

A shaping of any portion of an implant, for example, of a dental implant 100, and 150 or of any of the material or materials may include a process of subtractive shaping, a process of additive shaping, a process of shape forming, and/or a rapid prototyping process. The occlusal facing portion 110, and 160 may be operationally formed and/or otherwise configured to receive a prosthesis or a prosthetic element, for example, a cap, a crown, a bridge, a denture, or a segment of a denture. A shape of the occlusal facing portion 110, and 160 may form one side of a form-locking fit with the prosthesis or the prosthetic element. The occlusal facing portion 110 may be of generic shape to thereby be available as a generically-shaped or non-customized occlusal facing portion 110. The occlusal facing portion 160 may be of a shape prescribed or otherwise specified for a pre-identified patient to thereby be available as a patient-individual or custom-shaped occlusal facing portion 160. The custom-shaped occlusal facing portion 160 may, at least partially, match or otherwise correlate to a crown shape of a tooth of the pre-identified patient, for example, a crown shape of a specific tooth of the pre-identified patient, designated for extraction, to be replaced with the dental implant 150. The endosseous portion 116 of the implant, for example, the dental implant 100, may be of generic shape to thereby be available as a generically-shaped or non-customized endosseous portion 110. The endosseous portion 164 of the implant, for example, the dental implant 150, may be of a shape prescribed or otherwise specified for a pre-identified patient to thereby be available as a patient-individual or custom-shaped endosseous portion 164. The custom-shaped endosseous portion 164 may, at least partially, match or otherwise correlate to a root shape of a tooth of the pre-identified patient, for example, a root shape of a specific tooth of the pre-identified patient, designated for extraction, to be replaced with the dental implant 150. Said custom-shaped dental implant 150 is herein also referred to as a “root-analogue” dental implant 150. A root-analogue dental implant 150 may be shaped, or otherwise configured to replace a single-rooted tooth, for example a central incisor, a lateral incisor, a canine, or a premolar of an upper or lower jaw of the pre-identified patient. A root-analogue dental implant 150 may be shaped, or otherwise configured to replace a multi-rooted tooth, for example a premolar, a molar, or a wisdom tooth of an upper or lower jaw of the pre-identified patient.

The endosseous portion 116 may be threaded to form a screw, any operationally, or otherwise configured to be clinically screwed into the jawbone of a patient. The endosseous portion 116 may be operationally shaped or otherwise configured to be clinically inserted by a mainly non-rotational movement, for example being tapped-in, to form a press-fit with a bony implant bed. A generically-shaped or a custom-shaped screw-in or tap-in dental implant 100, and 150 is herein also referred to as a “root-form” dental implant 100, and 150. The endosseous portion 164 may be operationally shaped or otherwise configured to be clinically inserted by a mainly non-rotational movement, for example being tapped-in, to form a press-fit with an alveolar socket of the pre-identified patient and/or the bony implant bed.

The endosseous portion 116, and 164 may be operationally shaped and/or otherwise configured to, when clinically inserted, to integrate mainly with the adjacent jawbone of the patient, or the pre-identified patient. The endosseous portion 116, and 164 may be operationally shaped and/or otherwise configured to, when clinically inserted, to integrate mainly with a periodontal structure of the patient. The transmucosal portion 112 may be of generic shape to thereby be available as a generically-shaped or non-customized transmucosal portion 112. The transmucosal portion 162 may be of a shape prescribed or otherwise specified for a pre-identified patient to thereby be available as a patient-individual or custom-shaped transmucosal portion 162. The custom-shaped transmucosal portion 162 may, at least partially, match or otherwise correlate to a shape of a tooth, a shape of a gingival portion, a shape of a gingival margin, or a shape of a bone crest of a tooth or a shape of an alveolar socket of the pre-identified patient, for example, an anatomical shape correlating to a specific tooth of the pre-identified patient, designated for extraction, to be replaced with the dental implant 150. A design of a custom-shaped transmucosal portion 162 having a virtual shape correlating to a specific tooth of a pre-identified patient may have a virtual cross-section including at least partially a virtual nearly straight or concave outline. The design of the custom-shaped transmucosal portion 162 having a virtual nearly straight or concave outline may be updated so that the virtual nearly straight or concave cross-sectional outline may form a convex cross-sectional outline. The updated design of the custom-shaped transmucosal portion 162 may have predominately a cross-sectional convex outline devoid of concave or straight outline segments. A cross-section of a custom-shaped transmucosal portion 162 manufactured responsive to the updated design may correlate at least partially with an anatomical shape correlating to a specific tooth of the pre-identified patient, designated for extraction, to be replaced with the dental implant 150, for example, a shape of the specific tooth, a shape of a gingival portion adjacent the specific tooth, a shape of a gingival margin adjacent the specific tooth, or a shape of a bone crest adjacent the specific tooth, or a shape of an alveolar socket adjacent the specific tooth.

Further, a custom-shaped or generically-shaped endosseous portion 116, and 164 may be combined with a custom-shaped or generically-shaped transmucosal portion 112, and 162, in any combination, and vice versa. A custom-shaped or generically-shaped endosseous portion 116, and 164 may be combined with a custom-shaped or generically-shaped occlusal facing portion 110, and 160, in any combination, and vice versa. A custom-shaped or generically-shaped transmucosal portion 112, and 162 may be combined with a custom-shaped or generically-shaped occlusal facing portion 110, and 160, in any combination, and vice versa.

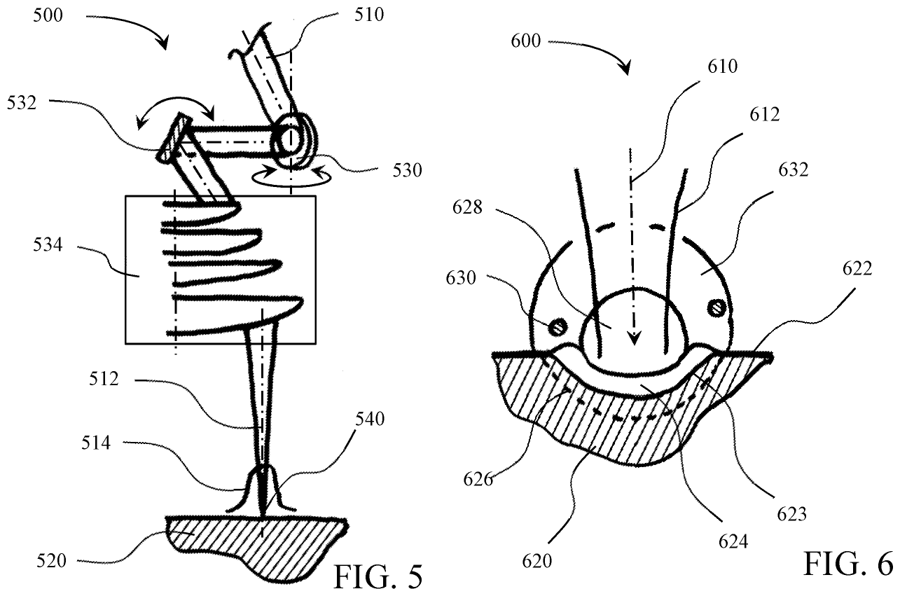

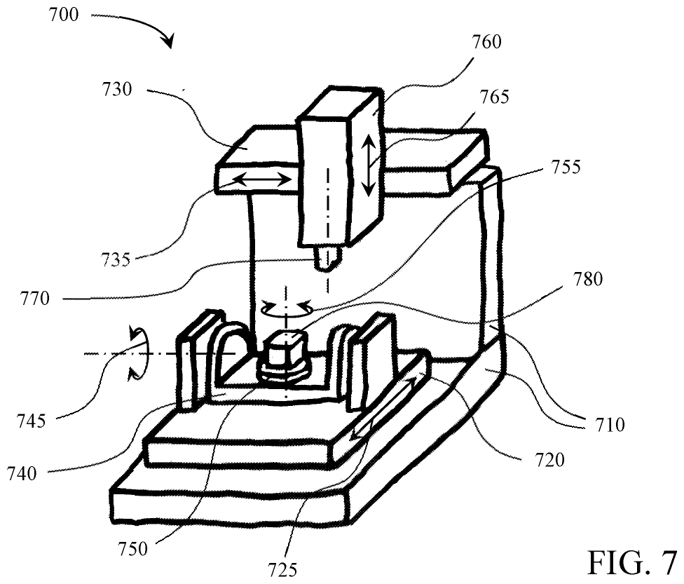

In this context and throughout this disclosure, the terms “macroscale shape” and “macroscale net or near net shape” and derivative or similar words shall be understood as being generic to all possible meanings supported by the specification and by the words itself; the meaning shall, however, include herein, without limiting the foregoing and unless the context requires otherwise, a spatial macroscale surface, form or contour of an outside of a body of solid material in its final or close to final spatial extension. The term “macroscale” in this context shall be understood that the dimensional extension(s) of the defining surface, form or contour elements are predominately greater than about 100 micrometers. By way of example, and not limitation, macroscale form or contour elements in this context may be cylindrical, even, planar, straight, parallel, angled, conical, spherical, pointy, sharp, helical or of spatial free form. The macroscale form or contour elements may be superimposed by finer surface structures such as one or more microscale textures or one or more nanoscale topographies. In this context and throughout this disclosure, the term “subtractive shaping” and derivative or similar words shall be understood as being generic to all possible meanings supported by the specification and by the words itself; the meaning shall, however, include herein, without limiting the foregoing and unless the context requires otherwise, shall include but shall not be limited to CNC grinding, CNC turning, CNC laser or water cutting or shaping, CNC milling technologies, femtosecond or other laser ablation technologies, acid etching, ultrasonic grinding, and/or other machining and finishing technologies. In this context and throughout this disclosure, the term “laser ablation” and derivative or similar words shall be understood as being generic to all possible meanings supported by the specification and by the words itself; the meaning shall, however, include herein, without limiting the foregoing and unless the context requires otherwise, a process of removing material from a solid body of material by irradiating it with a focused laser beam to thereby, as a result of the laser/material interaction, melt and remove and/or sublimate the matter. Laser ablation may be completed using a manufacturing system operable to be responsive to computer numerical control (CNC) instructions, using, for example, machine stages and/or galvanometer mirrors to deflect and/or focus a single laser beam. See FIGS. 5 and 7, for example. In this context and throughout this disclosure, the term “additive shaping” and derivative or similar words shall be understood as being generic to all possible meanings supported by the specification and by the words itself; the meaning shall, however, include herein, without limiting the foregoing and unless the context requires otherwise, shall include but shall not be limited to selective laser melting, selective laser sintering, stereo-lithography, 3D printing or depositing of wax, wax-bound powders, adhesive-bound powders, or slurries, chemical vapor deposition (CVD), initiated chemical vapor deposition (iCVD), and substrate material addition such as, but not limited to, natural or denaturized mammalian dentin.

In an exemplary embodiment, unless the context requires otherwise, combinable with any other embodiment disclosed herein, the dental implant 100, and 150 may form a one-piece implant or may form a two-piece assembly. The occlusal portion of the two-piece dental implant 100, and 150 may herein also be referred to as an “abutment”. The abutment may include the occlusal facing portion 110, and 160, and the transmucosal portion 112, and 162, or, alternatively, just the occlusal facing portion 110, and 160. The apical portion of the two-piece dental implant 100, and 150 may include the endosseous portion 116, and 164, and the transmucosal portion 112, and 162, or, alternatively, just the endosseous portion 116, and 164. The abutment and the apical portion of the of the two-piece dental implant 100, and 150 may be connected mechanically, for example, by directly screwing into each other, or through an additional screw. The abutment and the apical portion of the two-piece dental implant 100, and 150 may be made of the same or similar material or may be made of different materials. The abutment and the apical portion of the two-piece dental implant 100, and 150 may be permanently connected or fused, or these two portions may be detachable. The abutment and the apical portion of the two-piece dental implant 100, and 150 may be pre-assembled by a medical device manufacturer or clinically by dentist or dental specialist. The abutment and the apical portion of the dental implant 100, and 150 may be connected adhesively by a substrate material. The substrate material may be glass-based, cement-based, or resin-based. The abutment and the apical portion of the dental implant 100, and 150 may be connected by glass welding or by cyanoacrylate gluing. The one-piece dental implant 100, and 150 may be shaped from a solid workpiece or formed by injection molding. The dental implants 100 and 150 may include an integral root portion comprising of portions 116, or 164, respectively, and an integral abutment portion, which may comprise of portions 110, and 112, or 160, and 162, respectively.

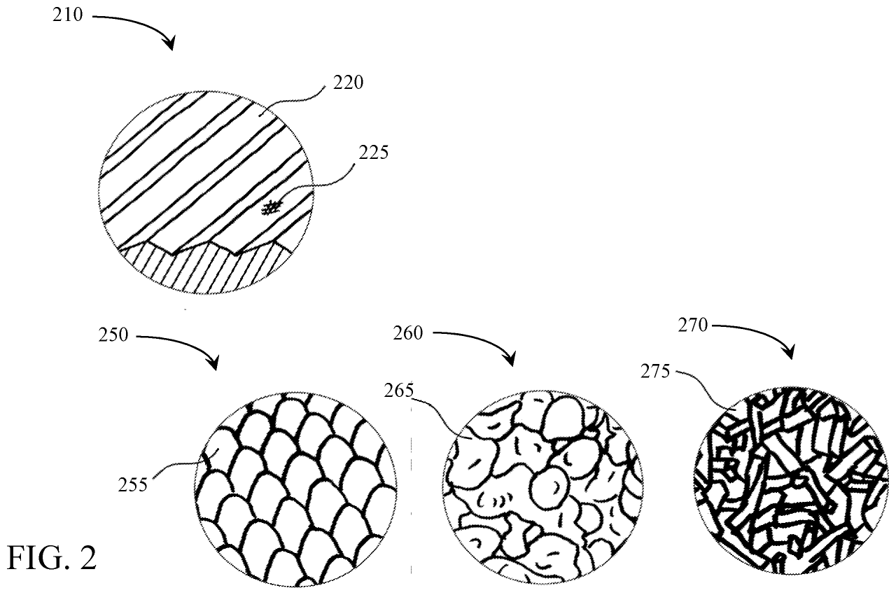

In an exemplary embodiment, unless the context requires otherwise, combinable with any other embodiment disclosed herein, the surface of the dental implant 100, and 150 may comprise, at any location, a functionalized surface 114, 120, 166, and 170. A functionalized surface 114, 120, 166, and 170 may comprise a microscale texture and/or nanoscale topography. A microscale texture may be formed onto a macroscale shape of an implant, for example, a dental implant 100, and 150. A nanoscale topography may be formed onto a macroscale shape of an implant, for example, a dental implant 100, and 150. A nanoscale topography may be formed onto a microscale texture of an implant, for example, a dental implant 100, and 150. A microscale texture, e.g., 114, 120, 166, and 170 may include surfaces 210, 322, and 622 as shown in and/or described with respect to FIGS. 2, 3, and 6, and other figures herein. A nanoscale topography, e.g., 114, 120, 166, and 170 may include surfaces 250, 260, 270, 322, and 622 as shown in and/or described with respect to FIGS. 2, 3, and 6, and other figures herein. In this context and throughout this disclosure, the term “formed onto” and derivative or similar words shall be understood as being generic to all possible meanings supported by the specification and by the words itself; the meaning shall, however, include herein, without limiting the foregoing and unless the context requires otherwise, an additional finer surface form actively superimposed on a previously existing coarser surface, or a finer surface form present on a coarser surface structure. In this context and throughout this disclosure, the term “microscale texture” or “microscale surface texture” and derivative or similar words shall be understood as being generic to all possible meanings supported by the specification and by the words itself; the meaning shall, however, include herein, without limiting the foregoing and unless the context requires otherwise, a spatial microscale surface configuration or structure of an outside of a physical body including its relief. The term “microscale” in this context shall be understood that the dimensional extension(s) of the defining surface relief elements or structures are predominately greater than about 2 micrometers and smaller than about 100 micrometers. By way of example, and not limitation, microscale surface texture structures or relief elements in this context may represent an engraving, a lithography pattern, a surface roughness, a sandblasted surface, an etched surface, undercuts, or a porous surface. The microscale texture may be superimposed by finer surface structures such as one or more nanoscale topographies. In this context and throughout this disclosure, the term “nanoscale topography” or “nanoscale surface topography” and derivative or similar words shall be understood as being generic to all possible meanings supported by the specification and by the words itself; the meaning shall, however, include herein, without limiting the foregoing and unless the context requires otherwise, a spatial nanoscale surface characteristics or structures of an outside of a physical body. The term “nanoscale” in this context shall be understood that the dimensional extension(s) of the defining surface characteristics or structures are predominately smaller than about 2 micrometers. By way of example, and not limitation, nanoscale surface characteristics or structural elements in this context may represent an engraving, a lithography pattern, an etched surface, a porous surface, an ablation pattern, a spallation pattern, a grain structure, or a structure of a coating. A machining of a macroscale shape, of a micro-grooved portion 114, and 166, of a functionalized surface 120, and 170, of a microscale texture, and/or a nanoscale texture may use a process of subtractive shaping, a process of additive shaping, a process of shape forming, and/or a rapid prototyping process. The micro-grooved portion 114, and 166 and/or the endosseous portion 116 may have a thread or threads. The micro-grooved portion 114, and 166 and/or the endosseous portion 116 may have threads of equal or different pitches and/or sizes.

In an exemplary embodiment, unless the context requires otherwise, combinable with any other embodiment disclosed herein, an implants, for example, an implant, for example, a dental implant 100 and 150 may be operational or otherwise configured for integration into or with specific types of mammalian tissue. A transmucosal portion 112, and 162 may be operational or otherwise configured for integration into or with mammalian mucosa. An implant portions 112 and 162 could be alternatively operational or otherwise configured as part of a trans-cutaneous implant to be integrated into mammalian cutaneous or subcutaneous mammalian tissue. A portion 116 and 164 of an implant, for example a dental implant 100 and 150 may be operational or otherwise configured to be integrated into mammalian bone and/or periodontal tissue. In this or a similar context throughout this disclosure, the term “configured” and derivative or similar words shall be understood as being generic to all possible meanings supported by the specification and by the words itself; the meaning shall, however, include herein, without limiting the foregoing and unless the context requires otherwise, shaping of portions, and/or functionalization of macroscale shape, of a microscale texture, and/or a nanoscale topography to optimize physical fit, and/or cell integration, into or with a correlating anatomical structure or tissue type.

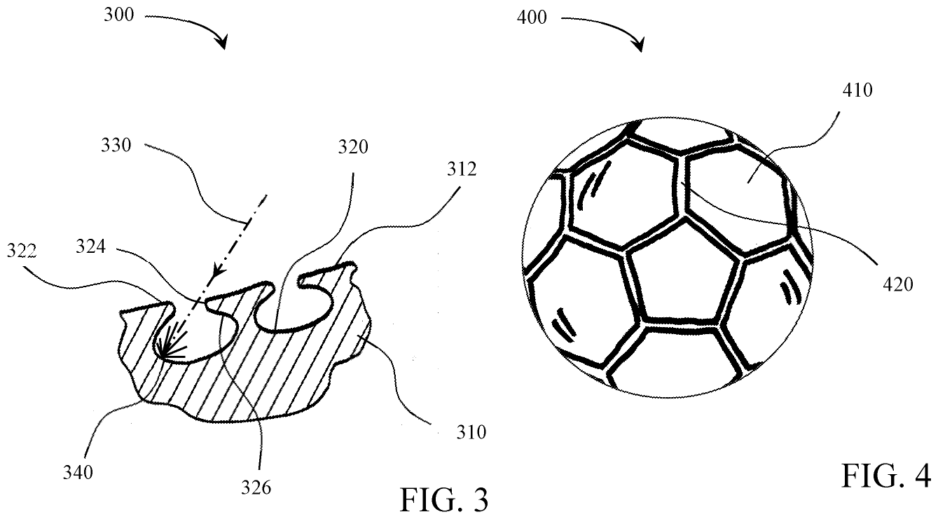

Without limiting the foregoing and unless the context requires otherwise, all features or other elements as disclosed herein with respect to FIG. 1 shall be deemed herewith disclosed in any combination with each other, in any partial combination with each other, and in any order; and shall be deemed herewith disclosed in combination with all features or other elements as disclosed herein with respect to FIGS. 2 to 14 in any combination, in any partial combination, and in any order. Without limiting the foregoing and unless the context requires otherwise, the dental implants 100, 150 referenced with respect to FIG. 1 may represent the same or similar dental implant, e.g., 300 or 1090, or the same or similar like elements, respectively, as shown in and/or described with respect to FIGS. 3, 10, 14, and other figures herein, respectively, and vice versa. Without limiting the foregoing and unless the context requires otherwise, the implant surfaces 112, 114, 116, 120, 160, 161, 164, 166, 170 referenced with respect to FIG. 1 may represent the same or similar surfaces, e.g., 210, 400, 826, 868, or 924, or like elements as shown in and/or described with respect to FIGS. 2 to 4, 8, 9, and other figures herein, respectively. Without limiting the foregoing and unless the context requires otherwise, the implant body 100 or 150 referenced with respect to FIG. 1 may represent the same or similar implant, e.g., 310, the same or similar workpiece, e.g., 520, 620, 755, 822, 922, or the same and similar like elements, respectively, as shown in and/or described with respect to FIGS. 3, 5 to 9, 14, and other figures herein, respectively, and vice versa. Without limiting the foregoing and unless the context requires otherwise, the mammalian cells, the mammalian cell variants, the mammalian tissue, and the mammalian tissue variants, as each referenced with respect to FIG. 1 may represent, the same or similar mammalian cells, the same or similar mammalian cell variants, the same or similar mammalian tissue, the same or similar mammalian tissue variants, or the same or similar like elements, respectively, as described and/or shown with respect to FIGS. 2, 3, 10, 14, and other figures herein, respectively, and vice versa. Without limiting the foregoing and unless the context requires otherwise, the manufacturing process, and the various method steps referenced with respect to FIG. 1 may represent the same or similar manufacturing process, e.g., 1080, or 1400, the various same or similar like method steps, e.g., 1410, 1420, 1430, 1440, 1450, or 1460, or the same or similar like elements, respectively, as described and/or shown with respect to FIGS. 2, 3, 10, 14, and other figures herein, respectively, and vice versa.

FIG. 2 shows an illustration of exemplary embodiments of functionalized implant surfaces, in accordance with one or more embodiments of the presently disclosed technology, and, unless the context requires otherwise, combinable with any other embodiment disclosed herein.

In one or more exemplary embodiments of the present technology, unless the context requires otherwise, combinable with any other embodiment disclosed herein, a functionalized surface 210 comprises a microscale surface texture, also referred to as microscale texture, 220. The microscale surface texture 220 may have grooves as depicted in FIG. 2. The grooves may have a hatch distance of 20 to 500 micrometers, for example, 200 micrometers. The profile of the cross-section of the grooves may have a height of 10 to 500 micrometers, for example 50 micrometer. The cross-section of the groove or grooves may have sharp edges, round edges, undercuts, or any combination thereof. The direction of the groove or grooves may be circumferential if the implant presents a substantially rotational symmetric extension. The groove or groves may be oriented mainly in line with a longitudinal axis of an extension of the implant, if present. The cross-section of the groove or grooves may be sawtooth-like, or trapezoidal-like. The groove or groves may be of a straight cut or intermitted. Circumferential groves may be closed or thread-like. Thread-like circumferential groves may be single-pitched or multi-pitched, also referred to as single-start or multi-start threads. Sets of such grooves of different directions may be superimposed or combined to form a hatch pattern or a cross pattern. The combination may be structured, deterministic or random-like. A hatch pattern 220 may be chess-board-like. In the process of designing a microscale surface texture 220, bionic or biologically inspired engineering principles may be employed. For example, a microscale surface texture 220 may comprise a profile of a fish-skin-like scale pattern. A fish scale pattern 220 may have a gradient of friction so that an insertion of an implant, for example, into human or other mammalian hard tissue is promoted while a movement of the implant through the tissue in a different, for example, opposite direction is obstructed or hindered. For example, a microscale surface texture 220 or a nanoscale surface 250 may comprise a plurality of holes, formed, for example, by a laser drilling process, including, for example, a percussion drilling process. A virtual model of a microscale surface texture 220 may be digitally superimposed, for example, digitally wrapped around a virtual model of a macroscale shape. A microscale surface texture 220 may comprise multiple patches of different orientations of the same structure or pattern or may comprise multiple patches of different structures or patterns. Such patches of microscale surface textures 220 may have a main dimension of 500 micrometers to 10 mm, for example 2 mm. The patches may have a triangular, rectangular, or hexagonal outline.

A microscale texture 210, and 220 may be formed onto a macroscale shape of an implant. A nanoscale surface topography 225, 255, 260, 265, 270, and 275 may be formed onto the microscale texture, otherwise understood as a “nanoscale functionalization”. A microscale texture 210, and 220 and/or a nanoscale topography may be formed through an additive shaping, subtractive shaping, shape forming, primary shaping, or rapid prototyping. For example, an injection molding tool may have a surface in the molding cavity that includes a microscale texture 210, and 220 and/or a nanoscale topography. An injecting molding process may form the macroscale shape and either microscale texture 210, and 220 or the nanoscale topography onto an implant through the injection molding process or other technologies of primary shaping. An injecting molding process may form the macroscale shape, the microscale texture 210, and 220 and the nanoscale topography. The injection molding tool cavity surface may be shaped, at least partially, by a laser ablation process. Injection molding may include metal injection molding or ceramic injection molding. A nanoscale topography 250, and 255 may be formed using a laser system, for example, in combination with the 5-axis machine. The laser system may be controlled through a set of computer numerical control (CNC) instructions. A nanoscale surface topography may be formed through a laser ablation process, a laser spallation process, or a laser ablation process based on laser interferometry. A nanoscale topography 250, may include interferometric laser ablation surface patterns 255. A process of direct laser interference patterning (DLIP) may form a periodic interference pattern 255 or laser-induced periodic surface structures (LIPSS) having a hatch distance of 10 nanometer to 10 micrometer, for example 1 micrometer. A nanoscale topography 260, and 265 may be formed through a thin-film coating process, applied, for example, by a chemical vapor deposition (CVD) or an initiated chemical vapor deposition (iCVD) process. The coating may comprise carbon, forming, for example, nano-crystallite diamond structures 265. nanocrystalline diamond (NCD) films 260, and 265 may contain grain sizes in the range of 50 nanometer to 20 micrometer, for example, 5 micrometer. The diamond crystallites may be rounded and not sharply facetted. A nanoscale topography 270, and 275 may be formed by a crystallite grain structure 275 of a substrate material, for example, of a ceramic material, for example, zirconia, alumina, or silicon nitride, Si₃N₄ 275. The dimensions of silicon nitrite grains 275 of longitudinal extension may have of cross-section dimension of 50 nanometers to 5 micrometer, for example 500 nanometer, and a length of 2 micrometer to 100 micrometer, for example, 10 micrometers. A nanoscale surface topography 225 may deterministically applied to the microscale texture 210, or a macroscale shape of an implant. In this context and throughout this disclosure, the term “deterministic” and derivative or similar words shall be understood as being generic to all possible meanings supported by the specification and by the words itself; the meaning shall, however, include herein, without limiting the foregoing and unless the context requires otherwise, surface elements being dimensionally formed, and placed responsive to engineering parameters. In contrast, non-deterministic or stochastic shaping processes, may be responsive to engineering parameters, however, the surfaces elements may be formed and may be placed by an impact of a stochastic interaction between, for example the distribution of a fluence of a focused laser beam with anisotropies of a substrate material. Non-deterministic laser processes are, for example, laser spallation processes.

Without limiting the foregoing and unless the context requires otherwise, all features or other elements as disclosed herein with respect to FIG. 2 shall be deemed herewith disclosed in any combination with each other, in any partial combination with each other, and in any order; and shall be deemed herewith disclosed in combination with all features or other elements as disclosed herein with respect to FIGS. 1, and 3 to 14 in any combination, in any partial combination, and in any order. Without limiting the foregoing and unless the context requires otherwise, the implant, the implant surface 210, 250, 260, and 270, the functionalized surface 210, the macroscale shape, the microscale surface texture 220, the nanoscale topography 225, 255, 260, 265, 270, and 275, the laser system, the 5-axis machine, the computer numerical control (CNC) instructions, the laser ablation process, the laser ablation process based on laser interferometry, the laser spallation process, the nano-crystallite diamond structures 265, and the crystallite grain structure 275 referenced with respect to FIG. 2 may represent the same or similar implant, e.g., 100, 150, or 1090, the same or similar surfaces implant surface, e.g., 112, 114, 116, 120, 160, 161, 164, 166, 170, 322, 400, 410, 622, 826, 866, 868, or 924, the same or similar functionalized surface, e.g., 114, 120, 166, 170, 322, 400, 410, 622, 826, 866, 868, or 924, the same or similar macroscale shape, e.g., 110, 112, 116, 160, 162, or 164, the same or similar microscale surface texture, e.g., 114, 120, 166, 170, 322, 400, 410, or 622, the same or similar nanoscale topography, e.g., 114, 120, 166, 170, 322, 400, 410, 622, 826, 866, 868, or 924, the same or similar laser system, e.g., 500, 770, or 1080, the same or similar 5-axis machine, e.g., 700, or 1080, the same or similar computer numerical control (CNC) instructions, the same or similar laser ablation process, e.g., 340, 540, 600, or 1080, the same or similar laser ablation process based on laser interferometry, e.g., 920, 940, or 960, the same or similar laser spallation process, e.g., 820, 840, or 860, the same or similar nano-crystallite diamond structures, the same or similar crystallite grain structure, or like elements as shown in and/or described with respect to FIGS. 1, 3, to 10, and other figures herein, respectively, and vice versa. Without limiting the foregoing and unless the context requires otherwise, the mammalian cells, the mammalian cell variants, the mammalian tissue, and the mammalian tissue variants, as each referenced with respect to FIG. 2 may represent, the same or similar mammalian cells, the same or similar mammalian cell variants, the same or similar mammalian tissue, the same or similar mammalian tissue variants, or the same or similar like elements, respectively, as described and/or shown with respect to FIGS. 1, 3, 10, 14, and other figures herein, respectively, and vice versa. Without limiting the foregoing and unless the context requires otherwise, the manufacturing process, and the various method steps referenced with respect to FIG. 2 may represent the same or similar manufacturing process, e.g., 1080, or 1400, the various same or similar like method steps, e.g., 1410, 1420, 1430, 1440, 1450, or 1460, or the same or similar like elements, respectively, as described and/or shown with respect to FIGS. 1, 3, 10, 14, and other figures herein, respectively, and vice versa. Without limiting the foregoing and unless the context requires otherwise, the virtual model, and the patch or patches referenced with respect to FIG. 2 may represent the same or similar virtual model, e.g., 1069, the same or similar patch or patches, e.g., 410, or the same or similar like elements, respectively, as described and/or shown with respect to FIGS. 4, 10, 11, 13, 14, and other figures herein, respectively, and vice versa.

FIG. 3 shows an illustration of an exemplary embodiment of a porous implant surface 300, in accordance with one or more embodiments of the presently disclosed technology, and, unless the context requires otherwise, combinable with any other embodiment disclosed herein.

In one or more exemplary embodiments of the presently disclosed technology, unless the context requires otherwise, combinable with any other embodiment disclosed herein, an implant body 310 comprises a porous implant surface 300 including a plurality of pores 320. The pores 320 may have a three-dimensional extension with a main dimension between about 1 micrometer and about 1 mm, for example, about 100 micrometer. Various manufacturing technologies may be used to form the pores 320. The pores 320 may be machined. By way of example, and not limitation, the pores 320 are shaped by a laser ablation process 340 using a laser beam 330. The laser beam 330 may be positioned in relation to the implant body 310 in a manufacturing system operable to be responsive to computer numerical control (CNC) instructions, using, for example, machine stages and/or galvanometer mirrors to deflect and/or focus a single laser beam 330 to thereby machine the shape of a pore 320. The shape of a pore 320 may correlate to a virtual shape of a design of a pore 320. The computer numerical control (CNC) instructions may be derived from a design of a pore 320. The machine stages and/or galvanometer mirrors may be operational or otherwise configured to form a multi-axes machine system so that the laser beam 330 can be positioned in relation to the implant body 310 so that the laser beam 330 penetrates the surface 312 of the implant body 310 through an opening 324 of the pore 320, forming the volume of the pore 320 having a dimensional extension greater than the dimensional extension of the opening 324 of the pore 320 to thereby form undercuts 326. Undercuts 326 in this context shall include surfaces of the pore 320 not visible when viewed mainly perpendicular to a local surface area 322 that includes the opening 324 of the pore 320. Multi-beam laser heads generating a plurality of laser beams 330 may be used to machine the shape of a plurality of pores 320 simultaneously. The pores 320 may be operational or otherwise configured to support the growth of mammalian cells. The pores 320 may be operational or otherwise configured to support the ingrowth of mammalian tissue, by way of example, not limitation bone, mucosa, natural dental cementum, periodontal ligament structures, cutaneous structures, and sub-cutaneous structures.

In one or more exemplary embodiments of the presently disclosed technology, unless the context requires otherwise, combinable with any other embodiment disclosed herein, a manufacturing process includes the method step of functionalizing a surface 312 of a dental implant 310 for integration into or with mammalian tissue, for example a periodontal ligament structure. The functionalized surface 312 may include a macroscale shape and a microscale texture formed on the macroscale shape. The functionalized surface 312 may include a plurality of pores 320 or a porous texture including a plurality of pores 320. A manufacturing process may include a method step, obtaining a specification of a dental implant 310. The specification may include requirements or instructions of the functionalized surface 312, for example, in accordance with one or more embodiments of the presently disclosed technology, unless the context requires otherwise, combinable with any other embodiment disclosed herein. A manufacturing process may include a method step, machining the porous texture using a laser ablation process, for example, as described above. A manufacturing process may include a method step, coating a surface 312. In this context or otherwise, a porous texture including a plurality of pores 320 may be filled, at least partially, for example to more than about 30%, with a biocompatible or a bio-active substance. The biocompatible or a bio-active substance may be resin or cementum based. The biocompatible or a bio-active substance may include a mineral or a mineral aggregate. The biocompatible or a bio-active substance may include at least traces of mammalian dentin. The mammalian dentin may be present as a powdery or grinded substance. The mammalian dentin may be of autologous origin. The mammalian dentin may be denaturalized. The dentin may cause natural dental cementum forming cells to migrate and/or not finally differentiated cells to differentiate to form natural dental cementum forming cells. The material of the implant may include a composite, a resin, a cementum, a mineral, a mineral aggregate, a dentin, or any combination thereof. The natural dental cementum forming cells may dispose a layer of natural dental cementum onto the surface 312 to thereby enhance or enable the integration of the dental implant 310 into or with a periodontal ligament structure.

Without limiting the foregoing and unless the context requires otherwise, all features or other elements as disclosed herein with respect to FIG. 3 shall be deemed herewith disclosed in any combination with each other, in any partial combination with each other, and in any order; and shall be deemed herewith disclosed in combination with all features or other elements as disclosed herein with respect to FIGS. 1, 2, and 4 to 14 in any combination, in any partial combination, and in any order. Without limiting the foregoing and unless the context requires otherwise, the dental implant 310 referenced with respect to FIG. 3 may represent the same or similar dental implant, e.g., 100, 150, or 1090, or the same or similar like elements, respectively, as shown in and/or described with respect to FIGS. 1, 10, 14, and other figures herein, respectively, and vice versa. Without limiting the foregoing and unless the context requires otherwise, the implant body 310 referenced with respect to FIG. 3 may represent the same or similar implant, e.g., 100, or 150, the same or similar workpiece, e.g., 520, 620, 755, 822, 922, or the same and similar like elements, respectively, as shown in and/or described with respect to FIGS. 1, 5, 6, 7, 8, 9, 14, and other figures herein, respectively, and vice versa. Without limiting the foregoing and unless the context requires otherwise, the porous implant surface 300 referenced with respect to FIG. 3 may be formed onto the surfaces, e.g., 110, 112, 114, 116, 120, 160, 161, 164, 166, 170, 210, 400, 410, 826, 868, or 924, or like elements as shown in and/or described with respect to FIGS. 1, 2, 4, 8, 9, and other figures herein, respectively. Without limiting the foregoing and unless the context requires otherwise, the laser ablation process 340, and the laser beam 330, as each referenced with respect to FIG. 3 may represent the same or similar laser ablation process, e.g., 540, or 600, the same or similar laser beam, e.g., 512, or 612, or the same and similar like elements, respectively, as shown in and/or described with respect to FIGS. 5, 6, and other figures herein, respectively, and vice versa. Without limiting the foregoing and unless the context requires otherwise, the manufacturing system, the computer numerical control (CNC) instructions, the machine stages, the galvanometer mirrors, the laser system, and the multi-axes machine system, as each referenced with respect to FIG. 3 may represent, the same or similar manufacturing system, e.g., 500, 700, and 1080, the same or similar computer numerical control (CNC) instructions, e.g., 1070, the same or similar machine stages, e.g., 720, 730, 740, 750, and 760, the same or similar galvanometer mirrors, e.g., 530, and 532, the same or similar laser system, e.g., 500, and 770, the same or similar multi-axes machine system, e.g., 500, and 700, respectively, or the same or similar like elements, respectively, as described and/or shown with respect to FIGS. 5 to 9, and other figures herein, respectively, and Without limiting the foregoing and unless the context requires otherwise, the vice versa. mammalian cells, the mammalian cell variants, the mammalian tissue, and the mammalian tissue variants, as each referenced with respect to FIG. 3 may represent, the same or similar mammalian cells, the same or similar mammalian cell variants, the same or similar mammalian tissue, the same or similar mammalian tissue variants, or the same or similar like elements, respectively, as described and/or shown with respect to FIGS. 1, 2, 10, 14, and other figures herein, respectively, and vice versa. Without limiting the foregoing and unless the context requires otherwise, the manufacturing process, and the various method steps referenced with respect to FIG. 3 may represent the same or similar manufacturing process, e.g., 1080, or 1400, the various same or similar like method steps, e.g., 1410, 1420, 1430, 1440, 1450, or 1460, or the same or similar like elements, respectively, as described and/or shown with respect to FIGS. 1, 2, 10, 14, and other figures herein, respectively, and vice versa.

FIG. 4 shows an illustration of an exemplary embodiment of a plurality of laser ablation patches, in accordance with one or more embodiments of the presently disclosed technology, and, unless the context requires otherwise, combinable with any other embodiment disclosed herein.

In one or more exemplary embodiments of the presently disclosed technology, unless the context requires otherwise, combinable with any other embodiment disclosed herein, a medical device manufacturing system comprises a laser system, and a workpiece, the laser system can generate a laser beam operational or can otherwise be configured for laser ablation processing of the workpiece. The medical device manufacturing system may further comprise a technical specification that identifies the workpiece as a semi-finished or finished product to become an implant. The implant includes in one or more exemplary embodiments of the presently disclosed technology, unless the context requires otherwise, combinable with any other embodiment disclosed herein, the implant surface 400. In this context and throughout this disclosure, the term “technical specification” and derivative or similar words shall be understood as being generic to all possible meanings supported by the specification and by the words itself; the meaning shall, however, include herein, without limiting the foregoing and unless the context requires otherwise, a full or partial description or identification of a designation, a design, a material composition, or a function, and any combination thereof. By way of example, and not limitation, the description may be made by direct or indirect reference and may include explicit or implicit elements. A technical specification may be paper-based or paperless, and may include a job order, a design, a drawing, a bill of materials, a manufacturing operating directive, a standard operating procedure, a material receipt, a delivery note, and/or the like. A design and/or manufacturing process may include the method steps to receive a technical specification of an implant, and to update the technical specification responsive to clinical imaging data.

The medical device manufacturing system may further comprise computer numerical control (CNC) data as computer-executable instructions embodied in one or more non-transitory processor-readable media. The computer numerical control (CNC) data as computer-executable instructions may be stored or storable on one or more non-transitory processor-readable memory. The computer numerical control (CNC) data as computer-executable instructions may be alternatively or additionally embodied in one or more non-transitory processor-readable computer data signals as computer-executable instructions. In this context and throughout this disclosure, the term “non-transitory processor-readable medium” and/or “statutory processor-readable medium” and derivative or similar words shall be understood herein as being generic to all possible meanings supported by the specification and by the words itself; provided, however, that the meaning shall include any non-transitory processor-readable memory or any non-transitory computer data signal except any non-patent-eligible subject matter as defined in the applicable jurisdiction by the then applicable law and case law as being not patentable. In this context and throughout this disclosure, the term “non-transitory processor-readable memory” and derivative or similar words shall be understood herein as being generic to all possible meanings supported by the specification and by the words itself; the meaning shall, however, include, without limiting the foregoing and unless the context requires otherwise, any medium that can store or transfer information, by way of example, and not limitation: any dynamic, static, read/write, read-only, sequential-access, location-addressable, file-addressable, and/or content-addressable data storage device; register memory, processor cache and RAM; any semiconductor memory device, ROM, flash memory, erasable ROM, EPROM, EEPROM, flash memory, or other solid state memory technology; magnetic memory, magnetic cassettes, magnetic tape, magnetic disk, or other magnetic data storage devices; floppy diskette, CD-ROM, digital versatile disks, DVD, HD-DVD, or BLU-RAY disc, optical disk, hard disk, MRAM, and/or like device. In this context and throughout this disclosure, the term “non-transitory computer data signal” and/or “statutory computer data signal” and derivative or similar words shall be understood herein as being generic to all possible meanings supported by the specification and by the words itself; provided, however, that the meaning shall include any computer data signal except any non-statutory subject matter as defined in the applicable jurisdiction by the then applicable law and case law as being not patentable. By way of example, and not limitation, non-transitory computer data signal shall include any physical, transferrable, and reproducible computer data signal. Such non-transitory computer data signals may include, by example and not limitation, data transmitted in blocks, followed by a check of the integrity of the receiver's data, so that, if there is a single bit error, the entire block must retransmit until the reproducibility has been guaranteed (e.g., “error correction”). In this context and throughout this disclosure, the term “computer data signal” and derivative or similar words shall be understood herein as being generic to all possible meanings supported by the specification and by the words itself; the meaning shall, however, include, without limiting the foregoing and unless the context requires otherwise: any signal that can propagate encoded information such as computer readable instructions, encoded logic, data, data structures, program modules, or other data over a transmission medium; other transport mechanisms, or delivery media such as a carrier wave; parallel or serial computer bus systems; electronic network channels; optical fibers; air, infrared, acoustic or electromagnetic paths; RF links; or other wired or wireless configurations. By way of example, and not limitation: computer networks such as the internet, intranet, LAN, serial or parallel bus systems, or otherwise; supported by network connectivity devices that may take the form of modems, modem banks, Ethernet cards, Universal Serial Bus (USB) interface cards, serial interfaces, token ring cards, fiber distributed data interface (FDDI) cards, wireless local area network (WLAN) cards, radio transceiver cards, and/or other like network connectivity devices. The network connectivity devices may provide wired communication links and/or wireless communication links. Wired communication links may be provided in accordance with Ethernet (IEEE 802.3), Internet protocol (IP), time division multiplex (TDM), data over cable service interface specification (DOCSIS), wavelength division multiplexing (WDM), and/or the like. Radio transceiver cards may provide wireless communication links using protocols such as code division multiple access (CDMA), Global System for Mobile Communications (GSM), LTE, WI-FI (IEEE 802.11), BLUETOOTH, ZIGBEE, narrowband Internet of things (NB IoT), near field communications (NFC), and radio frequency identity (RFID). The radio transceiver cards may promote radio communications using 5G, 5G New Radio, or 5G LTE radio communication protocols. Wireless connection may also proceed via satellite link (e.g., Starlink). These network connectivity devices may enable a processor or processors to communicate with the Internet or one or more intranets. With such a network connection, it is contemplated that a processor or processors might receive information from the network and/or might output information to the network. Such information, which is often represented as a sequence of instructions to be executed using a processor or processors, may be received from and/or outputted to the network, for example, in the form of a computer data signal embodied in a carrier wave.

The medical device manufacturing system may further comprise, for example, a first galvanometer operable to control a first mirror deflecting the laser beam in response to the computer numerical control data in a first direction; for example, a second galvanometer operable to control a second mirror deflecting the laser beam in response to the computer numerical control data in a second direction different than first direction; for example, an optical system operable to focus the laser beam and gain an intensity profile of the laser beam such that the intensity profile 514, as shown in and/or described in FIG. 5, exceeds at least partially an ablation threshold of the material of the workpiece, and an optical focus shifter (also referred to as a Z shifter), or a machine stage, operable to shift a focus of the laser beam in the direction of a main axis of the laser in relation to a surface of the workpiece, and a control unit. The control unit may include a computer and may be operable to control the laser system and the machine axes, for example, the first galvanometer, for example, the second galvanometer, for example, the focus shifter, and for example the machine stage or stages responsive to the computer numerical control (CNC) data, for example, as computer-executable instructions or otherwise.

In one or more exemplary embodiments of the presently disclosed technology, unless the context requires otherwise, combinable with any other embodiment disclosed herein, the workpiece, in a semi-finished stage of manufacturing, includes a first macroscale shape of spatial extension. After machining, using the laser ablation process as described above, the workpiece, in a final or near final stage of manufacturing, can include a second macroscale shape of spatial extension, deviating from the first macroscale shape of spatial extension. The differential volume between the first macroscale shape of spatial extension and the second macroscale shape of spatial extension can represent the material shaped of by the laser ablation process. The computer numerical control (CNC) data may be representative of a virtual differential laser ablation volume, that may be correlating to the differential volume. The virtual differential laser ablation volume may be correlating to a virtual semi-finished shape of the workpiece and correlating to a corresponding virtual shape model of an implant. The virtual semi-finished shape of the workpiece may have a virtual spatial macroscale extension that deviates from the virtual shape model of the implant. The virtual semi-finished shape of the workpiece may correlate to the first macroscale shape of spatial extension. The virtual shape model of the implant may correlate to the second macroscale shape of spatial extension of the workpiece. The virtual semi-finished shape of the workpiece may represent a generic shape of spatial macroscale extension, for example a cylindrical or a cubical shape. The virtual shape model of the implant may represent a custom-shape of spatial macroscale extension. The virtual shape model of the implant may correlate to an individual anatomical shape of a pre-identified patient. In this context and throughout this disclosure, the term “custom-shaped” and derivative or similar words shall be understood as being generic to all possible meanings supported by the specification and by the words itself; the meaning shall, however, include herein, without limiting the foregoing and unless the context requires otherwise, a reference to a shape of the object of interest of substantial spatial extension correlating to a corresponding spatial surface of a past, present, future, or projected state of a mammal anatomy, including, for example an anatomy of a pre-identified individual patient. In this context and throughout this disclosure, the term “patient” and derivative or similar words shall be understood as being generic to all possible meanings supported by the specification and by the words itself; the meaning shall, however, include herein, without limiting the foregoing and unless the context requires otherwise, a human subjected to a diagnostic exercise and/or undergoing a medical treatment, or subjected to a potential anticipated diagnostic exercise and/or a potential anticipated medical treatment. The virtual differential laser ablation volume may exceed about 5% of the volume of the workpiece or the virtual semi-finished shape of the workpiece. The medical device manufacturing system may further comprise an in-line metrology measurement instrument operational or otherwise configured to measure a shape of the workpiece. The virtual semi-finished shape of the workpiece may correlate to spatial metrology measurement data of a semi-finished shape of the workpiece.

In one or more exemplary embodiments of the presently disclosed technology, unless the context requires otherwise, combinable with any other embodiment disclosed herein, the medical device manufacturing system, including, for example, the laser system, the control unit, the first galvanometer, the second galvanometer, the optical system, and the focus shifter or the machine stage may be operational or otherwise configured to ablate a plurality of ablation layers, an ablation layer, also identified as a patch 410, of the plurality of ablation layers having a two-dimensional boundary or a three-dimensional boundary, and a layer thickness in the direction of the main axis of the laser beam within a spatial working range of the laser beam. The spatial working range may be defined by a range of the first galvanometer deflecting the laser beam, by a range of the second galvanometer deflecting the laser beam, and by a range of the optical focus shifter or the machine stage operable shifting the focus of the laser beam. The implant surface 400 is shaped by ablating a plurality of patches 410, as shown in and/or described with respect to FIG. 4. Each patch 410 layer may be ablated by a scanning pattern, where the focused laser beam and subsequently the laser ablation pool 600, as shown in and/or described with respect to FIG. 6, is deflected line by line, each line identified as a vector or a track, where the lines having a hatch distance of, for example, 2 to 50 micrometers. The medical device manufacturing system may be operational or otherwise configured to patch or map the plurality of patches 410, responsive to the computer numerical control data, onto a circumferential extension of the workpiece, thus machining a shape of the workpiece or the implant. The tracks may have an orientation in relation to main dimensions of the workpiece. In an exemplary embodiment of the presently disclosed technology, unless the context requires otherwise, combinable with any other embodiment disclosed herein, the medical device manufacturing system further comprises a rotary stage operational to be responsive to the computer numerical control data. The machine instructions included in the computer numerical control data may cause the medical device manufacturing system, including for example, the laser system, the control unit, a galvanometer, the optical system, and the rotary stage to position and orient the track or tracks of the laser ablation path parallel to a rotary axis of the rotary stage, and, for example, mainly perpendicular to a circumferential surface of a workpiece clamped onto the rotary stage. In this context, the galvanometer/mirror unit and the rotary stage may be operated in a coordinated manner so that a track movement of the laser ablation path is triggered by an incremental turn of the rotary stage. The laser may be operated in a pulse-on-demand mode, where a laser pulse is requested by a control unit, when the galvanometer/mirror unit and/or a machine stage, for example the rotary stage, has reached a position correlating to a predefined laser ablation track or vector. The incremental turn of the rotary stage may correlate to a hatch distance of laser tracks correlating to the diameter of the focused laser beam. In this context, the coordinated operation of an incremental turn triggering a track movement of the laser ablation path, may be repeated multiple times so that a circumferential layer of the workpiece is ablated. The boundaries of adjacent patches 420 may form a gap or an overlapping area. As the differential volume may be ablated not just by adjacent patches 410, but also layer-by-layer, with other words, in blankets of layers, where the ablation patches are virtually stacked atop of each other, the joints or gaps 420 between adjacent patches may be positioned in the virtual stack of patched at the same or similar position and orientation atop of each other, so that a cumulative joint or gap builds up, forming a groove 420 in the event that the adjacent patches 410 are overlapping, forming a wall 420, in the event that the adjacent patches are not overlapping but showing a gap. In an exemplary embodiment of the presently disclosed technology, unless the context requires otherwise, combinable with any other embodiment disclosed herein, the medical device manufacturing system may be operable to avoid build ups resulting from stacking joints or gaps 420 atop each other. For example, in a laser ablation process, machining the implant surface 400, at least first two adjacent ablation layers, patches 410, form a first joint or a first gap 420, and second two adjacent ablation layers, patches 410, form a second joint or a second gap 420, the first two adjacent ablation layers, patches 410, and the second two adjacent ablation layers, patched 410, are placed mainly atop of each other so that a cumulative build-up of the first joint or the first gap 420, and of the second joint or a second gap 420 is avoided.

Without limiting the foregoing and unless the context requires otherwise, all features or other elements as disclosed herein with respect to FIG. 4 shall be deemed herewith disclosed in any combination with each other, in any partial combination with each other, and in any order; and shall be deemed herewith disclosed in combination with all features or other elements as disclosed herein with respect to FIGS. 1 to 3, and 5 to 14 in any combination, in any partial combination, and in any order. Without limiting the foregoing and unless the context requires otherwise, the implant surface 400 referenced with respect to FIG. 4 may represent the same or similar surfaces, e.g., 110, 112, 114, 116, 120, 160, 161, 164, 166, 170, 210, 826, 868, or 924, or like elements as shown in and/or described with respect to FIGS. 1 to 3, 8, 9, and other figures herein, respectively. Without limiting the foregoing and unless the context requires otherwise, the medical device manufacturing system, the laser system, the laser beam, the focused laser beam, the laser ablation pool, the workpiece, the technical specification, the computer numerical control (CNC) data, the computer-executable instructions, the clinical imaging data, the patient, the custom-shape, the one or more non-transitory processor-readable medium, the non-transitory processor-readable memory, the one or more non-transitory computer data signals, the galvanometer, the mirror, the optical system, the intensity profile, the machine axis or axes, the stage, the focus shifter, the macroscale shape, the spatial extension, the mammal anatomy, the ablation layers, the patch 410, and the laser ablation layer, as each referenced with respect to FIG. 4 may represent the same or similar medical device manufacturing system, e.g., 500, 700, and 1080, the same or similar laser system, e.g., 500, and 770, the same or similar laser beam, e.g., 330, 500, 512, and 612, the same or similar focused laser beam, e.g., 330, 612, 820, 840, 860, 930, 950, and 979, the same or similar laser ablation pool, e.g., 600, and 866, the same or similar workpiece, e.g., 100, 150, 310, 520, 620, 780, 822, 922, and 1090, the same or similar technical specification, the same or similar computer numerical control (CNC) data, e.g., 1070, the same or similar computer-executable instructions, the same or similar clinical imaging data, e.g., 1024, and 1025, the same or similar patient, e.g., 1010, the same or similar custom-shape, e.g., 160, 162, and 164, the same or similar non-transitory processor-readable medium, e.g., 1024, 1026, 1056, 1066, 1024, 1030, 1032, 1040, 1059, 1069, and 1070, the same or similar non-transitory processor-readable memory, e.g., 1056, and 1066, the same or similar non-transitory computer data signal, e.g., 1024, 1025, 1032, 1059, 1069, and 1070, the same or similar galvanometer, the same or similar mirror, e.g., 330, and 332, the same or similar optical system, e.g., 534, the same or similar intensity profile, e.g., 514, the same or similar machine axis or axes, e.g., 720, 730, 740, 750, and 760, the same or similar stage, e.g., 720, 730, 740, 750, and 760, the same or similar focus shifter, the same or similar macroscale shape, e.g., 110, 112, 114, 116, 160, 162, and 164, the same or similar spatial extension, the same or similar mammalian anatomy, e.g., 1012, the same or similar ablation layers, the same or similar patch, the same or similar laser ablation layer, or the same and similar like elements, respectively, as shown in and/or described with respect to FIGS. 1 to 3, 5 to 10, 14, and other figures herein, respectively, and vice versa. Without limiting the foregoing and unless the context requires otherwise, the laser ablation process, the machining, the manufacturing process, and the various method steps referenced with respect to FIG. 4 may represent the same or similar laser ablation process, e.g., 300, 540, 600, 612, 820, 840, 860, 930, 950, and 979, the same or similar machining, the same or similar manufacturing process, e.g., 1080, and 1400, and the various same or similar method steps, e.g., 1410, 1420, 1430, 1440, 1450, or 1460, or the same or similar like elements, respectively, as described and/or shown with respect to FIGS. 1, 2, 10, 14, and other figures herein, respectively, and vice versa.

FIG. 5 shows an illustration of an exemplary embodiment of a laser system, in accordance with one or more embodiments of the presently disclosed technology, and, unless the context requires otherwise, combinable with any other embodiment disclosed herein.

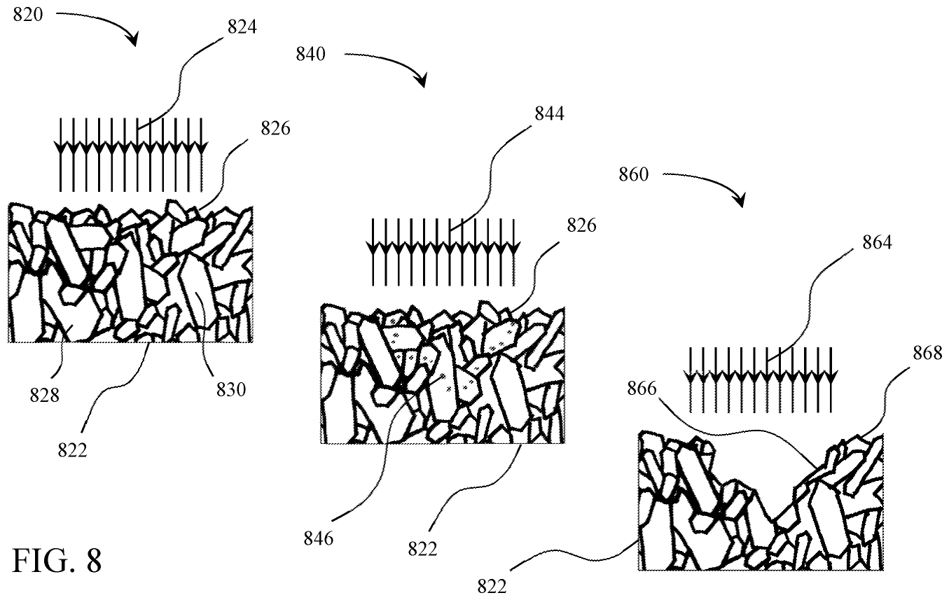

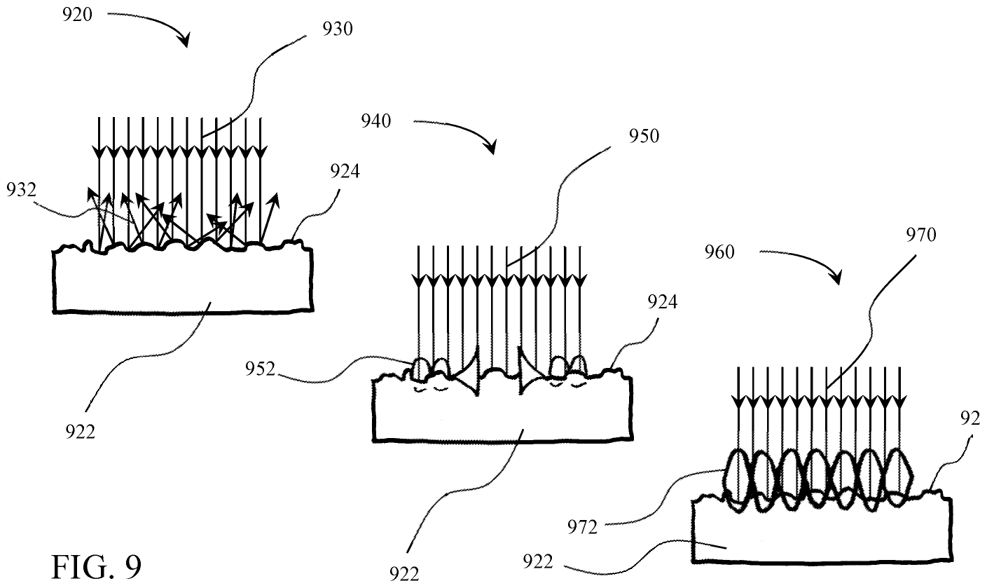

In one or more exemplary embodiments of the present technology, unless the context requires otherwise, combinable with any other embodiment disclosed herein, the laser system 500, may use a laser beam 510, that is, for example, widened after being originated by a laser source, for example from 3.9 mm to 14 mm. The laser beam may have a wavelength in the range between infrared and ultraviolet, for example, about 1030 nanometer. The laser beam 510 may be continuous or pulsed, where the pulse duration may range from 5 femtoseconds to 1 milliseconds, for example about 1 picoseconds, for example at a repetition rate of 1 MHz. A laser beam source might be an ultra-short-pulsed laser, for example a femto-second laser, for example, a water-cooled CARBIDE CB-3 (LIGHT CONVERSION, Vilnius, Lithuania), having an average output power of, for example, 80 W, a central wavelength, for example, 1030 nm, a maximum pulse energy of, for example, 400 μJ at 100 kHz. The laser beam 510 may be deflected by a mirror system 530, and 532 and focused onto a workpiece 520 to form a focused laser beam 512, having, for example, a Gaussian energy intensity or fluence distribution 514. The absorption of the laser photons of the focused laser beam 514 may excite the atoms of the material surface of the workpiece 520, generating subsequently, for example, very localized rapid heating and expansion. If the energy intensity or fluence is low, a high-pressure shockwave may cause the material to fracture and to eject a thin layer of material from the surface, also referred herein to as laser spallation, further described with reference to FIG. 8. At a higher fluence, the material may melt or may be directly vaporized and even ionized to form a plasma, and, by the inheriting local pressure of the process maybe ejected from the surface, which is also referred herein to as laser ablation, further described with reference to FIGS. 6, and 9. The optical lens system 534 may be an F-theta lens 534, for example, a telecentric F-theta lens 534, for example, a F-Theta JENar™ Silverline™ Lens 160-1030 . . . 1080-110 (e.g., JENOPTIK, Jena, Germany), having for example a focal length of 160 mm, and a focus size diameter of 22 micrometer. The optical lens system 534 may be an optical system operational to perform Bessel beam shaping, for example, including an axicon lens, or operational to create otherwise a Bessel beam 514 from a laser beam 510. The workpiece 520 may be an implant, for example, a dental implant as described in reference to FIG. 1, in a finished or semi-finished stage of manufacturing. A plurality of laser ablation spots 540 or laser spallation spots 540 may be positioned and arranged next to each other on the surface of the workpiece 520 by deflecting the laser beam 510, for example in rows and columns, using the mirrors 530, and 532. Each of the mirrors 530, and 532 may be rotated around an axis (as indicated in FIG. 5) by a servo-controlled galvanometer. The two rotational axes of the mirrors 530, and 532 may be oriented perpendicular in relation to each other. For example, a two-axes system in XY configuration may be used, for example, an excelliSCAN14 (e.g., SCANLAB, Puchheim, Germany), having in combination with a F-Theta lens with a focal length of 160 mm a marking speed of up to 4 m/s, and a scanning or image field on the surface of the workpiece 520 of up to 95 mm×95 mm. The mirrors 530, and 532 may be rotated responsive to computer numerically controlled (CNC) data, causing the laser beam 510 to be deflected and thereby position the ablation spot 540 induced by the focused laser beam 512 in a scan pattern, operable to ablate a layer of material of the workpiece 520. Such layer may have a thickness of about 2 micrometer. A control unit operable to drive the galvanometer scanner and the mirror system 530, and 532 in coordination with the triggering the laser source and thereby the laser beam 510 pulses may be operable to cause the laser system 500 to perform a laser ablation process layer-by-layer, for example, with an intermitted laser pulsing, to thereby engrave or form a macroscale shape, a microscale texture, and/or a nanoscale topography onto a surface of the workpiece 520. A Z-shifter, for example, an excelliSHIFT14 (e.g., SCANLAB, Puchheim, Germany) may be positioned in the path of the laser beam 510 may be operable to shift the focus range plus/minus 14 mm via the control unit responsive to computer numerically controlled (CNC) data, so that the laser system 500 may be operable to ablate non-even, surfaces. Taken together, the laser system 500, including the galvanometer mirrors 530 and 532 and the Z-shifter may by operable to perform for a 3-axis movement (x, y and z) of the focus of the focused laser beam 512, responsive to computer numerical control (CNC) instructions.

In one embodiment of the presently disclosed technology, unless the context requires otherwise, combinable with any other embodiment disclosed herein, the laser system 500 includes a pulsed femtosecond laser or a continuous beam laser. A laser source unit may include a gas laser, a solid-state laser, a fiber laser, a liquid laser, or a semiconductor laser. The laser system 500 may be used to form a macroscale shape, microscale surface texture, or nanoscale surface topography of the implant described in FIG. 1. It may also be used to form functionalized implant surfaces as described in FIG. 2. The laser source may function through an ablation process as described in FIG. 6. It may also create a spallation process as described in FIG. 8, or a laser interferometry process as described in FIG. 9. It may also be used to create a pore, or plurality of pores as described in FIG. 3. Additionally, it may be used to form a plurality of laser ablation patterns as described in FIG. 4. The focused laser 512 may shape and/or functionalize a surface of the workpiece 520 layer-by-layer in a plurality of layers each constituting a two-dimensional cross section of a solid object having an edge defined by a virtual three-dimensional model of the desired shape, texture and/or topography of the surface of the workpiece. Movement of the beam 510 across galvanometer mirrors 530 and 532 may also be controlled using feedback from a real-time, or near real-time sensor, such as, but not limited to, a sensor for surface topography, e.g., optical profilometer.

Without limiting the foregoing and unless the context requires otherwise, all features or other elements as disclosed herein with respect to FIG. 5 shall be deemed herewith disclosed in any combination with each other, in any partial combination with each other, and in any order; and shall be deemed herewith disclosed in combination with all features or other elements as disclosed herein with respect to FIGS. 1 to 4, and 6 to 14 in any combination, in any partial combination, and in any order. Without limiting the foregoing and unless the context requires otherwise, the workpiece 520 referenced with respect to FIG. 5 may represent the same or similar workpieces, e.g., 620, 710, 822 or like elements as shown in and/or described with respect to FIGS. 6 to 8 and other figures herein, respectively. Without limiting the foregoing and unless the context requires otherwise, the medical device manufacturing system, the laser system, the laser beam, the focused laser beam, the laser ablation pool, the workpiece, the technical specification, the computer numerical control (CNC) data, the computer-executable instructions, the clinical imaging data, the patient, the custom-shape, the non-transitory processor-readable medium, the one or more non-transitory processor-readable memory 1030, 1056, and 1066, the one or more non-transitory computer data signals 1024, 1032, 1059, 1069, and 1070, the galvanometer, the mirror, the optical system, the intensity profile, the machine axis or axes, the stage, the focus shifter, the macroscale shape, the spatial extension, the mammal anatomy, the material layers, the patch, and the laser ablation layer, as each referenced with respect to FIG. 5 may represent the same or similar medical device manufacturing system, e.g., 500, 700, and 1080, the same or similar laser system, e.g., 500, and 770, the same or similar laser beam, e.g., 330, and 612, the same or similar focused laser beam, e.g., 330, 612, 820, 840, 860, 930, 950, and 979, the same or similar laser ablation pool, e.g., 600, and 866, the same or similar workpiece, e.g., 100, 150, 310, 520, 620, 780, 822, 922, and 1090, the same or similar technical specification, the same or similar computer numerical control (CNC) data, e.g., 1070, the same or similar computer-executable instructions, the same or similar clinical imaging data, e.g., 1024, and 1025, the same or similar patient, e.g., 1010, the same or similar custom-shape, e.g., 160, 162, and 164, the same or similar non-transitory processor-readable medium, e.g., 1024, 1026, 1056, 1066, 1024, 1030, 1032, 1040, 1059, 1069, and 1070, the same or similar non-transitory processor-readable memory, e.g., 1056, and 1066, the same or similar non-transitory computer data signal, e.g., 1024, 1025, 1032, 1059, 1069, and 1070, the same or similar galvanometer, the same or similar mirror, e.g., 330, and 332, the same or similar optical system, e.g., 534, the same or similar intensity profile, e.g., 514, the same or similar machine axis or axes, e.g., 720, 730, 740, 750, and 760, the same or similar stage, e.g., 720, 730, 740, 750, and 760, the same or similar focus shifter, the same or similar macroscale shape, e.g., 110, 112, 114, 116, 160, 162, and 164, the same or similar spatial extension, the same or similar mammalian anatomy, e.g., 1012, the same or similar material layers, the same or similar patch, the same or similar laser ablation layer, or the same and similar like elements, respectively, as shown in and/or described with respect to FIGS. 1 to 4, 6 to 10, 14, and other figures herein, respectively, and vice versa. Without limiting the foregoing and unless the context requires otherwise, the laser ablation process, the machining, the manufacturing process, and the various method steps referenced with respect to FIG. 5 may represent the same or similar laser ablation process, e.g., 300, 600, 612, 820, 840, 860, 930, 950, and 979, the same or similar machining, the same or similar manufacturing process, e.g., 1080, and 1400, and the various same or similar method steps, e.g., 1410, 1420, 1430, 1440, 1450, or 1460, or the same or similar like elements, respectively, as described and/or shown with respect to FIGS. 1, 2, 10, 14, and other figures herein, respectively, and vice versa.

FIG. 6 shows an illustration of an exemplary embodiment of a laser ablation process, in accordance with one or more embodiments of the presently disclosed technology, and, unless the context requires otherwise, combinable with any other embodiment disclosed herein.

In one or more exemplary embodiments of the present technology, unless the context requires otherwise, combinable with any other embodiment disclosed herein, the laser ablation process 600 uses a focused laser beam 612, having a main axis of laser beam 610, to irradiate the surface 622 of the workpiece 620. A shaping of the surface 622 of the workpiece 620 using the laser ablation process 600 may be considered herein a machining of the workpiece 620 using a manufacturing process that includes subtractive shaping. The focused laser beam may at the surface a fluence at about or above the threshold required for laser ablation, causing the surface material 622 of the workpiece 620 to melt and/or to vaporize, and potentially to ionize, to thereby generate molten material 624 and/or material vapor and/or plasma 628. The pressure shockwave caused by the laser irradiation may eject molten material drops 630 and/or the ablation plume 632. The focused laser beam 612 may be continuous or pulsed. If pulsed, each pulse may leave a void or cavity 623 in the surface 622 of the workpiece 620. The void or cavity 623 resulting from a pulse of the focused laser beam 612 may have a diameter of 50 nanometer to 50 micrometer, for example, about 10 micrometer, and a depth of 10 nanometer to 20 micrometer, for example, about 2 micrometer. The laser irradiation may leave a heat affected zone 626 adjacent to the void or cavity 623. With an ultra-short pulsed focused laser beam 612, having a higher fluence, the ablated material may directly sublimate, and the heat affected zone 626 may be minimal. The laser ablation process 600 may be employed as part of a laser turning process. The laser ablation process 600 may be employed as part of a laser drilling process. The laser ablation process 600 may be employed as part of a laser percussion drilling process. A laser system 500, as described, for example, with respect to FIG. 5, may be operational to deflect the focused laser beam 612, for example, coordinated with the repetition pulse frequency of a laser source s pulse, pulse-by-pulse, for example, about 8 micrometer per pulse, to thereby form a plurality of void or cavities 623 adjacent each other, so that the cavities for a groove, or, an ablation layer, having, for example, an average depth between 1 micrometers and 2 micrometers. A laser system 500, as described, for example, with respect to FIG. 5, may be operational to use laser ablation process 600 to remove material or functionalize a surface 622 of the workpiece 620 layer-by-layer in a plurality of layers each constituting a two-dimensional cross section of a solid object having an edge defined by the digital data of the three-dimensional surface. A laser system 500, as described, for example, with respect to FIG. 5, may be operational to move the focused laser beam 612 using closed-loop control feedback from a real-time, or near real-time sensor, such as, but not limited to, a sensor for surface topography, e.g., optical profilometer.

Without limiting the foregoing and unless the context requires otherwise, an ablation process 600 may be used, for example, in combination with the laser system 500 from FIG. 5 and/or the 5-axis machine 700 from FIG. 7, to form a macroscale shape, a microscale surface texture and/or a nanoscale surface topography of an implant, for example, a dental implant 100, and 150, shown in and described with reference to FIG. 1. Without limiting the foregoing and unless the context requires otherwise, an ablation process 600 may also be used to form functionalized implant surfaces 210, 220, 225, and 255 as shown in and/or described with respect to FIG. 2. Without limiting the foregoing and unless the context requires otherwise, the process 600, depending, for example, on the fluence and the focal size of the focused laser beam, may also be used to perform a laser spallation process 820, 840, and 860 as shown in and/or described with respect to FIG. 8, or a laser interferometry process 920, 940, and 960 as shown in and/or described with respect to FIG. 9. Without limiting the foregoing and unless the context requires otherwise, an ablation process 600 may also be used to create a pore 322, or plurality of pores as shown in and/or described with respect to FIG. 3. Without limiting the foregoing and unless the context requires otherwise, an ablation process 600 may be used to form a plurality of laser ablation patterns 400, and 410 as shown in and/or described with respect to FIG. 4.

Without limiting the foregoing and unless the context requires otherwise, all features or other elements as disclosed herein with respect to FIG. 6 shall be deemed herewith disclosed in any combination with each other, in any partial combination with each other, and in any order; and shall be deemed herewith disclosed in combination with all features or other elements as disclosed herein with respect to FIGS. 1 to 5, and 7 to 14 in any combination, in any partial combination, and in any order. Without limiting the foregoing and unless the context requires otherwise, the workpiece 620 referenced with respect to FIG. 6 may represent the same or similar workpieces, e.g., 520, 710, 822 or like elements as shown in and/or described with respect to FIG. 5, 7, 8 and other figures herein, respectively. Without limiting the foregoing and unless the context requires otherwise, the implant body referenced with respect to FIG. 6 may represent the same or similar implant, e.g., 310, the same or similar workpiece, e.g., 100, 150, 520, 755, 822, 922, or the same and similar like elements, respectively, as shown in and/or described with respect to FIGS. 1, 3, 5, 7 to 9, 14, and other figures herein, respectively, and vice versa. Without limiting the foregoing and unless the context requires otherwise, the medical device manufacturing system, the laser system, the laser beam, the focused laser beam, the laser ablation pool, the workpiece, the technical specification, the computer numerical control (CNC) data 1070, the computer-executable instructions, the clinical imaging data, the patient, the custom-shape, the non-transitory processor-readable medium, the one or more non-transitory processor-readable memory 1030, 1056, and 1066, the non-transitory computer data signals 1024, 1032, 1059, 1069, and 1070, the galvanometer, the mirror, the optical system, the intensity profile, the machine axis or axes, the stage, the focus shifter, the macroscale shape, the spatial extension, the mammal anatomy, the ablation layers, the ablation 600, and the laser ablation layer, as each referenced with respect to FIG. 6 may represent the same or similar medical device manufacturing system, e.g., 500, 700, and 1080, the same or similar laser system, e.g., 500, and 770, the same or similar laser beam, e.g., 330, 500, and 512 the same or similar focused laser beam, e.g., 330, 820, 840, 860, 930, 950, and 979, the same or similar laser ablation pool, e.g. 866, the same or similar workpiece, e.g., 100, 150, 310, 520, 620, 780, 822, 922, and 1090, the same or similar technical specification, the same or similar computer numerical control (CNC) data, e.g., 1070, the same or similar computer-executable instructions, the same or similar clinical imaging data, e.g., 1024, and 1025, the same or similar patient, e.g., 1010, the same or similar custom-shape, e.g., 160, 162, and 164, the same or similar non-transitory processor-readable medium, e.g., 1024, 1026, 1056, 1066, 1024, 1030, 1032, 1040, 1059, 1069, and 1070, the same or similar non-transitory processor-readable memory, e.g., 1056, and 1066, the same or similar non-transitory computer data signal, e.g., 1024, 1025, 1032, 1059, 1069, and 1070, the same or similar galvanometer, the same or similar mirror, e.g., 330, and 332, the same or similar optical system, e.g., 534, the same or similar intensity profile, e.g., 514, the same or similar machine axis or axes, e.g., 720, 730, 740, 750, and 760, the same or similar stage, e.g., 720, 730, 740, 750, and 760, the same or similar focus shifter, the same or similar macroscale shape, e.g., 110, 112, 114, 116, 160, 162, and 164, the same or similar spatial extension, the same or similar mammalian anatomy, e.g., 1012, the same or similar ablation layers, the same or similar patch, the same or similar laser ablation layer, or the same and similar like elements, respectively, as shown in and/or described with respect to FIGS. 1 to 10, 14, and other figures herein, respectively, and vice versa. Without limiting the foregoing and unless the context requires otherwise, the laser ablation process 600 and 612 and the various method steps referenced with respect to FIG. 6 may represent the same or similar laser ablation process, e.g., 300, 540, 820, 840, 860, 930, 950, and 979, the same or similar machining, the same or similar manufacturing process, e.g., 1080, and 1400, and the various same or similar method steps, e.g., 1410, 1420, 1430, 1440, 1450, or 1460, or the same or similar like elements, respectively, as described and/or shown with respect to FIGS. 1, 2, 10, 14, and other figures herein, respectively, and vice versa.

FIG. 7 shows an illustration of an exemplary embodiment of a 5-axis machine, in accordance with one or more embodiments of the presently disclosed technology, and, unless the context requires otherwise, combinable with any other embodiment disclosed herein.

In one or more exemplary embodiments of the present technology, unless the context requires otherwise, combinable with any other embodiment disclosed herein, a machine 700 may include a machine frame 710, and directly or indirectly attached thereto, for example, a linear X-axis stage 720 with an X-axis travel path 725, a linear Y-axis stage 730 with a Y-axis travel path 735, a linear Z-axis stage 760 with a Z-axis travel path 765, a rotary A-axis swivel stage 740 with an A-axis travel path 745, and/or a rotary C-axis stage 750 with a C-axis travel path 755, or any combination thereof, each as shown in FIG. 7 or operational otherwise. The machine 700 may further include a machining spindle 770, or a laser system 770, for example, attached to the linear Z-axis stage 760. A workpiece 780 may be attached, directly or indirectly, by, for example, a collet holder, or a machine vice, to any of the stages 720, 730, 740, 750, and 760. All stages may be, for example, servo controlled by a control unit, operable to move the workpiece 780, in relation and in coordination with the machining spindle 770, or the laser system 770, responsive to computer numerical control data. A machine 700 may also be operational or otherwise configured to patch or map a plurality of laser ablation layers, responsive to computer numerical control data, onto a circumferential extension of the workpiece 780, thus machining a shape of the workpiece 780, for example, an implant, or a dental implant, so that a shape of the workpiece 780 corresponds to a virtual shape model of the implant, or the dental implant. The laser system 770 may remove material or functionalize a surface of the workpiece 780 layer-by-layer in a plurality of layers each constituting a two-dimensional cross section of a solid object having an edge defined by the digital data of the three-dimensional surface. Movement of the machine axis or axes, e.g., 720, 730, 740, 750, and 760, or the stage, e.g., 720, 730, 740, 750, and 760, may also be controlled using feedback from a real-time, or near real-time sensor, such as, but not limited to, a sensor for surface topography, e.g., optical profilometer.

Without limiting the foregoing, and in an exemplary embodiment, unless the context requires otherwise, combinable with any other embodiment disclosed herein, the machine 700 is used with 4 stages, e.g., 710, 720, 730, and 750, so that the machine 700 is operational to perform a laser turning process shaping the workpiece 780. The machine 700 may be used with 3 stages, e.g., 710, 720, and 730 to shape the workpiece 780 to form an implant, for example, a dental implant 100, and 150 as shown in and/or described with respect to FIG. 1. The machine 700 may be combined with the laser system described in FIG. 5 to further add 3 axes from the laser source, to be operational as an 8-axis machine 700, for example, a high-performance laser processing center RDX800 (e.g., Pulsar Photonics, Herzogenrath, Germany) having, for example, a Cartesian axis system XYZ, a combined rotary and swivel unit, a control cabinet with machine control, an operator terminal, 3D laser processing head and an F-Theta optics, and/or a DLIP laser processing head, a high power ultra-short-pulsed 80 W laser source, and machine control software, and a computer aided manufacturing (CAM) design, operational for micromachining of complex three-dimensional parts, such as implants.

Without limiting the foregoing and unless the context requires otherwise, all features or other elements as disclosed herein with respect to FIG. 7 shall be deemed herewith disclosed in any combination with each other, in any partial combination with each other, and in any order; and shall be deemed herewith disclosed in combination with all features or other elements as disclosed herein with respect to FIGS. 1, to 6, and 8 to 14 in any combination, in any partial combination, and in any order. Without limiting the foregoing and unless the context requires otherwise, the workpiece 780 referenced with respect to FIG. 7 may represent the same or similar workpiece, e.g., 310, 520, 620, 822, or 922, the same or similar implant, e.g., 100, or 150, or 1090, or the same and similar like elements, respectively, as shown in and/or described with respect to FIGS. 1, 3, 5, 6, 8 to 10, 14, and other figures herein, respectively, and vice versa. Without limiting the foregoing and unless the context requires otherwise, the workpiece surface referenced with respect to FIG. 7 may represent the same or similar surfaces, e.g., 110, 112, 114, 116, 120, 160, 161, 164, 166, 170, 210, 400, 826, 868, or 924, or like elements as shown in and/or described with respect to FIGS. 1 to 4, 8 to 10, and other figures herein, respectively. Without limiting the foregoing and unless the context requires otherwise, the manufacturing system, the computer numerical control (CNC) instructions, the machine stages, the galvanometer mirrors, the laser system, and the multi-axes machine system, as each referenced with respect to FIG. 7 may represent, the same or similar manufacturing system, e.g., 300, 500, and 1080, the same or similar computer numerical control (CNC) instructions, e.g., 1070, the same or similar machine stages, the same or similar galvanometer mirrors, e.g., 530, and 532, the same or similar laser system, e.g., 500, the same or similar multi-axes machine system, e.g., 500, respectively, or the same or similar like elements, respectively, as described and/or shown with respect to FIGS. 3, 5, 6, 8, 9, and other figures herein, respectively, and vice versa.

FIG. 8 shows an illustration of exemplary embodiments of a laser spallation process, in accordance with one or more embodiments of the presently disclosed technology, and, unless the context requires otherwise, combinable with any other embodiment disclosed herein.

In one or more exemplary embodiments of the present technology, unless the context requires otherwise, combinable with any other embodiment disclosed herein, a first laser spallation process scenario 820 comprises a workpiece 822 where a focused laser beam pulse 824 may irradiate the surface 826 of the workpiece 822 and does not (yet) affect the grain structure 828, and 830. A second laser spallation process scenario 840 is depicted including the same workpiece 822 where a subsequent laser beam pulse 844 or a series of subsequent laser beam pulses 844 induces microscale or nanoscale cracks 846 in the grain structure 828, and 830 adjacent the surface 826 of the workpiece 822 and may further a crack propagation 846 across the grain structure 828, and 830. A third laser spallation process scenario 860 is depicted including the same workpiece 822 where a further subsequent laser beam pulse 864 or a series of subsequent laser beam pulses 864 may further the crack propagation 846 so that a material portion is spalled off the surface 826 forming surface 868 including a laser spallation cavity 866. In this context and throughout this disclosure, the term “laser spallation” and derivative or similar words shall be understood as being generic to all possible meanings supported by the specification and by the words itself; the meaning shall, however, include herein, without limiting the foregoing and unless the context requires otherwise, a process of irradiating a body of material with a laser beam to thereby randomly spall particulates from the body of material. By way of example, and not limitation, an ultrashort pulsed laser can be used predominantly below the laser ablation threshold, utilizing a process of laser photon absorption to create predominate stress within the material causing the detachment of the spalled particulate.

Without limiting the foregoing, and in an exemplary embodiment, unless the context requires otherwise, combinable with any other embodiment disclosed herein, a laser spallation process 820, 840, and 860 may be used to form a pattern of a plurality of laser spallation cavities 866 onto a surface 826, and 868 of an implant 822, for example, microscale texture or a nanoscale topography of a dental implant. The pattern may be randomly distributed and/or correlate to a grain structure 828, and 830 of a material of the workpiece 822. In this context and throughout this disclosure, the term “stochastic laser material removal” and derivative or similar words shall be understood as being generic to all possible meanings supported by the specification and by the words itself; the meaning shall, however, include herein, without limiting the foregoing and unless the context requires otherwise, a process of irradiating a body of material with a laser beam to thereby randomly remove material from the body of material. Stochastic laser material removal processes may include a laser spallation process 820, 840, and 860. A laser spallation process 820, 840, and 860 may be used in combination with the laser system 500 from FIG. 5 and/or the 5-axis machine from FIG. 7 to form a macroscale shape, microscale surface texture, or nanoscale surface topography of the implant described in FIG. 1. A laser spallation process 820, 840, and 860 may be used to form functionalized implant surfaces as described in FIG. 2. A laser spallation process 820, 840, and 860 may be used to form a laser pattern 400, 410 as shown in and/or described with respect to FIG. 4.

Without limiting the foregoing and unless the context requires otherwise, all features or other elements as disclosed herein with respect to FIG. 8 shall be deemed herewith disclosed in any combination with each other, in any partial combination with each other, and in any order; and shall be deemed herewith disclosed in combination with all features or other elements as disclosed herein with respect to FIGS. 1 to 7, and 9 to 14 in any combination, in any partial combination, and in any order. Without limiting the foregoing and unless the context requires otherwise, the workpiece 822 referenced with respect to FIG. 8 may represent the same or similar workpieces, e.g., 520, 620, 710 or like elements as shown in and/or described with respect to FIGS. 5 to 7, and other figures herein, respectively. Without limiting the foregoing and unless the context requires otherwise, the implant body referenced with respect to FIG. 7 may represent the same or similar implant, e.g., 310, the same or similar workpiece, e.g., 100, 150, 520, 620, 755, 822, 922, or the same and similar like elements, respectively, as shown in and/or described with respect to FIGS. 1, 3, 5, 6, 7, 9, 14, and other figures herein, respectively, and vice versa. Without limiting the foregoing and unless the context requires otherwise, the medical device manufacturing system, the laser system, the laser beam, the focused laser beam, the laser ablation pool, the workpiece, the technical specification, the computer numerical control (CNC) data, the computer-executable instructions, the clinical imaging data, the patient, the custom-shape, the non-transitory processor-readable medium, the non-transitory processor-readable memory, the non-transitory computer data signal, the galvanometer, the mirror, the optical system, the intensity profile, the machine axis or axes, the stage, the focus shifter, the macroscale shape, the spatial extension, the mammal anatomy, the ablation layers, the ablation 820, 840, and 860, and the laser ablation layer, as each referenced with respect to FIG. 8 may represent the same or similar medical device manufacturing system, e.g., 500, 600, 700, and 1080, the same or similar laser system, e.g., 500, and 770, the same or similar laser beam, e.g., 330, 500, and 512 the same or similar focused laser beam, e.g., 330, 820, 840, 860, 930, 950, and 979, the same or similar laser ablation pool, e.g., 600 the same or similar workpiece, e.g., 100, 150, 310, 520, 620, 780, 822, 922, and 1090, the same or similar technical specification, the same or similar computer numerical control (CNC) data, e.g., 1070, the same or similar computer-executable instructions, the same or similar clinical imaging data, e.g., 1024, and 1025, the same or similar patient, e.g., 1010, the same or similar custom-shape, e.g., 160, 162, and 164, the same or similar non-transitory processor-readable medium, e.g., 1024, 1026, 1056, 1066, 1024, 1030, 1032, 1040, 1059, 1069, and 1070, the same or similar non-transitory processor-readable memory, e.g., 1056, and 1066, the same or similar non-transitory computer data signal, e.g., 1024, 1025, 1032, 1059, 1069, and 1070, the same or similar galvanometer, the same or similar mirror, e.g., 330, and 332, the same or similar optical system, e.g., 534, the same or similar intensity profile, e.g., 514, the same or similar machine axis or axes, e.g., 720, 730, 740, 750, and 760, the same or similar stage, e.g., 720, 730, 740, 750, and 760, the same or similar focus shifter, the same or similar macroscale shape, e.g., 110, 112, 114, 116, 160, 162, and 164, the same or similar spatial extension, the same or similar mammalian anatomy, e.g., 1012, the same or similar ablation layers, the same or similar patch, the same or similar laser ablation layer, or the same and similar like elements, respectively, as shown in and/or described with respect to FIGS. 1 to 10, 14, and other figures herein, respectively, and vice versa. Without limiting the foregoing and unless the context requires otherwise, the laser ablation process 820, 840, and 860, and the various method steps referenced with respect to FIG. 8 may represent the same or similar laser ablation process, e.g., 300, 540, 600, 612, 930, 950, and 979, the same or similar machining, the same or similar manufacturing process, e.g., 1080, and 1400, and the various same or similar method steps, e.g., 1410, 1420, 1430, 1440, 1450, or 1460, or the same or similar like elements, respectively, as described and/or shown with respect to FIGS. 1, 2, 10, 14, and other figures herein, respectively, and vice versa.

FIG. 9 shows an illustration of exemplary embodiments of a laser ablation process based on laser interferometry, in accordance with one or more embodiments of the presently disclosed technology.

In one or more exemplary embodiments of the present technology, unless the context requires otherwise, combinable with any other embodiment disclosed herein, a first interferometric laser process scenario 920 comprises a workpiece 922 where a laser beam 930 irradiates a surface 924 of a workpiece 922. The surface 924 may be uneven or have a surface roughness 924 causing the laser beam 930 to reflect from the surface as a scatter 932. A second interferometric laser process scenario 940 is depicted including the same workpiece 922 where the laser beam 960 continues initiating a resonance 952 adjacent the surface 924 of the workpiece 922. A third interferometric laser process scenario 960 is depicted including the same workpiece 922 where the laser beam 970 continues to form a laser interference pattern, amplifying partially the fluence above the threshold that may cause a partial ablation process 960 on the surface 924 of the workpiece 922 forming, for example, and without limiting the foregoing, laser-induced periodic surface structures (LIPSS), for example, a nanoscale topography 250, and 255 as shown in and/or described with respect to FIG. 2.

Without limiting the foregoing, and in an exemplary embodiment, unless the context requires otherwise, combinable with any other embodiment disclosed herein, a laser ablation interferometry process 920, 940, 960 may be used in combination with the laser system 500 as shown in and/or described with respect to FIG. 5 and/or the 5-axis machine 700 as shown in and/or described with respect to FIG. 7 to form a macroscale shape, a microscale surface texture, or a nanoscale surface topography of an implant, for example, a dental implant 100, and 150, as shown in and/or described with respect to in FIG. 1. A laser ablation interferometry process 920, 940, 960 may be used to form a functionalized implant surface 225 as shown in and/or described with respect to FIG. 2. A laser ablation interferometry process 920, 940, 960 may be used to form a laser ablation pattern 400, 410 as shown in and/or described with respect to FIG. 4.

Without limiting the foregoing and unless the context requires otherwise, all features or other elements as disclosed herein with respect to FIG. 9 shall be deemed herewith disclosed in any combination with each other, in any partial combination with each other, and in any order; and shall be deemed herewith disclosed in combination with all features or other elements as disclosed herein with respect to FIGS. 1, to 8, and 10 to 14 in any combination, in any partial combination, and in any order. Without limiting the foregoing and unless the context requires otherwise, the workpiece 922 referenced with respect to FIG. 9 may represent the same or similar workpieces, e.g., 520, 620, 710, 822, or like elements as shown in and/or described with respect to FIGS. 5 to 8, and other figures herein, respectively. Without limiting the foregoing and unless the context requires otherwise, the implant body referenced with respect to FIG. 9 may represent the same or similar implant, e.g., 310, the same or similar workpiece, e.g., 100, 150, 520, 620, 755, 822, or the same and similar like elements, respectively, as shown in and/or described with respect to FIGS. 1, 3, 5, 6, 8, 14, and other figures herein, respectively, and vice versa. Without limiting the foregoing and unless the context requires otherwise, the medical device manufacturing system, the laser system, the laser beam, the focused laser beam, the laser ablation pool, the workpiece, the technical specification, the computer numerical control (CNC) data, the computer-executable instructions, the clinical imaging data, the patient, the custom-shape, the non-transitory processor-readable medium, the non-transitory processor-readable memory, the non-transitory computer data signal, the galvanometer, the mirror, the optical system, the intensity profile, the machine axis or axes, the stage, the focus shifter, the macroscale shape, the spatial extension, the mammal anatomy, the ablation layers, the interferometry ablation 930, 950, and 979, and the laser ablation layer, as each referenced with respect to FIG. 9 may represent the same or similar medical device manufacturing system, e.g., 500, 600, 700, and 1080, the same or similar laser system, e.g., 500, and 770, the same or similar laser beam, e.g., 330, 500, and 512 the same or similar focused laser beam, e.g., 330, 820, 840, 860 the same or similar laser ablation pool, e.g. 866, the same or similar workpiece, e.g., 100, 150, 310, 520, 620, 780, 822, 922, and 1090, the same or similar technical specification, the same or similar computer numerical control (CNC) data, e.g., 1070, the same or similar computer-executable instructions, the same or similar clinical imaging data, e.g., 1024, and 1025, the same or similar patient, e.g., 1010, the same or similar custom-shape, e.g., 160, 162, and 164, the same or similar non-transitory processor-readable medium, e.g., 1024, 1026, 1056, 1066, 1024, 1030, 1032, 1040, 1059, 1069, and 1070, the same or similar non-transitory processor-readable memory, e.g., 1056, and 1066, the same or similar non-transitory computer data signal, e.g., 1024, 1025, 1032, 1059, 1069, and 1070, the same or similar galvanometer, the same or similar mirror, e.g., 330, and 332, the same or similar optical system, e.g., 534, the same or similar intensity profile, e.g., 514, the same or similar machine axis or axes, e.g., 720, 730, 740, 750, and 760, the same or similar stage, e.g., 720, 730, 740, 750, and 760, the same or similar focus shifter, the same or similar macroscale shape, e.g., 110, 112, 114, 116, 160, 162, and 164, the same or similar spatial extension, the same or similar mammalian anatomy, e.g., 1012, the same or similar ablation layers, the same or similar patch, the same or similar laser ablation layer, or the same and similar like elements, respectively, as shown in and/or described with respect to FIGS. 1 to 8, 10, 14, and other figures herein, respectively, and vice versa. Without limiting the foregoing and unless the context requires otherwise, the laser ablation process 820, 840, and 860, and the various method steps referenced with respect to FIG. 8 may represent the same or similar laser ablation process, e.g., 300, 540, 600, 612, 820, 840, and 860, the same or similar machining, the same or similar manufacturing process, e.g., 1080, and 1400, and the various same or similar method steps, e.g., 1410, 1420, 1430, 1440, 1450, or 1460, or the same or similar like elements, respectively, as described and/or shown with respect to FIGS. 1, 2, 10, 14, and other figures herein, respectively, and vice versa.

FIG. 10 shows a schematic block diagram illustrating an exemplary embodiment of an implant treatment and related information technology, (IT), computer architecture, network environment, and other systems configuration and method steps in accordance with one or more embodiments of the presently disclosed technology.

The system 1000 presented in FIG. 10 indicates in one or more exemplary embodiments of the presently disclosed technology, unless the context requires otherwise, combinable with any other embodiment disclosed herein, a workflow, method steps, the flow of data, the acquisition of image data, the generation of a treatment plan, the CAD/CAM design and manufacturing and delivery of a dental implant and the supporting information technology (IT) configuration assisting in the implant treatment, for example, for a rehabilitation of a dentition 1012 of a patient 1010. In this context and throughout this disclosure, the term “implant” and derivative or similar words shall be understood herein as being generic to all possible meanings supported by the specification and by the words itself; the meaning shall, however, include, without limiting the foregoing and unless the context requires otherwise, artificial, or artificially enhanced objects which are temporarily, or permanently placed in mammalian tissue by a medical procedure or surgery. For example, utilizing a handpiece of a 3D intraoral scanner 1020 connected to its control unit 1022, a spatial image of anatomical surfaces of the upper and/or lower jaw and its respective teeth (e.g., including a bite registration) of the dentition 1012 may be taken, the 3D scan data 1024, may be transferred 1032 through the internet 1040 or a local network to a (for example, cloud-based) data storage system including a database 1030. Alternatively or additionally, for example, anatomical structures of and adjacent to the dentition 1012 of the patient 1010 may be X-rayed using a cone beam computer tomography (CBCT) machine 1021 connected to its control unit 1023, and the 3D image data 1025, may be transferred 1032 through the internet 1040 or a local network to a (for example, cloud-based) data storage system, including a database 1030. Further, for example, anatomical structures of the patient 1010 may be imaged by a clinical imaging unit connected to its respective control unit, and 2D, 3D, and/or 4D image data, may be captured and transferred through the internet 1040 or a local network to a (for example, cloud-based) data storage system, including a database 1030. Suitable clinical image units may use computer tomography (CT), cone-beam CT (CBCT) 1021, magnetic resonance imaging (MRI), ultrasound, active triangulation, passive triangulation, confocal scanning, and time-of-flight (TOF) technologies. In this context and throughout this disclosure, the term “image data” and derivative or similar words shall be understood as being generic to all possible meanings supported by the specification and by the words itself; the meaning shall, however, include herein, without limiting the foregoing and unless the context requires otherwise, any numerical or computer-implemented two-dimensional, three-dimensional, or four-dimensional description, representation, reproduction, or imitation of the appearance of a mammalian anatomy, or in general, a thing, or respective parts thereof. In this context and throughout this disclosure, the term “3D”, “three-dimensional” and derivative or similar words shall be understood as being generic to all possible meanings supported by the specification and by the words itself; the meaning shall, however, include herein, without limiting the foregoing and unless the context requires otherwise, something of two-dimensional extension and the changes thereof throughout time, or as something of three-dimensional, spatial extension, as the context suggests. The term “4D” or “four-dimensional” and derivative or similar words shall be understood herein as something of three-dimensional, spatial extension and the changes thereof throughout time. In this context and throughout this disclosure, the term “spatial” and derivative or similar words shall be understood as being generic to all possible meanings supported by the specification and by the words itself; the meaning shall, however, include herein, without limiting the foregoing and unless the context requires otherwise, one or more of: three-dimensional, relating to or occupying space, non-linear, non-planar, and/or being of natural physical real and non-mathematical shape. Image data may include data, including virtual surface data, and/or data representative of a surface. In this context and throughout this disclosure, the term “surface” and derivative or similar words shall be understood as being generic to all possible meanings supported by the specification and by the words itself; the meaning shall, however, include herein, without limiting the foregoing and unless the context requires otherwise, one or more of: an outside of a physical body, a virtual surface, a numerical representation of a surface, a virtual outside of a numerical representation of a body, a surface boundary, a transitional surface boundary, a virtual shape contour, a physical shape contour, a virtual edge contour, and/or a physical edge contour. As it relates to the terms “a virtual surface” and/or “a numerical representation of a surface” and derivative or similar terms, it should be understood that those surfaces have no inside or outside per se, to the extent that an inverse surface to such surface would be identical to such surface itself. Surface data may be represented as a plurality of the three-dimensional coordinates, a point cloud, a plurality of coordinated triangles, a mesh, or otherwise. Two-dimensional X-ray or MRI data may include a plurality of pixel, each pixel of that plurality may include a tissue density, or a greyscale value. Three-dimensional X-ray or MRI data may include a plurality of voxel, each voxel may include a include a tissue density, or a greyscale value. The scan and/or other image data may be subsequently made available 1059 to a personal computer (PC) or workstation for treatment planning 1050, comprising, for example, a computer monitor displaying, for example, various user interfaces, including the user interface for implant treatment planning 1052. A treatment plan 1059 that may comprise a prescription of an implant by the treating medical doctor, dentist or dental specialist and a model of the implant, and simulation of the implant site situation of the human anatomy, e.g., the dentition 1012 of the patient 1010. The treatment plan 1059 may be transferred through the internet 1040 or a local network and may be made available 1069 to personal computer (PC) or workstation, for CAD/CAM operations 1060, where the design of the dental implant may take place, and subsequently computer numerical control (CNC) data 1070, may be derived and transferred to a manufacturing facility 1080, where, for example, equipment for primary, additive, subtractive and/or forming manufacturing 1080 may be utilized to fabricate a prescribed implant 1090, for example, a dental implant 1090 according to one or more exemplary embodiments of the present technology disclosed herein, unless the context requires otherwise, combinable with any other embodiment disclosed herein, responsive to the design data 1069, and/or computer numerical control (CNC) data 1070. The implant 1090, e.g., the dental implant 1090 can then, for example, be delivered to the office of the medical doctor, for example, the treating dentist or dental specialist and may be inserted and integrated into the body, for example the dentition 1012 of a pre-identified patient 1010 for rehabilitation or augmentation, for example of rehabilitation of such patient's dentition 1012.

Each personal computer (PC) or workstation 1050, and 1060 may comprise a respective hardware and software configuration 1055, and 1065, comprising, for example, a memory 1056, and 1066, one or more processors 1057, and 1067, and a communications interface 1058, and 1068. The database 1030, for example a relational SQL database 1030, may be stored or be storable on the memory 1030, 1056, and 1066 may comprise one or more non-transitory processor-readable media, as described in detail above. The computer data 1024, 1032, 1059, 1069, and 1070, and the computer data transferred within the computer hardware configurations 1055, and 1065 and within the manufacturing facility 1080 may be transferred and/or transmitted by any one or more physical, transferrable, and reproducible computer data signal 1024, 1025, 1032, 1040, 1059, 1069, and 1070 as described in detail above utilizing any wired and wireless transmission device and technology, as described in detail above. The software configurations 1055, and 1065, may comprise one or more computer program products, including for example processor-readable instructions, that may be received by the processor(s) 1057, and 1067, for example, from one or more non-transitory processor-readable memory 1030, 1056, 1066, by means of one or more non-transitory processor-readable media, or by one or more non-transitory computer data signal. The computer program products may be articles of manufacture, and may comprise firmware, operating systems, and/or applications. The computer program product may encode a computer program for executing on a computer system and/or one or more processors 1057, and 1067 a computer process, comprising, for example, a plurality of functions. When implemented in software or firmware, various elements of the systems described herein can be code segments or instructions that perform the various tasks. The program or code segments can be stored or can be storable in a processor-readable medium or transmitted by a computer data signal embodied in a carrier wave over a transmission medium or communication path.

In one or more exemplary embodiments, unless the context requires otherwise, combinable with any other embodiment disclosed herein, a computer program product 1055, and 1065 is operational or otherwise configured to assist in an implant treatment of a pre-identified patient 1010, the computer program product 1055, and 1065 encoding a computer program for executing on a computer system 1050, and 1060 a computer process, the computer process comprising: receiving first numerical data 1024, 1032, 1059, and 1069 comprising clinical data 1024, 1025, and an initial technical specification of the implant. The clinical data 1024, 1025 may be representative of an anatomical structure 1012 of the pre-identified patient 1010. Automatic or interactive computer processes may process the first numerical data 1024, 1032, 1059, and 1069 for visualization, may enhance and may combine clinical data 1024, 1025. Further, automatic or interactive computer processes may segment and label anatomical data, creating anatomical models responsive to the first numerical data 1024, 1032, 1059, and 1069, being, for example, specific to the anatomical structures of the pre-identifies patient 1010. Computer aided self-learning algorithms and/or artificial intelligence functions of computer processes may assist in the segmentation and labeling of the specific anatomical structures based on statistical models of anatomical structures. In this context and throughout this disclosure, the term “model” without any specifier shall be understood herein as a “virtual model” unless the context requires otherwise. The term “virtual model” and derivative or similar words shall be understood as being generic to all possible meanings supported by the specification and by the words itself; the meaning shall, however, include herein, without limiting the foregoing and unless the context requires otherwise, any numerical or computer-implemented description, representation, reproduction, or imitation of the past, present, or future state of something, for example in the given context of anatomical structures, or implant designs or portions thereof. The models may be required to have a substantial spatial extension. In this context and throughout this disclosure, the term “substantial spatial extension” and derivative or similar terms shall be understood as being generic to all possible meanings supported by the specification and by the words itself; the meaning shall, however, include herein, without limiting the foregoing and unless the context requires otherwise, any spatial extension that is not nonmaterial. Models, manufactured implants, accessory parts or portions thereof may correlate to spatial surfaces of anatomical structures of the pre-identifies patient 1010. In this context and throughout this disclosure, the term “correlating to a surface or shape” or “correlating to a corresponding surface” and derivative or similar terms shall be understood as being generic to all possible meanings supported by the specification and by the words itself; the meaning shall, however, include herein, without limiting the foregoing and unless the context requires otherwise, one or more of: substantially matching a similar shape of a corresponding surface, substantially matching a similar shape of a dimensionally reduced virtual representation of a corresponding surface, substantially matching a similar shape of a dimensionally expanded virtual representation of a corresponding surface, substantially matching a similar shape of an undersized virtual representation of a corresponding surface, and/or substantially matching a similar shape of an oversized virtual representation of a corresponding surface.

The computer system may comprise one or more processors 1057, and 1067. The processor(s) 1057, and 1067 may be operationally connected to a non-transitory processor-readable medium 1056, 1066, 1058, and 1068, 1059, and 1069 through or from which the processor(s) receives encoded computer program instructions, comprised in the computer program product. The non-transitory processor-readable medium 1056, 1066, 1058, and 1068, 1059, and 1069 may be a memory 1056, and 1066, for example a solid-state-drive (SSD) 1056, and 1066. The non-transitory processor-readable medium 1056, 1066, 1058, and 1068, 1059, and 1069 may be a computer data signal 1059, and 1069, transmitted, for example via a wireless local area network (WLAN) 1059, and 1069. The WLAN 1059, and 1069 may use a physical electromagnetic carrier signal to transfer the encoded program instructions, using a protocol that ensures the program instruction are reproducibly transferred.

In one or more exemplary embodiments of the presently disclosed technology, unless the context requires otherwise, combinable with any other embodiment disclosed herein, an implant 1090 is operational or otherwise configured for dental implantation. A numerical description of a first macroscale shape of the dental implant 1090 may be at least partially representative of an anatomical shape correlating to a crown of a pre-identified patient 1010, a transmucosal portion of a tooth of the pre-identified patient 1010, a gingival margin of the pre-identified patient 1010, or a bone crest adjacent an alveolar socket of the pre-identified patient 1010. A manufacturing process 1080 machining the first macroscale shape of the dental implant 1090 can use a process of customization so that the first macroscale shape of the dental implant 1090 correlates at least partially to the anatomical shape. During the design process the previously received technical specification that may not be patient-specific, may be updated to include at least a partial model of first macroscale shape of the dental implant. A method of manufacturing 1080 a customized dental implant for a pre-identified patient may be employed, the method comprising: obtaining a proposed specification of the dental implant 1090, the dental implant 1090 includes an endosseous root portion and an occlusal facing portion operational or otherwise configured to receive a dental prosthesis; obtaining a trained shape model, the trained shape model is descriptive of a statistical dental anatomy model, the statistical dental anatomy model includes one or more statistical dental anatomy element shape models; obtaining a data set including one or more virtual representations of one or more dental anatomy elements of a dentition of the patient; adapting at least a portion of the trained shape model to best-fit the one or more virtual representations to thereby form an adapted shape model; and updating the proposed specification 1059 of the dental implant 1090 responsive to the adapted shape model to thereby form an updated specification 1059.

Computer numerical control (CNC) data 1070 may be derived from the patient-specific design data or custom-shaped virtual models 1069 of the implant 1090, for example the dental implant 1090. The implant 1090, for example, the dental implant 1090 may be machined responsive to the computer numerical control (CNC) data 1070. In this context and throughout this disclosure, the term “machined”, “machining”, and derivative or similar words shall be understood as being generic to all possible meanings supported by the specification and by the words itself; the meaning shall, however, include herein, without limiting the foregoing and unless the context requires otherwise, a process of shaping a body of material by forming, removing or adding portions of material within, to or from the body of material. By way of example, and not limitation, an ultrashort pulsed laser is used to irradiate material with a focused laser beam to thereby, as a result of the laser/material interaction, melt and remove and/or sublimate the matter. Selective laser melting, selective laser sintering, stereo-lithography, CNC grinding, CNC turning, CNC laser or water cutting or shaping, CNC milling technologies, additive shaping technologies, subtractive shaping technologies, shape forming manufacturing technologies, primary shaping technologies, rapid prototyping and/or other machining and finishing technologies may also be considered non-limiting examples. In this context and throughout this disclosure, the term “rapid prototyping” and derivative or similar words shall be understood as being generic to all possible meanings supported by the specification and by the words itself; the meaning shall, however, include herein, without limiting the foregoing and unless the context requires otherwise, manufacturing technologies based on digital data, by a process that includes depositing material, in accordance with the digital data, layer-by-layer in a plurality of layers each constituting a two-dimensional cross section of a solid object having an edge defined by the digital data of the three-dimensional surface. By way of example, and not limitation, the layers from the two-dimensional surface may be stacked in a third dimension to form the solid object having a three-dimensional surface defined by the data. Rapid prototyping processes may be used for fabricating objects from more than one material. All such rapid prototyping technologies may be used directly to manufacture the part of interest, for example, by selective laser sintering. They may conversely be used indirectly by fabricating first, for example, a resin or wax sample of the part of interest that can be used, for example, to make the actual part by lost wax casing technology. The aforementioned processes may include sintering processes where a “green” body is 3D printed in response to computerized numerical controlled (CNC) data 1070 and then sintered to its final material properties. Sintering in this context may include pressure and heat. Further, the meaning of “rapid prototyping” shall include in its broadest technical sense, where individualized parts are made from virtual representations, and shall include respective primary, additive, subtractive and other forming technologies used to three-dimensionally shape workpieces. The meaning of “shape forming” shall include but shall not be limited to net shape or near net shape forming technologies, CNC stamping, CNC pressing, and CNC casting technologies. Each of the process steps 1420, 1440, and/or 1460 machining a workpiece to become an implant may be preceded by a process step of sintering a workpiece designated to become an implant. Manufacturing equipment may be based on multi-axis (e.g., 5-axis) operations. The implant surface of the implant 1090 may be functionalized according to one or more embodiments disclosed in this specification, unless the context requires otherwise, combinable with any other embodiment disclosed herein.

In the context of FIG. 10 and throughout this disclosure, embodiments of the subject matter may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. In practice, one or more processing systems or devices can carry out the described operations, tasks, and functions by manipulating electrical, and other processing, signals representing data bits at accessible memory locations. The memory locations where data bits are maintained are physical locations that have particular electrical, magnetic, optical, or organic properties corresponding to the data bits. It should be appreciated that the various block components of a control unit, a personal computer (PC) or a workstation 1050, 1060, a local or wide-area network 1040, a database 1030, and/or data storage system 1030 shown in FIG. 10 and in other figures herein may be realized by any number of hardware, software, and/or firmware components operational or otherwise configured to perform the specified functions. For example, an embodiment of a system or a component, unless the context requires otherwise, combinable with any other embodiment disclosed herein, may employ various integrated circuit components such as: memory elements, digital signal processing elements, logic elements, look-up tables, or the like. These components may carry out a variety of functions under the control of one or more microprocessors or other control devices. When implemented in software or firmware, various elements of the systems described herein can code segments or instructions that perform various tasks. The program or code segments can be stored or can be storable in a processor-readable medium and/or can be transmitted by a computer data signal embodied in a carrier wave over a transmission medium (or communication path). In this regard, the subject matter described herein can be implemented in the context of any computer-implemented system and/or in connection with two or more separate and distinct computer-implemented systems that cooperate and communicate with one another.

In one or more exemplary embodiments of the presently disclosed technology, unless the context requires otherwise, combinable with any other embodiment disclosed herein, an implant 1090 comprises a first functionalized surface operational or otherwise configured for integration into or with a first type of mammalian tissue. The first functionalized surface may comprise a first macroscale shape of spatial extension. The first functionalized surface may comprise a first machined microscale surface texture formed onto the first macroscale shape. The first functionalized surface may comprise a first nanoscale surface topography formed onto the first machined microscale surface texture. The implant 1090 may comprise an endosseous implant portion, the endosseous implant portion may include the first functionalized surface, and the first type of mammalian tissue predominately may include mammalian bone. The implant 1090 may comprise a transmucosal implant portion, the transmucosal implant portion may include the first functionalized surface, and the first type of mammalian tissue may predominately include mammalian mucosa. The implant 1090 may comprise a trans-cutaneous implant portion, the trans-cutaneous implant portion may include the first functionalized surface, and the first type of mammalian tissue predominately may include mammalian cutaneous or subcutaneous mammalian tissue. The implant 1090 may comprise a root portion, the root portion may include the first functionalized surface, and the first type of mammalian tissue may predominately include mammalian periodontal tissue. The implant 1090 may further comprise a second functionalized surface operational or otherwise configured for integration into or with a second type of mammalian tissue. The second functionalized surface may comprise a second macroscale shape of spatial extension. The second functionalized surface may comprise a second machined microscale surface texture formed onto the second macroscale shape. The second functionalized surface may comprise a second nanoscale surface topography formed onto the second machined microscale surface texture. The second type of mammalian tissue may be a different type than the first type of mammalian tissue. The implant 1090 may comprise an endosseous implant portion. The implant 1090 may comprise a transmucosal implant portion. The implant 1090 may be operational or otherwise configured for dental implantation. The endosseous implant portion includes the first functionalized surface. The transmucosal implant portion includes the second functionalized surface. The first type of mammalian tissue predominately includes human jawbone. The second type of mammalian tissue predominately includes human oral mucosa. The dental implant 1090 may be operational or otherwise configured as one-piece including an integral root portion, and an integral abutment portion. The integral root portion may include the endosseous implant portion. The integral abutment portion may include the transmucosal implant portion. The dental implant 1090 may be operational or otherwise configured as a two-piece assembly. The two-piece assembly may include a root portion, and an abutment that is detachable from the root portion. The root portion can include the endosseous implant portion. The abutment can include the transmucosal implant portion. An implant 1090 may comprise a root portion, and a transmucosal implant portion, and the implant 1090 may be operational or otherwise configured for dental implantation. The root portion may include the first functionalized surface. The transmucosal implant portion may include the second functionalized surface. The first type of mammalian tissue may predominately include human periodontal tissue. The second type of mammalian tissue may predominately include human oral mucosa. The dental implant 1090 may be operational or otherwise configured as one-piece including: the root portion, and an abutment portion integral with the root portion. The abutment portion may include the transmucosal implant portion. The dental implant 1090 may be operational or otherwise configured as a two-piece assembly, the two-piece assembly may include the root portion, and an abutment that is detachable from the root portion. The abutment can include the transmucosal implant portion. The first functionalized surface may be custom-shaped and can correlate to a shape of a root of a tooth of a pre-identified patient 1010, or to a shape of an alveolar socket 1012 of the pre-identified patient 1010. The second functionalized surface may be custom-shaped and can correlate to a shape of a tooth 1012 of a pre-identified patient 1010, to a shape of a gingival margin 1012 of the pre-identified patient 1010, or to a shape of a bone crest 1012 adjacent an alveolar socket of the pre-identified patient 1010. The first functionalized surface may include a deterministic laser ablation pattern. The first functionalized surface may include a periodic laser interferometric ablation pattern. The first functionalized surface may include a stochastic laser spallation pattern. The implant 1090 may include a body of material adjacent the first functionalized surface, the body of material may have a nanoscale crystallite grain structure. A structure of the first nanoscale surface topography may correlate at least partially to a structure of the nanoscale crystallite grain structure. The implant 1090 may predominantly include silicon nitride. The implant 1090 may predominantly include zirconia or alumina. The implant 1090 may include a substrate material. The first nanoscale surface topography may be at least partially erected by grains, crystals, crystallites, polymorphic aggregates, nano-pores, and/or amorphic aggregates included in the substrate material. The implant 1090 may include a thin film coating. The first nanoscale surface topography may be at least partially formed by grains, crystals, crystallites, polymorphic aggregates, nano-pores, and/or amorphic aggregates included in the thin film coating. The thin film coating may predominately include carbon.

In one or more exemplary embodiments of the presently disclosed technology, unless the context requires otherwise, combinable with any other embodiment disclosed herein, an implant system 1090 comprises a functionalized surface operational or otherwise configured for integration into or with mammalian tissue. The functionalized surface may comprise a machined macroscale shape of spatial extension. The functionalized surface may comprise a machined microscale surface texture formed onto the machined macroscale shape. The functionalized surface may comprise a nanoscale surface topography formed onto the machined microscale surface texture.

In one or more exemplary embodiments of the presently disclosed technology, unless the context requires otherwise, combinable with any other embodiment disclosed herein, an implant system 1090 comprises a functionalized surface operational or otherwise configured for integration into or with mammalian tissue. The functionalized surface may comprise a macroscale shape of spatial extension. The functionalized surface may comprise a machined microscale surface texture formed onto the macroscale shape. The functionalized surface may comprise a machined nanoscale surface topography formed onto the machined microscale surface texture.

In one or more exemplary embodiments of the presently disclosed technology, unless the context requires otherwise, combinable with any other embodiment disclosed herein, an implant 1090 comprises a functionalized surface operational or otherwise configured for integration into or with mammalian tissue. The functionalized surface may comprise a macroscale shape of spatial extension. The functionalized surface may comprise a microscale surface texture formed onto the macroscale shape. The functionalized surface may comprise a machined nanoscale surface topography formed onto the microscale surface texture.

In one or more exemplary embodiments of the presently disclosed technology, unless the context requires otherwise, combinable with any other embodiment disclosed herein, an implant 1090 comprises a functionalized surface operational or otherwise configured to reduce bacterial colonization. The functionalized surface may comprise a macroscale shape of spatial extension. The functionalized surface may comprise a machined microscale surface texture formed onto the macroscale shape. An implant 1090 may have at least partially a nanoscale topography formed on a microscale surface texture that is formed onto a macroscale shape, or formed directly onto a macroscale shape. Specific nanoscale topography surface structures, for example, laser-induced periodic surface structures (LIPSS) may create mechanical stress on bacteria affecting their morphology, and subsequently hindering their ability to spread and form biofilms. LIPSS may also enhance the proliferation of cells, promote alkaline phosphate formation, cell clustering, and/or filopodia attachment, promoting thereby tissue integration, for example, osseointegration. The nanoscale topography surface structures of functionalized surfaces of implants 1090 that reduce bacterial colonization may have an average surface roughness, Ra between 0.1 micrometer and 4 micrometer, for example about 0.2 micrometer. The average surface roughness may be machined. LIPSS may be machined by a process of direct laser interference patterning (DLIP) 1080, and may have a spatial extension as shown and/or discussed, for example, with respect to the periodic interference pattern 255 as depicted in FIG. 2. Further, functionalization of a surface of an implant 1090, as discussed herein in various embodiments, may affect and/or change the chemistry of a surface layer of a substrate material adjacent the functionalized surface of the implant 1090. For example, laser processes as discussed herein in various embodiments, may provide a depletion zone of the chemical composition and/or the availability of chemical substituents on the functionalized surface and/or adjacent the functionalized surface when compared to the substrate material of the implant 1090. Laser processes employed on sintered silicon nitride, Si₃N₄ as a substrate material of an implant 1090 may encourage zwitterionic-like material composition further encouraging cell growth. Functionalization of silicon nitride, Si₃N₄ as a substrate material of an implant 1090 may prime the surface for conversion of adjacent fluids to a bactericide, for example orthosilicic acid. Laser ablation and/or laser spallation processes may also reveal crystal grain structures, for example, with favorable binding lattice structures. Functionalization of implant surfaces may discourage bacterial formation and encouraging cell growth. The two may be interrelated as decreasing bacterial growth may reduce risk of infections which may hinder cell proliferation. It may also favor cell growth by reducing the amount of surface area occupied by bacteria. Further, selective functionalization of a surface of an implant 1090 may create changes in the hydrophilicity and hydrophobicity of a functionalized surface selectively favoring cell growth and disfavoring bacterial growth. An implant 1090 may comprise an implant portion, the implant portion may predominantly include sintered silicon nitride, for example, densely sintered silicon nitride, having a first chemical composition, the implant portion may include a machined functionalized surface operable to be integrated with mammalian tissue, and the functionalized surface may include a pattern resulting predominately from a laser process ablation or a laser spallation process. The implant portion may further have a second chemical composition on or adjacent the functionalized surface, and the second chemical composition may be a different chemical composition than the first chemical composition. The implant portion may include a depletion zone of the chemical composition and/or the availability of chemical substituents on and/or adjacent the functionalized surface that defines the second chemical composition when compared to the first chemical composition.

In one or more exemplary embodiments of the presently disclosed technology, unless the context requires otherwise, combinable with any other embodiment disclosed herein, a dental implant 1090 comprises a functionalized surface operational or otherwise configured to reduce to reduce gingival downgrowth. The functionalized surface may comprise a macroscale shape of spatial extension. The functionalized surface may comprise a machined microscale surface texture formed onto the macroscale shape. A microscale surface texture formed onto a macroscale shape of a transgingival portion of the dental implant 1090 may have multiple circumferential microgrooves and an average surface roughness suitable for tissue adhesion of adjacent human oral mucosa. Promoting soft tissue adhesion between the transgingival portion of the dental implant 1090 and the human oral mucosa may significantly reduce clinically the tendency for downgrowth of gingiva adjacent the dental implant 1090, which would compete, when the gingival downgrowth clinically covers an endosseous root portion of the dental implant 1090, with osseointegration of that endosseous root portion and would thereby undermine long term stability of the dental implant 1090, which could lead to implant failure. The microgrooves may have a spatial extension as shown and/or discussed, for example, with respect to the microscale surface texture 220 as depicted in FIG. 2. The microgrooves and/or an adjacent surface of the transgingival portion of the dental implant 1090 may have a nanoscale surface topography formed thereon, having an average surface roughness, Ra between 0.2 micrometer and 0.4 micrometer, for example about 0.3 micrometer. The microgrooves may be machined by a deterministic laser ablation process 1080. The surface roughness may be machined by a stochastic laser spallation process 1080. Polished surface qualities as part of a functionalized surface or adjacent a functionalized surface may be machined by a laser turning process.

In one or more exemplary embodiments of the presently disclosed technology, unless the context requires otherwise, combinable with any other embodiment disclosed herein, a dental implant 1090 comprises a root portion having a functionalized surface operational or otherwise configured for integration into or with human periodontal tissue. The functionalized surface may comprise a macroscale shape of spatial extension. The functionalized surface may comprise a machined microscale surface texture formed onto the macroscale shape. The root portion may include a mineral or a mineral aggregate. The root portion may include at least traces of natural or denaturized mammalian dentin.

In one or more exemplary embodiments of the presently disclosed technology, unless the context requires otherwise, combinable with any other embodiment disclosed herein, an implant 1090 comprises a functionalized surface operational or otherwise configured for mammalian tissue integration. The functionalized surface may include a plurality of machined micropores. An inner surface of the plurality of machined micropores may include a laser ablation pattern.

In one or more exemplary embodiments of the presently disclosed technology, unless the context requires otherwise, combinable with any other embodiment disclosed herein, an implant 1090 comprises a functionalized surface operational or otherwise configured for mammalian tissue integration. The functionalized surface may include a machined microscale texture having machined microscale undercuts. A surface of the machined microscale undercuts may include a laser ablation pattern.

In one or more exemplary embodiments of the presently disclosed technology, unless the context requires otherwise, combinable with any other embodiment disclosed herein, a medical device manufacturing system 1080 comprises a workpiece including a macroscale shape of spatial extension. The medical device manufacturing system 1080 may further comprise first computer numerical control data 1070 stored or storable on one or more non-transitory processor-readable memory 1030, 1056, and 1066 as computer-executable instructions or embodied in one or more non-transitory processor-readable computer data signals 1024, 1032, 1059, 1069, and 1070 as computer-executable instructions. A microscale surface texture may be formed onto the macroscale shape using the first computer numerical control data 1070. The medical device manufacturing system 1080 may further comprise a technical specification 1059, 1069 that identifies the workpiece as a semi-finished or finished product to become an implant 1090. The medical device manufacturing system 1080 may further comprise second computer numerical control data 1070 stored or storable on the one or more non-transitory processor-readable memory 1030, 1056, 1066 as the computer-executable instructions or embodied in the one or more non-transitory processor-readable computer data signals 1024, 1032, 1059, 1069, and 1070 as computer-executable instructions. The macroscale shape may correlate to the second computer numerical control data 1070. The medical device manufacturing system 1080 may further comprise clinical imaging data 1024, 1025 stored or storable on the one or more non-transitory processor-readable memory 1030, 1056, 1066 or embodied in one or more non-transitory processor-readable computer data signals 1024, 1032, 1059, 1069, and 1070. The clinical imaging data 1024, 1025 may be descriptive of an individual anatomical shape 1012 of a pre-identified patient 1010. The macroscale shape may correlate to the clinical imaging data 1024, 1025. The medical device manufacturing system 1080 may further comprise third computer numerical control data 1070 stored or storable on the one or more non-transitory processor-readable memory 1030, 1056, 1066 as the computer-executable instructions or embodied in one or more non-transitory processor-readable computer data signals 1024, 1032, 1059, 1069, and 1070 as computer-executable instructions. A nanoscale surface topography may be formed onto the microscale surface texture. The nanoscale surface topography may correlate to the third computer numerical control data 1070. The medical device manufacturing system 1080 may further comprise metrology measurement data stored or storable on the one or more non-transitory processor-readable memory 1030, 1056, and 1066. The metrology measurement data may be descriptive of a virtual surface of an at least two-dimensional extension having a macroscale resolution and a microscale resolution. The medical device manufacturing system 1080 may further comprise a laser system. The medical device manufacturing system 1080 may further comprise a first set of laser control parameters. The medical device manufacturing system 1080 may further comprise a second set of laser control parameters. A first numerical instruction may be operational or otherwise configured to operate the laser system responsive to the first set of laser control parameters and responsive to the first computer numerical control data 1070. A second numerical instruction may be operational or otherwise configured to operate the laser system responsive to the second set of laser control parameters and responsive to the second computer numerical control data 1070. The first set of laser parameters and the second set of laser parameters may be different laser parameters. The medical device manufacturing system 1080 may further comprise a set of laser control parameter. The medical device manufacturing system 1080 may further comprise an ultra-short-pulsed laser system which uses the set of laser control parameters to form the microscale surface texture onto the macroscale shape. The medical device manufacturing system 1080 may further comprise a XY galvanometer mirror scanner responsive to the first computer numerical control data 1070. The medical device manufacturing system 1080 may further comprise a Z shifter responsive to the first computer numerical control data 1070. The medical device manufacturing system 1080 may further comprise a linear X machine axis responsive to the first computer numerical control data 1070. The medical device manufacturing system 1080 may further comprise a linear Y machine axis responsive to the first computer numerical control data 1070. The medical device manufacturing system 1080 may further comprise a linear Z machine axis responsive to the first computer numerical control data 1070. The medical device manufacturing system 1080 may further comprise a rotary axis responsive to the first computer numerical control data 1070. The medical device manufacturing system 1080 may further comprise a swivel axis responsive to the first computer numerical control data 1070. The ultra-short-pulsed laser system and the set of laser control parameters may be operational or otherwise configured predominantly for laser ablation processing of the workpiece. The ultra-short-pulsed laser system and the set of laser control parameters may be operational or otherwise configured predominantly for laser ablation processing of the workpiece based on laser interferometry. The ultra-short-pulsed laser system and the set of laser control parameters may be operational or otherwise configured predominantly for laser spallation processing of the workpiece.

In one or more exemplary embodiments of the presently disclosed technology, unless the context requires otherwise, combinable with any other embodiment disclosed herein, a medical device manufacturing system 1080 comprises a laser system generating a laser beam operational or otherwise configured for laser ablation processing, a workpiece, and computer numerical control data 1070 stored or storable on one or more non-transitory processor-readable memory as computer-executable instructions or embodied in one or more non-transitory processor-readable computer data signals 1024, 1032, 1059, 1069, and 1070 as computer-executable instructions. The workpiece may include a macroscale shape of spatial extension. The computer numerical control data 1070 may be representative of a virtual differential laser ablation volume correlating to a virtual semi-finished shape of the workpiece and correlating to a corresponding virtual shape model of an implant. The macroscale shape may correlate to the virtual shape model of the implant. The medical device manufacturing system 1080 may further comprise a technical specification that identifies the workpiece as a semi-finished or finished product to become an implant 1090. The virtual semi-finished shape of the workpiece may have a virtual spatial macroscale extension that deviates from the virtual shape model of the implant 1090. The virtual semi-finished shape of the workpiece may represent a generic shape of spatial macroscale extension. The virtual shape model of the implant 1090 may represent a custom-shape of spatial macroscale extension. The virtual shape model of the implant 1090 may correlate to an individual anatomical shape 1012 of a pre-identified patient 1010. The virtual differential laser ablation volume may exceed about 5% of the volume of the workpiece or the virtual semi-finished shape of the workpiece. The medical device manufacturing system 1080 may further comprise an in-line metrology measurement instrument operational or otherwise configured to measure a shape of the workpiece. The virtual semi-finished shape of the workpiece may correlate to spatial metrology measurement data of a semi-finished shape of the workpiece. The medical device manufacturing system 1080 may further comprise a first galvanometer operable to control a first mirror deflecting the laser beam in response to the computer numerical control data 1070 in a first direction. The medical device manufacturing system 1080 may further comprise a second galvanometer operable to control a second mirror deflecting the laser beam in response to the computer numerical control data 1070 in a second direction different than first direction. The medical device manufacturing system 1080 may further comprise an optical system operable to focus the laser beam and gain an intensity profile of the laser beam such that the intensity profile exceeds at least partially an ablation threshold of the material of the workpiece. The medical device manufacturing system 1080 may further comprise an optical focus shifter, or a machine stage, operable to shift a focus of the laser beam in the direction of a main axis of the laser in relation to a surface of the workpiece. The medical device manufacturing system 1080 may further comprise a control unit. The laser system, the control unit, the first galvanometer, the second galvanometer, the optical system, and the focus shifter or the machine stage may be operational or otherwise configured to ablate a plurality of ablation layers of the workpiece. An ablation layer of the plurality of ablation layers may have a two-dimensional boundary or a three-dimensional boundary, and a layer thickness in the direction of the main axis of the laser beam, also referred to a patch. A plurality of patches may form an ablation blanket. Multiple ablation blankets may be stacked to form an ablation volume. The medical device manufacturing system 1080 may further comprise a rotational machine axis operable to rotationally position the workpiece, in relation to the spatial working range, responsive to the computer numerical control data 1070. The laser system, the control unit, the first galvanic actuator, the second galvanic actuator, the optical system, the focus shifter or the machine stage, and the rotational machine axis may be operational or otherwise configured to patch or map the plurality of ablation layers, responsive to the computer numerical control data 1070, onto a circumferential extension of the workpiece, thus machining a shape of the workpiece. The shape of the workpiece may correspond to the virtual shape model of the implant 1090. At least first two adjacent ablation layers of a first layer of the plurality of ablation layers may form a first joint or a first gap. At least second two adjacent ablation layers of a second layer of the plurality of ablation layers may form a second joint or a second gap. The laser system, the control unit, the first galvanic actuator, the second galvanic actuator, the optical system, the focus shifter or the machine stage, and the rotational machine axis may be operational or otherwise configured to patch or map the plurality of ablation layers responsive to the computer numerical control data 1070 so that an adjacent pair of joints or gaps including the first joint and the second joint, or the first gap and the second gap, are positioned so that a cumulative build-up of joints or gaps is avoided.

In one or more exemplary embodiments of the presently disclosed technology, unless the context requires otherwise, combinable with any other embodiment disclosed herein, a medical device manufacturing system 1080 comprises a laser system generating a laser beam operational or otherwise configured for laser ablation processing. The medical device manufacturing system 1080 may further comprise a workpiece. The medical device manufacturing system 1080 may further comprise a technical specification that identifies the workpiece as a semi-finished or finished product to become an implant 1090. The medical device manufacturing system 1080 may further comprise a trepanning optic unit based on rotating cylindrical lenses forming a helical deflection of the laser beam, a resulting trepanning laser beam having a main axis. A focused laser itself or a helically deflected laser beam may be operationally positioned tangentially or with a minimal secant to a rotating convex or round workpiece or segment of a workpiece to thereby ablate material predominately tangential to a circumferential segment of the workpiece. This process may be also referred to as “laser turning”. The term “CNC turning” can apply to technologies based on laser turning. The medical device manufacturing system 1080 may further comprise an optical system operable to focus the laser beam to thereby gain an intensity profile of the laser beam such that the intensity profile exceeds at least partially an ablation threshold of the material of the workpiece. The medical device manufacturing system 1080 may further comprise a rotational machine axis to rotate the workpiece. The medical device manufacturing system 1080 may further comprise a control unit. The medical device manufacturing system 1080 may further comprise computer numerical control data 1070 stored or storable on a non-transitory processor-readable memory as computer-executable instructions or embodied in one or more non-transitory processor-readable computer data signals 1024, 1032, 1059, 1069, and 1070 as computer-executable instructions. The workpiece may include a macroscale shape of spatial extension. The computer numerical control data 1070 may be representative of a virtual shape model of an implant 1090. The macroscale shape may correlate to the computer numerical control data 1070. The laser system, the control unit, the trepanning optic unit, the optical system, and the rotational machine axis may be operational or otherwise configured to ablate a plurality of ablation layers of the workpiece. An ablation layer of the plurality of ablation layers having a layer thickness substantially perpendicular to a direction of a main axis of the laser beam.

Without limiting the foregoing and unless the context requires otherwise, all features or other elements as disclosed herein with respect to FIG. 10 shall be deemed herewith disclosed in any combination with each other, in any partial combination with each other, and in any order; and shall be deemed herewith disclosed in combination with all features or other elements as disclosed herein with respect to FIGS. 1 to 9, and 11 to 14 in any combination, in any partial combination, and in any order. Without limiting the foregoing and unless the context requires otherwise, the implant 1090 referenced with respect to FIG. 10 may represent the same or similar implant, e.g., 100, or 150, the same or similar workpiece, e.g., 310, 520, 620, 755, 822, 922, or the same and similar like elements, respectively, as shown in and/or described with respect to FIGS. 1, 3, 5 to 9, 14, and other figures herein, respectively, and vice versa. Without limiting the foregoing and unless the context requires otherwise, the implant surface referenced with respect to FIG. 10 may represent the same or similar surfaces, e.g., 110, 112, 114, 116, 120, 160, 161, 164, 166, 170, 210, 400, 826, 868, or 924, or like elements as shown in and/or described with respect to FIGS. 1 to 4, 8 to 9, and other figures herein, respectively. Without limiting the foregoing and unless the context requires otherwise, the anatomical structures, the image data, the clinical data 1024, 1025, the patient 1010, the computer numerical control (CNC) data 1070, the computer data or computer data signals 1024, 1032, 1059, 1069, and 1070, the one or more processors 1057, and 1067, the computer hardware configurations 1055, and 1065, the personal computer (PC) or workstations 1050, and 1060, the one or more memory 1056, and 1066, the manufacturing facility, manufacturing systems, manufacturing equipment, or manufacturing methods 1080, the laser processes, the laser system, the software, computer program products, or software configurations 1055, and 1065, the processor-readable instructions, the specification 1090, the machine equipment, the local or wide-area network 1040, the database 1030, and the data storage system 1030, as each referenced with respect to FIG. 10 may represent the same or similar anatomical structures, the same or similar image data, the same or similar clinical data, the same or similar patient, the same or similar computer numerical control (CNC) data, computer data or computer data signals, the same or similar one or more processors, the same or similar computer hardware configurations, personal computer (PC) or workstation, the same or similar one or more memory, the same or similar manufacturing facility, manufacturing system, manufacturing equipment, or manufacturing methods, e.g., 330, 332, 500, 700, 534, 720, 730, 740, 750, and 760, the same or similar laser processes, e.g., 300, 540, 600, 820, 840, 860, 920, 940, and 960, the same or similar laser system, e.g., 500, the same or similar software, computer program products, or software configurations, the same or similar processor-readable instructions, the same or similar specification, the same or similar machine equipment, the same or similar local or wide-area network, the same or similar database, and the same or similar data storage system, or the same and similar like elements, respectively, as shown in and/or described with respect to FIGS. 1, to 3, 5 to 9, 14, and other figures herein, respectively, and vice versa. Without limiting the foregoing and unless the context requires otherwise, the machining, the manufacturing process 1080, and the various method steps referenced with respect to FIG. 10 may represent the same or similar the same or similar machining, the same or similar manufacturing process, e.g., 300, 540, 600, 612, 820, 840, 860, 930, 950, 979, and 1400, and the various same or similar method steps, e.g., 1410, 1420, 1430, 1440, 1450, or 1460, or the same or similar like elements, respectively, as described and/or shown with respect to FIGS. 1, 2, 14, and other figures herein, respectively, and vice versa.

In one or more exemplary embodiments of the presently disclosed technology, unless the context requires otherwise, combinable with any other embodiment disclosed herein, a method of designing and/or manufacturing a customized dental implant for a pre-identified patient comprises numerical machine-learning processes and/or artificial intelligence (AI) processes. Clinical images may include numerical representations of anatomical dental structures or elements of the pre-identified patient, for example, adjacent and opponent teeth, jawbone structures, gingiva, and/or temporomandibular joint (TMJ) structures, or portions thereof. The representations may be patient-specific, and may include one or more shapes of anatomical elements, one or more dimensional extension of anatomical elements, positions and/or inclinations of two or more anatomical elements in dimensional relation to each other, surface color information, and planar or volumetric density or intensity information. The representations may have limitations as the information of various tissues may be superimposed, not delineated, and/or not labeled. With other words, for example, various portions of the representation may relate to various anatomical elements or portions thereof without the availability of specific distinguishing or identifying information. Clinical images received in planar or volumetric density or intensity information, for example, CT or MRI images, may represent the outline of an anatomical element, or portions thereof, as a gradient of ambiguous, noisy or cluttered density or intensity information, without a clear delineation of its spatial extension. Computer program products, including a data set and a set of program instructions executable on one or more processors and computer systems may assist in the delineation of one or more spatial extensions of one or more anatomical element or portions thereof described in the numerical representations of a clinical image. The numerical generation of a virtual delineation or virtual outline of an anatomical element or a portion thereof as numerically represented in a clinical image is herein also referred as “surface reconstruction”. The identification or demarcation of an anatomical element or a portion thereof as numerically represented in a clinical image is herein also referred to as “labeling”. In this context and throughout this disclosure, the term “shape model” and derivative or similar words shall be understood as being generic to all possible meanings supported by the specification and by the words itself; the meaning shall, however, include herein, without limiting the foregoing and unless the context requires otherwise, a data set, for example, alone or in combination with a set of program instructions, that includes or is operational or otherwise configured to include at least a numerical statistical essence of trained data of virtual shapes of a plurality of like anatomical elements. The term “shape model” shall include the term “statistical shape models”. In this context and throughout this disclosure, the term “coupled shape model” and derivative or similar words shall be understood as being generic to all possible meanings supported by the specification and by the words itself; the meaning shall, however, include herein, without limiting the foregoing and unless the context requires otherwise, a data set, for example, alone or in combination with a set of program instructions, that includes or is operational or otherwise configured to include at least a first numerical statistical essence of trained data of virtual shapes of a first plurality of like first anatomical elements, a second numerical statistical essence of trained data of virtual shapes of a second plurality of like second anatomical elements, and a third numerical statistical essence of trained data of virtual dimensional positions and/or virtual dimensional inclinations of the first plurality of like first anatomical elements in geometric positional relation to the second plurality of like second anatomical elements or in geometric positional relation to common reference frame or coordinate system. The term “coupled shape model” shall include the term “statistical coupled shape models”. The term virtual orientation may include a virtual dimensional position and/or a virtual dimensional inclination. A trained shape model may be descriptive of a statistical dental anatomy model. A trained coupled shape model may be descriptive of a labeled statistical dental anatomy model. The statistical dental anatomy model may include one or more statistical dental anatomy element shape models. The statistical dental anatomy model may include a plurality of trained constraint models of virtual statistical shape variabilities. A statistical dental anatomy element shape model may correspond to a statistical dental anatomy element orientation model that may include a trained orientation constraint model of virtual statistical orientation variability.

A method of designing and/or manufacturing a customized dental implant for a pre-identified patient may comprise obtaining one or more clinical images including one or more numerical representations of one or more dental anatomical elements of the pre-identified patient and deriving a design or updating a design of the dental implant responsive to the one or more clinical images to thereby create or derive a patient-specific custom-shaped design of the dental implant. The method may further comprise reconstructing one or more virtual surfaces of the one or more anatomical elements using a trained shape model or a trained coupled shape model to thereby create one or more patient-specific anatomical shape models. The method may further comprise labeling the one or more anatomical elements using a trained shape model or a trained coupled shape model to thereby create or update one or more patient-specific labeled anatomical shape models. The method may further comprise determining one or more orientations of the one or more anatomical elements using a trained coupled shape model to thereby create or update one or more patient-specific anatomical orientation models of the one or more patient-specific anatomical shape models. The customization or the custom-shaping of a patient-specific dental implant, or and derivative or similar words shall include by way of example and not limitation, the shaping of a surface portion of the dental implant to correlate to or to match at least a portion of a dental anatomical element of the pre-identified patient, for example, at least a portion of a tooth of the pre-identified patient, for example, a crown, a transgingival portion or a root, at least a portion of an alveolar socket of the pre-identified patient or an planned implant bed, or at least a portion of a bone crest or an outer or an inner gingival margin.

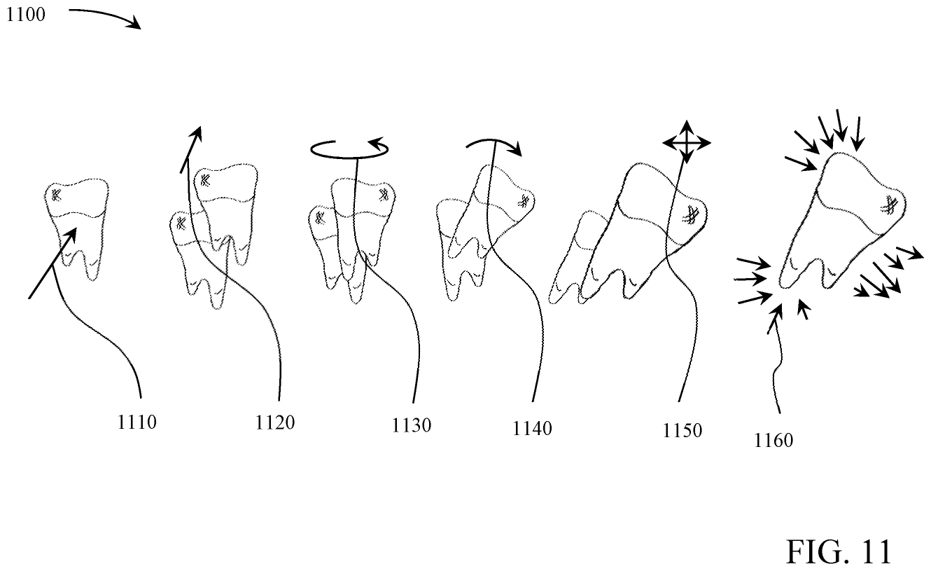

FIG. 11 shows an illustration of exemplary embodiments of a virtual shape adaptation process, in accordance with one or more embodiments of the presently disclosed technology, and, unless the context requires otherwise, combinable with any other embodiment disclosed herein.

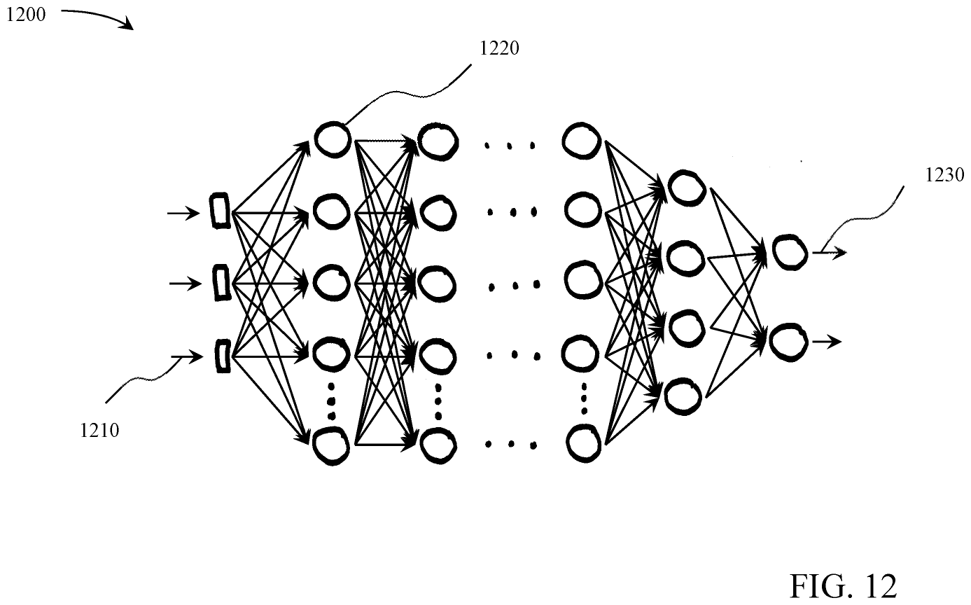

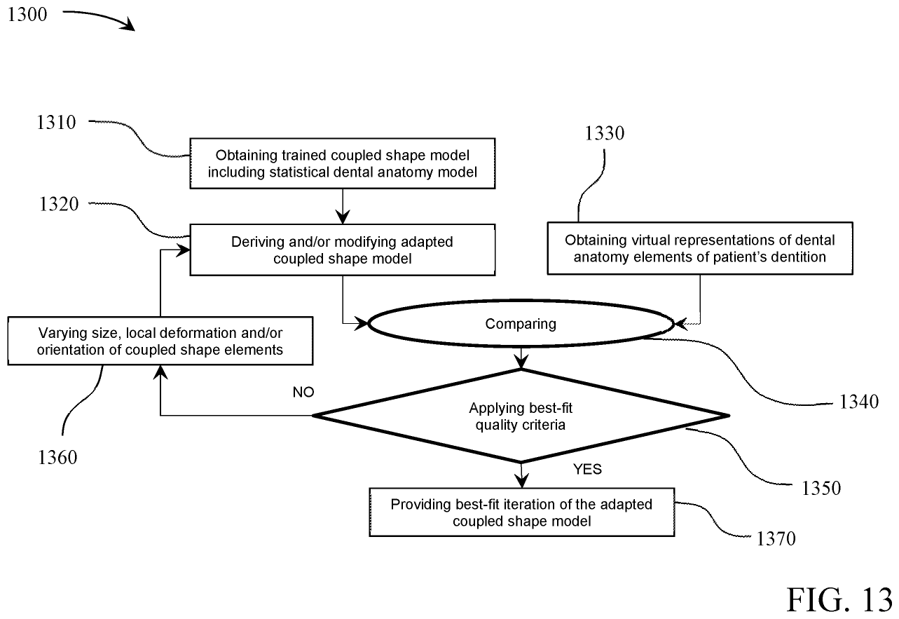

In one or more exemplary embodiments of the presently disclosed technology, unless the context requires otherwise, combinable with any other embodiment disclosed herein, a method of designing and/or manufacturing a customized dental implant for a pre-identified patient comprises using a trained statistical coupled shape model. The method may comprise a process 1100 including receiving 1110 a virtual model, for example, a virtual dental anatomical model, a virtual shape element, and/or a virtual coupled shape element, numerically varying 1120, 1130, 1140, 1150, and 1160 the virtual model, for example a virtual tooth, or portions thereof. The process 1100 may include orienting 1120, 1130, and 1140 the virtual model, sizing 1150 the virtual model, and/or locally deforming 1160 the virtual model. The process step orienting 1120, 1130, and 1140 the virtual model may include a linear positioning 1120 of the virtual model, and/or a rotational positioning 1130, and 1140 of the virtual model. The sizing 1150 of the virtual model may be uniform or directional. The virtual model may be a two-dimensional, a three-dimensional model, or a four-dimensional model. The process 1100, or portions thereof, may be performed in a two-dimensional, in a three-dimensional and/or a four-dimensional space. The virtual model may be numerically represented in shape spaces and/or in intensity spaces. The local deformation process may use cubic B-spline based or other free form deformations (FFD). The method may employ the process 1100 as part of an iterative numerical optimization process, as, for example, depicted and/or described with respect to FIG. 13. The iterative numerical optimization process may use a statistical technique for reducing the dimensionality of a dataset, for example, a principal component analysis (PCA). The iterative numerical optimization process may use a statistical regression analysis, for example, a linear regression analysis, or a method of ordinary least squares. The iterative numerical optimization process may use or may be employed by an artificial neuronal network as, for example, depicted and/or described with respect to FIG. 12.

FIG. 12 shows a schematic block diagram illustrating an exemplary embodiment of a neuronal network and a principal data flow in accordance with one or more embodiments of the presently disclosed technology, and, unless the context requires otherwise, combinable with any other embodiment disclosed herein.

In one or more exemplary embodiments of the presently disclosed technology, unless the context requires otherwise, combinable with any other embodiment disclosed herein, a method of designing and/or manufacturing a customized dental implant for a pre-identified patient may employ a pre-parameterized or hyper-parametrized artificial neuronal network 1200, having, for example, a pre-determined number of input parameters or numerical input objects 1210, a pre-determined number of rows and a pre-determined number of columns of artificial neuronal nodes or neurons 1220, and/or a pre-determined number of output parameters or numerical result objects 1230 independent of the number of training examples used for teaching the artificial neuronal network 1200. A plurality of artificial neuronal nodes or neurons 1220 may be arranged in a matrix, or an otherwise operational or otherwise configured layer. The method may employ an artificial neuronal network 1200, having, for example, a variable number of input parameters or numerical input objects 1210, a variable pre-determined number of rows and a variable number of columns of artificial neuronal nodes or neurons 1220, and/or a variable number of output parameters or numerical result objects 1230 independent of the number of training examples used for teaching the artificial neuronal network 1200. One or more of these variable numbers may be determined or adjusted by the computation using the artificial neuronal network 1200. The values of parameters may be derived via learning the artificial neuronal network 1200. Learning may include the adaptation of the artificial neuronal network 1200 to improve an accuracy of a result, minimizing errors.

FIG. 13 shows an illustration of an exemplary embodiment of a flow diagram for a plurality of method steps for deriving or modifying iteratively an adapted coupled shape model descriptive of a dental anatomy in accordance with one or more embodiments of the presently disclosed technology.

Flow diagram 1300 shows one or more other exemplary embodiments, unless the context requires otherwise, combinable with any other embodiment disclosed herein, of a method for deriving or modifying iteratively an adapted coupled shape model descriptive of a dental anatomy in the context of designing custom-shaped dental implants. The method depicted in diagram 1300 can comprise: method step 1310 of obtaining a trained coupled shape model including a statistical dental anatomy model; method step 1320 of deriving and/or modifying an adapted coupled shape model; method step 1330 of obtaining one or more virtual representations of dental anatomy elements of a patient's dentition; method step 1340 of comparing a fit of the adapted coupled shape model with the one or more virtual representations of the dental anatomy elements; method step 1350 of applying a best-fit criteria to the results of the comparison of method step 1340 including a “YES” or “NO” decision step indicating the acceptance of the iteratively derived and/or modified adapted coupled shape model continuing to step 1370 or, alternatively, further iterating and optimizing the coupled shape elements included in the adapted coupled shape model continuing to method step 1360; method step 1360 of varying size, local deformation and/or orientation of coupled shape elements included in the adapted coupled shape model to thereby modify the adapted coupled shape model in method step 1320; and method step 1370 of providing a best-fit iteration of the adapted coupled shape model to subsequent processing steps.

The method steps 1310, 1320, 1330, 1340, 1350, 1360, and 1370 may be combined in any order and in any partial combination in any order.

In one or more exemplary embodiments of the presently disclosed technology, unless the context requires otherwise, combinable with any other embodiment disclosed herein, a method of manufacturing a customized dental implant for a pre-identified patient comprises obtaining a proposed specification of the dental implant. The dental implant may include an endosseous root portion and an occlusal facing portion operational or otherwise configured to receive a dental prosthesis. The method may further comprise obtaining a trained shape model. The trained shape model may be descriptive of a statistical dental anatomy model. The statistical dental anatomy model may include one or more statistical dental anatomy element shape models. The method may further comprise obtaining a data set including one or more virtual representations of one or more dental anatomy elements of a dentition of the patient. The method may further comprise adapting at least a portion of the trained shape model to best-fit the one or more virtual representations to thereby form an adapted shape model. The method may further comprise updating the proposed specification of the dental implant responsive to the adapted shape model to thereby form an updated specification. The method may further comprise machining the dental implant at least partially responsive to the updated specification so that a surface of the dental implant at least partially correlates to the adapted shape model. The data set may include one or more two-dimensional images representative of the one or more dental anatomy elements of the dentition of the patient. A two-dimensional image of the one or more two-dimensional images may include a plurality of pixels having assigned gradual intensity values. The two-dimensional image of the one or more two-dimensional images is a video frame, a picture, a two-dimensional image generated by an intraoral scanner, a two-dimensional array, or an X-ray image. The data set may include one or more two-dimensional images representative of the one or more dental anatomy elements of the dentition of the patient. A two-dimensional image of the one or more two-dimensional images may be a two-dimensional point cloud, a two-dimensional mesh, or a two-dimensional shape model. The data set may include one or more three-dimensional images of the one or more dental anatomy elements of the dentition of the patient. A three-dimensional image of the one or more three-dimensional images may include a plurality of voxels having assigned gradual intensity values. The three-dimensional image of the one or more three-dimensional images may be a CT, a cone beam CT, an MRI image, a three-dimensional X-ray, a frame of a dynamic three-dimensional model, a three-dimensional frame generated by an intraoral scanner, or a three-dimensional array. The data set may include one or more three-dimensional images of the one or more dental anatomy elements of the dentition of the patient. A three-dimensional image of the one or more three-dimensional images may be a three-dimensional point cloud, a three-dimensional mesh, a three-dimensional surface scan, or a three-dimensional shape model. The one or more virtual representations of the one or more dental anatomy elements embodied in the data set may be unlabeled. The adapted shape model may include one or more labeled virtual dental anatomy shape elements. A labeled virtual dental shape anatomy element of the one or more labeled virtual dental shape anatomy elements may include a numerical two-dimensional or there-dimensional surface reconstruction of a corresponding dental anatomy element of the one or more dental anatomy elements. A dental anatomy element of the one or more dental anatomy elements may include at least one of a tooth, a portion of the tooth, an alveolar socket, a portion of the alveolar socket, a gingival margin, or a portion of the gingival margin. A labeled virtual dental shape anatomy element of the one or more labeled virtual dental shape anatomy elements may be associated with a reference to a label corresponding to a dental tooth numbering scheme. A statistical dental anatomy element shape model of the one or more statistical dental anatomy element shape models includes a plurality of trained constraint models of virtual statistical shape variabilities. The method may further comprise an iterative numerical optimization process having one or more steps including: varying a virtual size of a virtual shape of a statistical dental anatomy element shape model of the one or more statistical dental anatomy element shape models within at least one virtual size constraint included in the plurality of trained constraint models of virtual statistical shape variabilities; varying a virtual local deformation of a virtual shape of the statistical dental anatomy element shape model within at least one virtual deformation constraint included in the plurality of trained constraint models of virtual statistical shape variabilities; or calculating a quality function. The updated proposed specification may include at least one virtual three-dimensional design model representing at least a portion of the dental implant selected from a group including at least two of: an abutment portion, an occlusal portion, a preparation post to receive a crown, a preparation post to receive a bridge, a preparation post to receive a prosthetic element, a transgingival portion, an implant neck, an endosseous portion, a root portion, an interface between the abutment portion and the endosseous portion, or a root-analogue portion. The trained shape model may be a multi-dimensional parametrized model. A statistical dental anatomy element shape model of the one or more of statistical dental anatomy element shape models may use a numerical structure including at least one of: a point distribution model, a principal component analysis, a vector array, a two-dimensional point cloud, a two-dimensional surface mesh, or a three-dimensional surface mesh. A computer program product stored or storable on a non-transitory processor-readable memory as executable instructions for executing, on a computer system, a computer process may embody one or more of the aforementioned method steps. A display of an electronic device visualizing an image output derived from a computer process may embody one or more of the aforementioned method steps. A method may teach a trained shape model as described above.

In one or more exemplary embodiments of the presently disclosed technology, unless the context requires otherwise, combinable with any other embodiment disclosed herein, a method of manufacturing a customized dental implant for a pre-identified patient comprises obtaining a proposed specification of the dental implant, the dental implant including an endosseous root portion and an occlusal facing portion or operational or otherwise configured to receive a dental prosthesis. The method may further comprise obtaining a trained coupled shape model. The trained coupled shape model may be descriptive of a statistical dental anatomy model. The statistical dental anatomy model may include a plurality of labeled statistical dental anatomy element shape models and a plurality of corresponding statistical dental anatomy element orientation models. The method may further comprise obtaining a data set including one or more virtual representations of a plurality of dental anatomy elements of a dentition of the patient. The method may further comprise adapting at least a portion of the trained coupled shape model to best-fit the one or more virtual representations to thereby form an adapted coupled shape model. The method may further comprise updating the proposed specification of the dental implant responsive to the adapted coupled shape model to thereby form an updated specification. The method may further comprise machining the dental implant at least partially responsive to the updated specification so that a surface of the dental implant at least partially correlates to the adapted shape model. The data set may include one or more two-dimensional images representative of the plurality of dental anatomy elements of the dentition of the patient, a two-dimensional image of the one or more two-dimensional images comprising a plurality of pixels having assigned gradual intensity values. The two-dimensional image of the one or more two-dimensional images may be a video frame, a picture, a two-dimensional image generated by an intraoral scanner, a two-dimensional array, or an X-ray image. The data set can include one or more two-dimensional images representative of the plurality of dental anatomy elements of the dentition of the patient. A two-dimensional image of the one or more two-dimensional images may be a two-dimensional point cloud, a two-dimensional mesh, a two-dimensional shape model, or a two-dimensional coupled shape model. The data set may include one or more three-dimensional images of the plurality of dental anatomy elements of the dentition of the patient, a three-dimensional image of the one or more three-dimensional images includes a plurality of voxels having assigned gradual intensity values. The three-dimensional image of the one or more three-dimensional images may be CT, a cone beam CT, an MRI image, a three-dimensional X-ray, a frame of a dynamic three-dimensional model, a three-dimensional frame generated by an intraoral scanner, or a three-dimensional array. The data set may include one or more three-dimensional images of the plurality of dental anatomy elements of the dentition of the patient. A three-dimensional image of the one or more three-dimensional images may be a three-dimensional point cloud, a three-dimensional mesh, a three-dimensional surface scan, or a three-dimensional coupled shape model. The one or more virtual representations of the plurality of dental anatomy elements embodied in the data set may be unlabeled. The adapted shape model may include one or more labeled virtual dental anatomy shape elements. A labeled virtual dental shape anatomy element of the one or more labeled virtual dental shape anatomy elements may include a numerical two-dimensional or three-dimensional surface reconstruction of a corresponding dental anatomy element of the one or more dental anatomy elements. A dental anatomy element of the one or more dental anatomy elements may include at least one of a tooth, a portion of a tooth, an alveolar socket, a portion of the alveolar socket, a gingival margin, or a portion of the gingival margin. A labeled virtual dental shape anatomy element of the one or more labeled virtual dental shape anatomy elements may be associated with a reference to a label corresponding to a dental tooth numbering scheme. A labeled statistical dental anatomy element shape model of the plurality of labeled statistical dental anatomy element shape models may include a plurality of trained shape constraint models of virtual statistical shape variabilities. A corresponding statistical dental anatomy element orientation model of the plurality of corresponding statistical dental anatomy element orientation models may include a trained orientation constraint model of virtual statistical orientation variability. The method step adapting may further include performing an iterative numerical optimization process having one or more steps including: varying a virtual size of a virtual shape of a labeled statistical dental anatomy element shape model of the plurality of labeled statistical dental anatomy element shape models within at least one virtual size constraint included in the plurality of trained shape constraint models of virtual statistical shape variabilities; varying a virtual local deformation of a virtual shape of the labeled statistical dental anatomy element shape model within at least one virtual deformation constraint included in the plurality of trained shape constraint models of virtual statistical shape variabilities; or varying a virtual orientation of a virtual shape of the labeled statistical dental anatomy element shape model within at least one virtual orientation constraint included in the trained orientation constraint model of virtual statistical orientation variability. The iterative optimization process may further include calculating a quality function. The updated proposed specification may include at least one virtual three-dimensional design model representing at least a portion of the dental implant including at least one of: an abutment portion, an occlusal portion, a preparation post to receive a crown, a preparation post to receive a bridge, a preparation post to receive a prosthetic element, a transgingival portion, an implant neck, an endosseous portion, a root portion, an interface between the abutment portion and the endosseous portion, or a root-analogue portion. The trained coupled shape model may be a multi-dimensional parametrized model including at least one of: a static two-dimensional model, a dynamic two-dimensional model, a three-dimensional model, or a dynamic three-dimensional model. A labeled statistical dental anatomy element shape model of the plurality of labeled statistical dental anatomy element shape models may use at least one numerical structure being at least one of: a point distribution model, a principal component analysis, a vector array, a two-dimensional point cloud, a two-dimensional surface mesh, or a three-dimensional surface mesh. A computer program product stored or storable on a non-transitory processor-readable memory as executable instructions for executing, on a computer system, a computer process may embody one or more of the aforementioned method steps. A display of an electronic device visualizing an image output derived from a computer process may embody one or more of the aforementioned method steps. A method may teach a trained coupled shape model as described above.

In one or more exemplary embodiments of the presently disclosed technology, unless the context requires otherwise, combinable with any other embodiment disclosed herein, a method of teaching a dental anatomy machine learning model comprises obtaining one or more individual exemplary dental anatomy models descriptive of one or more individual exemplary virtual dental anatomy shape elements. The method may further comprise obtaining a trainable or trained shape model, the trainable or trained shape model is descriptive of a statistical dental anatomy model, the statistical dental anatomy model includes one or more statistical dental anatomy element shape models. The method may further comprise updating the trainable or trained shape model responsive to the one or more individual exemplary dental anatomy models to thereby form an updated trained shape model. A statistical dental anatomy element shape model of the one or more statistical dental anatomy element shape models may include a plurality of corresponding trained constraint models of virtual statistical shape variabilities. The method step of updating the trainable or trained shape model may include updating, for the one or more statistical dental anatomy element shape models, the plurality of corresponding trained constraint models of virtual statistical shape variabilities responsive to a shape variability of the one or more individual exemplary virtual dental anatomy shape elements.

In one or more exemplary embodiments of the presently disclosed technology, unless the context requires otherwise, combinable with any other embodiment disclosed herein, a method of teaching a dental anatomy machine learning model comprises obtaining one or more individual exemplary dental anatomy models descriptive of a plurality of individual exemplary labeled virtual dental anatomy shape elements and corresponding exemplary virtual relative orientations. The method may further comprise obtaining a trainable or trained coupled shape model, the trainable or trained coupled shape model is descriptive of a statistical dental anatomy model, the statistical dental anatomy model includes a plurality of labeled statistical dental anatomy element shape models and a plurality of corresponding statistical orientation models. The method may further comprise updating the trainable or trained coupled shape model responsive to the one or more individual exemplary dental anatomy models to thereby form an updated trained coupled shape model. A labeled statistical dental anatomy element shape model of the plurality of labeled statistical dental anatomy element shape models may include a plurality of corresponding trained shape constraint models of virtual statistical shape variabilities. The method step of updating the trainable or trained coupled shape model may include updating, for the plurality of labeled statistical dental anatomy element shape models, the plurality of corresponding trained shape constraint models of virtual statistical shape variabilities responsive to a shape variability of the one or more individual exemplary labeled virtual dental anatomy shape elements. A corresponding statistical orientation model of the plurality of corresponding statistical orientation models may include a trained orientation constraint model of virtual statistical orientation variability. The method step of updating the trainable or trained coupled shape model may include updating, for a labeled statistical dental anatomy element shape model of the plurality of labeled statistical dental anatomy element shape models, the trained orientation constraint model of virtual statistical orientation variability responsive to an orientation variability of the plurality of the plurality of corresponding statistical orientation models.

Without limiting the foregoing and unless the context requires otherwise, all features or other elements as disclosed herein with respect to FIGS. 11, 12, and/or 13 shall be deemed herewith disclosed in any combination with each other, in any partial combination with each other, and in any order; and shall be deemed herewith disclosed in combination with all features or other elements as disclosed herein with respect to FIGS. 1 to 14, as applicable, in any combination, in any partial combination, and in any order. Without limiting the foregoing and unless the context requires otherwise, all features, other elements, and terms as disclosed herein with respect to FIGS. 11, 12, and/or 13 may represent the same or similar like features, other the same or similar like elements, and the same or similar like terms, respectively, as shown in and/or described with respect to FIGS. 1 to 14, as applicable, and other figures herein, respectively, and vice versa.

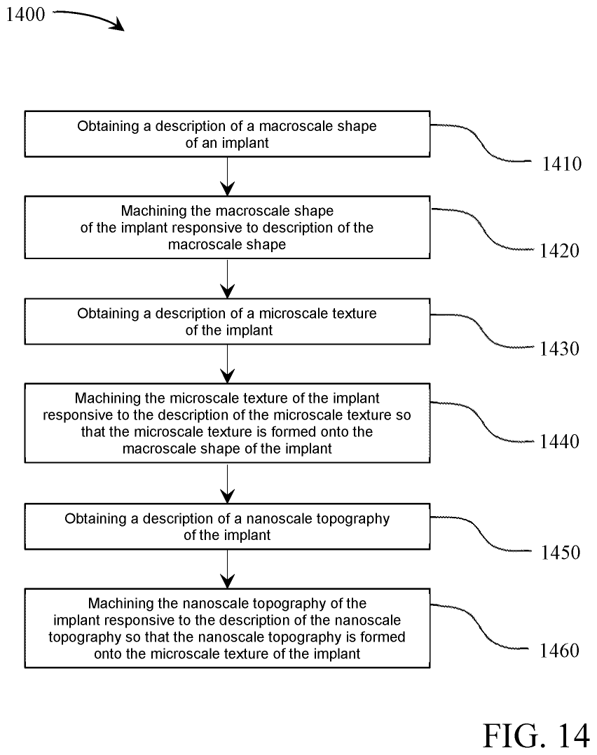

FIG. 14 shows an illustration of an exemplary embodiment of a flow diagram for a plurality of method steps for shaping and functionalizing an implant in accordance with one or more embodiments of the presently disclosed technology, and, unless the context requires otherwise, combinable with any other embodiment disclosed herein.

Flow diagram 1400 shows one or more other exemplary embodiments, unless the context requires otherwise, combinable with any other embodiment disclosed herein, of a method of manufacturing an implant, for example, for shaping and functionalizing an implant. The method depicted in diagram 1400 can comprise: method step 1410 of obtaining a description of a macroscale shape of an implant, for example, a net or near net shape; method step 1420 of machining the macroscale shape, for example, a net or near net shape, of the implant responsive to description of the macroscale shape; method step 1430 of obtaining a description of a microscale texture of the implant; method step 1440 of machining the microscale texture of the implant responsive to the description of the microscale texture so that the microscale texture is formed onto the macroscale shape of the implant; method step 1450 of obtaining a description of a nanoscale topography; and method step 1460 of machining the nanoscale topography of the implant responsive to the description of the nanoscale topography so that the nanoscale topography is formed onto the microscale texture of the implant. The method steps 1410, 1420, 1430, 1440, 1450, and 1460 may be combined in any order and in any partial combination in any order. For example, a flow diagram 1400 may comprise: method step 1430 of obtaining a description of a microscale texture of the implant; method step 1440 of machining the microscale texture of the implant responsive to the description of the microscale texture so that the microscale texture is formed onto a macroscale shape of the implant; method step 1450 of obtaining a description of a nanoscale topography; and method step 1460 of machining the nanoscale topography of the implant responsive to the description of the nanoscale topography so that the nanoscale topography is formed onto the microscale texture of the implant. For example, a flow diagram 1400 may comprise: method step 1410 of obtaining a description of a macroscale shape of an implant; method step 1420 of machining the macroscale shape of the implant responsive to description of the macroscale shape; method step 1430 of obtaining a description of a microscale texture of the implant; method step 1440 of machining the microscale texture of the implant responsive to the description of the microscale texture so that the microscale texture is formed onto the macroscale shape of the implant. For example, a flow diagram 1400 may comprise: method step 1410 of obtaining a description of a macroscale shape of an implant; method step 1420 of machining the macroscale shape of the implant responsive to description of the macroscale shape; method step 1450 of obtaining a description of a nanoscale topography; method step 1460 of machining the nanoscale topography of the implant responsive to the description of the nanoscale topography so that the nanoscale topography is formed onto the macroscale shape of the implant. The term “machining” in the context of the method steps 1420, 1440, and 1460 shall have the meaning as referenced with respect to FIG. 10. Method step 1420 may utilize laser ablation processes. Method step 1440 may utilize laser ablation processes, laser spallation processes, and/or other stochastic laser material removal technologies. Method step 1460 may utilize laser ablation processes, laser spallation processes, other stochastic laser material removal technologies, and/or laser process based on laser interferometry. In the context of the method steps 1410, 1430, and 1450 and throughout this disclosure, the term “description” and derivative or similar words shall be understood as being generic to all possible meanings supported by the specification and by the words itself; the meaning shall, however, include herein, without limiting the foregoing and unless the context requires otherwise, computer aided design (CAD) data, computer aided manufacturing (CAM) data, computerized numerical control (CNC) data, two-dimensional virtual models, three-dimensional virtual models, four-dimensional virtual models, and/or grayscale texture patterns. By way of example, and not limitation, a description may include a technical specification of the feature of interest. A description may include a virtual model of a macroscale shape, and a machined macroscale shape of the implant may correlate, for example, within manufacturing tolerances match, the virtual model of the macroscale shape. A description may include a virtual model of a microscale texture shape, and a machined microscale texture of the implant may correlate, for example, within manufacturing tolerances match, the virtual model of the microscale texture. A description may include a virtual model of a nanoscale topography, and a machined nanoscale topography of the implant may correlate, for example, within manufacturing tolerances match, the virtual model of the nanoscale topography. A method of manufacturing an implant 1400 may comprise a computer aided design (CAD) process and/or a computer aided manufacturing (CAM) process that includes, for example, receiving a virtual target shape representing, for example, a shape of an implant, receiving a first virtual shape representing, for example, a shape of a workpiece, deriving therefrom a second virtual shape correlating to the first virtual shape and having a constant distance to the first shape, for example 2 micrometer, deriving a third virtual shape correlating to the first virtual shape and having a constant distance to the first shape, for example 4 micrometer, deriving a fourth virtual shape correlating to the first virtual shape and having a constant distance to the first shape, for example 6 micrometer, deriving a fifth virtual shape correlating to the first virtual shape and having a constant distance to the first shape, for example 8 micrometer, and so on, until a volume between the first virtual shape and the virtual target shape is divided into a plurality of intermediate virtual shapes parallel to the first virtual shape, the plurality of intermediate shapes, to include, for example, the second virtual shape, the third virtual shape, the fourth virtual shape, the fifths virtual shape, and so on. The aforementioned computer aided design (CAD) process and/or a computer aided manufacturing (CAM) process may further include reducing the dimensional extension of the intermediate virtual shapes of the plurality of intermediate virtual shapes responsive to the virtual target shape, so that the intermediate virtual shapes not intersecting the virtual target shape. This process step of reducing the dimensional extension of the intermediate virtual shapes may include a use of a Boolean numerical modelling operation between an intermediate virtual shape, for example the fifth virtual shape, and the virtual target shape. The Boolean numerical modelling operation may include an intersection operation. The Boolean numerical modelling operation may further include a union operation, a difference operation, and/or a symmetric difference operation. A Boolean numerical modelling operation may be operated on meshes. The aforementioned virtual shapes may be two-dimensional or three-dimensional. The shapes may have a generic shape or a custom shape. An intermediate virtual shape, also referred to as a layer or a blanket, may then be broken up in tiles or patches as shown in and/or discussed, for example, with respect to FIG. 4. The differential volume discussed in reference to FIG. 4 may be represented by a plurality of intermediate virtual shapes that does not intersect the virtual target shape. A plurality of intermediate virtual shapes that does not intersect the virtual target shape may include a plurality of parallel intermediate virtual shapes. Creating a plurality of intermediate virtual shapes may be also referred to as “slicing”. A layer-by-layer laser ablation process as discussed, for example, with respect to FIGS. 4 to 7, and 10 may be made in response to the plurality of intermediate virtual shapes that does not intersect the virtual target shape. A method of manufacturing an implant 1400 may comprise a computer aided design (CAD) process and/or a computer aided manufacturing (CAM) process that includes, for example, a numerical process of wrapping or mapping a two-dimensional or three-dimensional texture onto or around a three-dimensional shape. A method to manufacture a dental implant may comprise obtaining a specification of the dental implant, the specification of the dental implant may include a numerical description of a three-dimensional shape of the dental implant. The method may further comprise obtaining a specification of a workpiece designated to become an implant, the specification of the workpiece may include a numerical description of a three-dimensional shape of the workpiece. The method may further comprise obtaining a virtual spatial dimensional relationship between the three-dimensional shape of the dental implant and the three-dimensional shape of the workpiece relating, for example, to a three-dimensional virtual space. The method may further comprise deriving a virtual volume based on the three-dimensional shape of the dental implant, based on the three-dimensional shape of the workpiece, and, for example, based on the virtual spatial dimensional relationship. The method may further comprise slicing the virtual volume to thereby form a plurality of virtual ablation layers, a virtual ablation layer of the plurality of virtual ablation layers may have a spatial extension not intersecting with the three-dimensional shape of the dental implant, for example, within the three-dimensional virtual space, and the virtual ablation layer may have a shape predominately parallel to the three-dimensional shape of the workpiece. The method may further comprise deriving a plurality of patches from the virtual ablation layer. The method may further comprise deriving computer numerical control (CNC) data stored or storable on one or more non-transitory processor-readable memory as first computer-executable instructions from the virtual ablation layer. The method may further comprise functionalizing a surface of the dental implant using a laser process controlled by the computer numerical control (CNC) data.

In one or more exemplary embodiments of the presently disclosed technology, unless the context requires otherwise, combinable with any other embodiment disclosed herein, a method of manufacturing an implant comprises obtaining a specification of the implant. The implant may include a first functionalized surface operational or otherwise configured for integration into or with a first type of mammalian tissue. The first functionalized surface may include a first macroscale shape and a first microscale texture formed onto the first macroscale shape. The specification may include a description of the first microscale texture. The method may further comprise machining the first microscale texture responsive to the description of the first microscale texture. The specification may further include a description of the first macroscale shape. The method may further comprise machining the first macroscale shape responsive to the description of the first macroscale shape. The first functional surface may further include a first nanoscale topography formed onto the first microscale texture. The specification may further include a description of the first nanoscale topography. The method may further comprise machining the first nanoscale topography responsive to the description of the first nanoscale topography. The method may further comprise applying a first coating, the first coating includes the first nanoscale topography. The method may further comprise removing at least partially the first coating, for example by using a laser ablation process. The implant may further include a second functionalized surface operational or otherwise configured for integration into or with a second type of mammalian tissue. The second functionalized surface may include a second macroscale shape and a second microscale texture formed onto the second macroscale shape. The specification may further include a description of the second microscale texture. The method may further comprise machining the second microscale texture responsive to the description of the second microscale texture. The second type of mammalian tissue and the first type of mammalian tissue may be different tissue types. The second microscale texture and the first microscale texture are different texture types. The specification may further include a description of the second macroscale shape. The method may further comprise machining the second macroscale shape responsive to the description of the second macroscale shape. The second macroscale shape and the first macroscale shape may be different shapes. The second functional surface may further include a second nanoscale topography formed onto the first microscale texture. The second nanoscale topography and the first nanoscale topography may be different nanoscale topographies. The specification may further include a description of the second nanoscale topography. The method may further comprise machining the second nanoscale topography responsive to the description of the second nanoscale topography. The method may further comprise applying a second coating, the second coating includes the second nanoscale topography. The method may further comprise removing at least partially the second coating, for example, using a laser ablation process. The first functionalized surface may include a pattern resulting predominately from a laser process based on laser interferometry, a laser ablation process, or a laser spallation process. The first functionalized surface may include a coating resulting from chemical or physical vapor deposition process. The first nanoscale surface topography may be at least partially formed by at least one of grains, crystals, crystallites, polymorphic aggregates, nano-pores, or amorphic aggregates included in a substrate material or a coating material of the implant. The implant may be operational or otherwise configured for dental implantation. The description of the first macroscale shape may be at least partially representative of an anatomical shape correlating to a shape of a root of a tooth of a pre-identified patient, or to a shape of an alveolar socket of the pre-identified patient. The description of the first macroscale shape may be at least partially representative of an anatomical shape correlating to a crown of a pre-identified patient, a transmucosal portion of a tooth of the pre-identified patient, a gingival margin of the pre-identified patient, or a bone crest adjacent an alveolar socket of the pre-identified patient. The method step of machining the first macroscale shape may use a process of customization. The first macroscale shape of the implant may correlate at least partially to the anatomical shape.

In one or more exemplary embodiments of the presently disclosed technology, unless the context requires otherwise, combinable with any other embodiment disclosed herein, a method of manufacturing an implant comprise obtaining a specification of the implant. The implant may include a functionalized surface operational or otherwise configured for integration into or with a type of mammalian tissue. The functionalized surface may include a macroscale shape and a microscale texture formed onto the macroscale shape, and a nanoscale topography formed onto the microscale texture, the specification includes a description of the nanoscale topography. The method may further comprise machining the nanoscale topography responsive to the description of the nanoscale topography. The nanoscale topography may include a pattern predominately resulting from a laser process based on laser interferometry, a laser ablation process, or a laser spallation process. A method of manufacturing a dental implant may comprise obtaining a specification of the dental implant. The dental implant may include a functionalized surface operational or otherwise configured for integration into or with a periodontal ligament structure. The functionalized surface may include a macroscale shape and a microscale texture formed onto the macroscale shape. The specification may include a description of the microscale texture, the microscale texture may include a porous texture. The method may further comprise machining the microscale texture including the porous texture using a laser ablation process. The method may further comprise coating the microscale texture with a substance at least partially filling the porous texture. The substance may be a resin and/or may be cementum based. The substance may include at least traces of natural or denaturized mammalian dentin. The dentin may be harvested from the tooth of the pre-identified patient. The substance may include a mineral or a mineral aggregate.

Without limiting the foregoing and unless the context requires otherwise, all features or other elements as disclosed herein with respect to FIG. 14 shall be deemed herewith disclosed in any combination with each other, in any partial combination with each other, and in any order; and shall be deemed herewith disclosed in combination with all features or other elements as disclosed herein with respect to FIGS. 1 to 13 in any combination, in any partial combination, and in any order. Without limiting the foregoing and unless the context requires otherwise, the implant or the dental implant referenced with respect to FIG. 14 may represent the same or similar implant, or dental implant, e.g., 100, or 150, 1090, the same or similar workpiece, e.g., 310, 520, 620, 755, 822, 922, or the same and similar like elements, respectively, as shown in and/or described with respect to FIGS. 1, 3, 5 to 9, 10, and other figures herein, respectively, and vice versa. Without limiting the foregoing and unless the context requires otherwise, a macroscale shape as referenced with respect to FIG. 14 may include macroscale shapes, e.g., 110, 112, 114, 116, 160, 161, 164, 166, 170, 400, or 622, as shown in and/or described with respect to FIGS. 1, 4, and other figures herein, respectively, and vice versa; a microscale texture as referenced with respect to FIG. 14 may include microscale textures, e.g., 210, 622, 400, 866, and/or porous surface elements, e.g., 322 as shown in and/or described with respect to FIGS. 2 to 4, 8, and other figures herein, respectively, and vice versa; and a nanoscale topography as referenced with respect to FIG. 14 may include one or more nanoscale surface topographies, e.g., 250, 260, 270, 322, 400, 866, or 924, as shown in and/or described with respect to FIGS. 2 to 4, 8, and 9, respectively, and vice versa. Without limiting the foregoing and unless the context requires otherwise, the implant surface, shape, texture, and/or topography, as applicable, and as referenced with respect to FIG. 14 may represent the same or similar surfaces, shape, texture, and/or topography, as applicable, e.g., 110, 112, 114, 116, 120, 160, 161, 164, 166, 170, 210, 400, 826, 868, or 924, or like elements as shown in and/or described with respect to FIGS. 1 to 4, 8, 9, and other figures herein, respectively. Without limiting the foregoing and unless the context requires otherwise, the anatomical structures, the image data, the clinical data, the patient, the pre-identified patient, the computer numerical control (CNC) data, the computer data or computer data signals, the manufacturing methods or processes, the manufacturing systems, the laser processes, the laser systems, the specification, the machine equipment, as each referenced with respect to FIG. 14 may represent the same or similar anatomical structures, the same or similar image data, the same or similar clinical data, e.g., 1024, 1025, the same or similar patient, e.g., 1010, the same or similar pre-identified patient, e.g., 1010, the same or similar computer numerical control (CNC) data, e.g., 1070, the same or similar computer data or computer data signals, e.g., 1024, 1032, 1059, 1069, and 1070, same or similar the manufacturing methods or processes, e.g., 330, 332, 500, 700, 534, 720, 730, 740, 750, 760, and 1080, the same or similar manufacturing systems, e.g., 1080, the same or similar laser processes, e.g., 300, 540, 600, 612, 930, 950, 979, and 1080, the same or similar laser systems, e.g., 500, and 770, the same or similar specification, e.g., 1090, the same or similar machine equipment, e.g., 1080, or the same and similar like elements, respectively, as shown in and/or described with respect to FIGS. 1, to 3, 5 to 10, 14, and other figures herein, respectively, and vice versa. Without limiting the foregoing and unless the context requires otherwise, the method steps 1410, 1420, 1430, 1440, 1450, or 1460 referenced with respect to FIG. 14 may represent the same or similar method steps, or the same or similar like elements, respectively, as described and/or shown with respect to FIGS. 1 to 13, respectively, and vice versa.

It is to be understood that the specific order or hierarchy of operations in the methods or flow charts depicted in FIGS. 10 to 14 and throughout this disclosure are instances of example approaches and can be rearranged while remaining within the disclosed subject matter. For instance, any of the operations depicted in FIGS. 10 to 14 and throughout this disclosure can be omitted, repeated, performed in parallel, performed in a different order, and/or combined with any other of the operations depicted in FIGS. 10 and 14 and throughout this disclosure.

All the aforementioned embodiments and features and method steps disclosed herein are deemed to be disclosed alone or in any combination, in the disclosed or in reverse order, or in any order as a person skilled in the art would combine and/or order the embodiments, configurations and features and method steps disclosed herein.

It should be understood that one of ordinary skill in the art should understand that the various aspects of the present disclosed technology, as explained above, can readily be combined with each other.

All the aforementioned listed objects, configurations, features, and steps disclosed herein are deemed to include like elements as a person skilled in the art would add elements to those lists.

One of ordinary skill in the art should understand that the various aspects of the presently disclosed technology, as explained above, can readily be combined with each other.

The words used in this disclosure to describe the various exemplary embodiments of the presently disclosed technology are to be understood not only in the sense of their commonly defined meanings, but to include by special definition in this disclosure structure, material, or acts beyond the scope of the commonly defined meanings.

Any enumerations of elements herein shall not be deemed conclusive. It should not be assumed that two or more elements in an enumeration are alternative elements. Two or more elements in an enumeration may describe a similar or the same embodiment. One element in an enumeration may be inclusive of another element in the same enumeration.

The various embodiments of the presently disclosed technology and aspects of embodiments of the technology disclosed herein are to be understood not only in the order and context specifically described in this disclosure, but to include any order and any combination thereof. Whenever the context requires, all words used in the singular number shall be deemed to include the plural and vice versa. Words which import one gender shall be applied to any gender wherever appropriate. Whenever the context requires, all options that are listed with the word “and” shall be deemed to include the world “or” and vice versa, and any combination thereof. If applicable, the words “vice versa” shall be deemed to include the term “the other way around.” Unless the context herein otherwise requires, the words “include”, “for example”, “by way of example”, “exempli gratia” or “e.g.” and derivative or similar terms shall be deemed in each case to be followed by the words “without limitation.” The titles of the sections of this specification and the sectioning of the text in separated paragraphs are for convenience of reference only and are not to be considered in construing this specification.

The words used in this disclosure to describe the various exemplary embodiments of the presently disclosed technology are to be understood not only in the sense of their commonly defined meanings, but to include by special definition in this disclosure structure, material or acts beyond the scope of the commonly defined meanings. Thus, if an element can be understood in the context of this disclosure as including more than one meaning, then its use in a claim must be understood as being generic to all possible meanings supported by the specification and by the word, itself.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

In respect to industrial applicability, it is stated that all embodiments of this disclosure can be applied to an implant, including, without limitation, dental implants, methods, and systems of designing and manufacturing an implant, without limitation dental implants, and custom shaped implants.

What is claimed is:

1. A method to manufacture a customized dental implant for a pre-identified patient, the method comprising:
obtaining a proposed specification of a dental implant, the dental implant including an endosseous root portion and an occlusal facing portion configured to receive a dental prosthesis;
obtaining a trained shape model, the trained shape model being descriptive of a statistical dental anatomy model, the statistical dental anatomy model including one or more statistical dental anatomy element shape models;
obtaining a data set including one or more virtual representations of one or more dental anatomy elements of a dentition of a patient;
forming an adapted shape model based on at least a portion of the trained shape model to fit the one or more virtual representations; and
generating an updated specification by updating the proposed specification of the dental implant based at least in part on the adapted shape model.

2. The method of claim 1, further comprising:
machining the dental implant at least partially based on the updated specification so that a surface of the dental implant at least partially correlates to the adapted shape model.

3. The method of claim 1,
wherein,
the data set includes one or more two-dimensional images representative of the one or more dental anatomy elements of the dentition of the patient, a two-dimensional image of the one or more two-dimensional images includes a plurality of pixels having assigned gradual intensity values, and
the two-dimensional image of the one or more two-dimensional images is a video frame, a picture, a two-dimensional image generated by an intraoral scanner, a two-dimensional array, or an X-ray image.

4. The method of claim 1,
wherein,
the data set includes one or more two-dimensional images representative of the one or more dental anatomy elements of the dentition of the patient,
the one or more two-dimensional images includes a two-dimensional image, and
the two-dimensional image of the one or more two-dimensional images is a two-dimensional point cloud, a two-dimensional mesh, or a two-dimensional shape model.

5. The method of claim 1,
wherein,
the data set includes at least one three-dimensional image of the one or more dental anatomy elements of the dentition of the patient,
the at least one three-dimensional image includes a plurality of voxels having assigned gradual intensity values, and
the at least one three-dimensional image is a CT, a cone beam CT, an MRI image, a three-dimensional X-ray, a frame of a dynamic three-dimensional model, a three-dimensional frame generated by an intraoral scanner, or a three-dimensional array.

6. The method of claim 1,
wherein,
the data set includes at least one three-dimensional image of the one or more dental anatomy elements of the dentition of the patient, and
the at least one three-dimensional image is a three-dimensional point cloud, a three-dimensional mesh, a three-dimensional surface scan, or a three-dimensional shape model.

7. The method of claim 1,
wherein,
the one or more virtual representations of the one or more dental anatomy elements embodied in the data set are unlabeled.

8. The method of claim 1,
wherein,
the adapted shape model includes at least one labeled virtual dental anatomy shape element, and
the at least one labeled virtual dental anatomy shape element includes a numerical three-dimensional surface reconstruction of a corresponding dental anatomy element of the one or more dental anatomy elements.

9. The method of claim 8,
wherein,
the one or more dental anatomy elements includes at least one of a tooth, a portion of the tooth, an alveolar socket, a portion of the alveolar socket, a gingival margin, or a portion of the gingival margin, and
the at least one labeled virtual dental anatomy shape element is associated with a reference to a label corresponding to a dental tooth numbering scheme.

10. The method of claim 1,
wherein,
the one or more statistical dental anatomy element shape models includes a plurality of trained constraint models of virtual statistical shape variabilities.

11. The method of claim 10,
wherein,
the method includes an iterative numerical optimization process having one or more steps including:
varying a virtual size of a virtual shape of a statistical dental anatomy element shape model of the one or more statistical dental anatomy element shape models within at least one virtual size constraint included in the plurality of trained constraint models of virtual statistical shape variabilities,
varying a virtual local deformation of a virtual shape of the statistical dental anatomy element shape model within at least one virtual deformation constraint included in the plurality of trained constraint models of virtual statistical shape variabilities, or
calculating a quality function.

12. The method of claim 1,
wherein,
the updated specification includes at least one virtual three-dimensional design model representing at least a portion of the dental implant selected from a group including at least two of an abutment portion, an occlusal portion, a preparation post to receive a crown, a preparation post to receive a bridge, a preparation post to receive a prosthetic element, a transgingival portion, an implant neck, an endosseous portion, a root portion, an interface between the abutment portion and the endosseous portion, or a root-analogue portion.

13. The method of claim 1,
wherein,
the trained shape model is a multi-dimensional parametrized model,
the one or more statistical dental anatomy element shape models includes a statistical dental anatomy element shape model, and
the statistical dental anatomy element shape model, or forming the adapted shape model, uses a numerical structure including at least one of a point distribution model, a principal component analysis, a vector array, a two-dimensional point cloud, a two-dimensional surface mesh, or a three-dimensional surface mesh.

14. A computer program stored or storable on a non-transitory processor-readable memory as executable instructions which, when executed by one or more processors, performs a computer process comprising:
obtaining a proposed specification of a dental implant, the dental implant including an endosseous root portion and an occlusal facing portion configured to receive a dental prosthesis;
obtaining a trained shape model, the trained shape model being descriptive of a statistical dental anatomy model, the statistical dental anatomy model including one or more statistical dental anatomy element shape models;
obtaining a data set including one or more virtual representations of one or more dental anatomy elements of a dentition of a patient;
forming an adapted shape model based on at least a portion of the trained shape model fitting the one or more virtual representations; and
generating an updated specification by updating the proposed specification of the dental implant based at least in part on the adapted shape model.

15. The computer program of claim 14,
wherein,
the computer process includes visualizing, at a display of an electronic device, an image output of the computer process.

16. The computer program of claim 14,
wherein,
the computer process includes performing a method of teaching the trained shape model.

17. A method to manufacture a customized dental implant for a pre-identified patient, the method comprising:
obtaining a proposed specification of a dental implant, the dental implant includes an endosseous root portion and an occlusal facing portion operable to receive a dental prosthesis;
obtaining a trained coupled shape model, the trained coupled shape model being descriptive of a statistical dental anatomy model, the statistical dental anatomy model including a plurality of labeled statistical dental anatomy element shape models and a plurality of corresponding statistical dental anatomy element orientation models;
obtaining a data set including one or more virtual representations of a plurality of dental anatomy elements of a dentition of a patient;
forming an adapted coupled shape model based on at least a portion of the trained coupled shape model fitting the one or more virtual representations; and
generating an updated specification by updating the proposed specification of the dental implant based at least in part on the adapted coupled shape model.

18. The method of claim 17, further comprising:
machining the dental implant based at least in part on the updated specification so that a surface of the dental implant at least partially correlates to the adapted coupled shape model.

19. The method of claim 17,
wherein,
the data set includes one or more two-dimensional images representative of the plurality of dental anatomy elements of the dentition of the patient, a two-dimensional image of the one or more two-dimensional images including a plurality of pixels having assigned gradual intensity values; and
the two-dimensional image of the one or more two-dimensional images is a video frame, a picture, a two-dimensional image generated by an intraoral scanner, a two-dimensional array, or an X-ray image.

20. The method of claim 17,
wherein,
the data set includes at least one two-dimensional image representative of the plurality of dental anatomy elements of the dentition of the patient, and
the at least one two-dimensional image is a two-dimensional point cloud, a two-dimensional mesh, a two-dimensional shape model, or a two-dimensional coupled shape model.

21. The method of claim 17,
wherein,
the data set includes at least one three-dimensional image of the plurality of dental anatomy elements of the dentition of the patient,
the at least one three-dimensional image includes a plurality of voxels having assigned gradual intensity values, and
the at least one three-dimensional image is a CT, a cone beam CT, an MRI image, a three-dimensional X-ray, a frame of a dynamic three-dimensional model, a three-dimensional frame generated by an intraoral scanner, or a three-dimensional array.

22. The method of claim 17,
wherein,
the data set includes at least one three-dimensional image of the plurality of dental anatomy elements of the dentition of the patient, and
the at least one three-dimensional image is a three-dimensional point cloud, a three-dimensional mesh, a three-dimensional surface scan, or a three-dimensional coupled shape model.

23. The method of claim 17,
wherein,
the one or more virtual representations of the plurality of dental anatomy elements embodied in the data set are unlabeled.

24. The method of claim 17,
wherein,
the adapted coupled shape model includes at least one labeled virtual dental anatomy shape element, and
the at least one labeled virtual dental anatomy shape element includes a numerical three-dimensional surface reconstruction of a corresponding dental anatomy element of the plurality of dental anatomy elements.

25. The method of claim 24,
wherein,
the plurality of dental anatomy elements includes at least one of a tooth, a portion of the tooth, an alveolar socket, a portion of the alveolar socket, a gingival margin, or a portion of the gingival margin, and
the at least one labeled virtual dental anatomy shape element is associated with a reference to a label corresponding to a dental tooth numbering scheme.

26. The method of claim 17,
wherein,
at least one labeled statistical dental anatomy element shape model of the plurality of labeled statistical dental anatomy element shape models includes a plurality of trained shape constraint models of virtual statistical shape variabilities, and
the plurality of corresponding statistical dental anatomy element orientation models includes a trained orientation constraint model of virtual statistical orientation variability.

27. The method of claim 26,
wherein,
the forming includes performing an iterative numerical optimization process having one or more steps including:
varying a virtual size of a virtual shape of a labeled statistical dental anatomy element shape model of the plurality of labeled statistical dental anatomy element shape models within at least one virtual size constraint included in the plurality of trained shape constraint models of virtual statistical shape variabilities,
varying a virtual local deformation of a virtual shape of the labeled statistical dental anatomy element shape model within at least one virtual deformation constraint included in the plurality of trained shape constraint models of virtual statistical shape variabilities, or
varying a virtual orientation of a virtual shape of the labeled statistical dental anatomy element shape model within at least one virtual orientation constraint included in the trained orientation constraint model of virtual statistical orientation variability,
and
the iterative numerical optimization process includes calculating a quality function.

28. The method of claim 17,
wherein,
the updated specification includes at least one virtual three-dimensional design model representing at least a portion of the dental implant including at least one of an abutment portion, an occlusal portion, a preparation post to receive a crown, a preparation post to receive a bridge, a preparation post to receive a prosthetic element, a transgingival portion, an implant neck, an endosseous portion, a root portion, an interface between the abutment portion and the endosseous portion, or a root-analogue portion.

29. The method of claim 17,
wherein,
the trained coupled shape model is a multi-dimensional parametrized model including at least one of a static two-dimensional model, a dynamic two-dimensional model, a three-dimensional model, or a dynamic three-dimensional model, and
the plurality of labeled statistical dental anatomy element shape models, or forming the adapted coupled shape model, uses at least one numerical structure being at least one of a point distribution model, a principal component analysis, a vector array, a two-dimensional point cloud, a two-dimensional surface mesh, or a three-dimensional surface mesh.

30. A computer program stored or storable on a non-transitory processor-readable memory as executable instructions which, when executed by one or more processors, performs a computer process comprising:
obtaining a proposed specification of a dental implant, the dental implant includes an endosseous root portion and an occlusal facing portion operable to receive a dental prosthesis;
obtaining a trained coupled shape model, the trained coupled shape model being descriptive of a statistical dental anatomy model, the statistical dental anatomy model including a plurality of labeled statistical dental anatomy element shape models and a plurality of corresponding statistical dental anatomy element orientation models;
obtaining a data set including one or more virtual representations of a plurality of dental anatomy elements of a dentition of a patient;
forming an adapted coupled shape model based on at least a portion of the trained coupled shape model to fit the one or more virtual representations; and
generating an updated specification by updating the proposed specification of the dental implant based at least in part on the adapted coupled shape model.

31. The computer program of claim 30,
wherein,
the computer process includes visualizing, at display of an electronic device, an image output of the computer process.

32. The computer program of claim 30,
wherein,
the computer process includes performing a method of teaching the trained coupled shape model.

33. A method to teach a dental anatomy machine learning model, the method comprising:
obtaining one or more individual exemplary dental anatomy models descriptive of one or more individual exemplary virtual dental anatomy shape elements;
obtaining a trainable or trained shape model, the trainable or trained shape model is descriptive of a statistical dental anatomy model, the statistical dental anatomy model includes one or more statistical dental anatomy element shape models; and
generating an updated trained shape model by updating the trainable or trained shape model based at least in part on the one or more individual exemplary dental anatomy models.

34. The method of claim 33,
wherein,
the one or more statistical dental anatomy element shape models includes a plurality of corresponding trained constraint models of virtual statistical shape variabilities, and
the updating of the trainable or trained shape model includes updating, for the one or more statistical dental anatomy element shape models, the plurality of corresponding trained constraint models of virtual statistical shape variabilities based at least in part on a shape variability of the one or more individual exemplary virtual dental anatomy shape elements.

35. A method to teach a dental anatomy machine learning model, the method comprising:
obtaining one or more individual exemplary dental anatomy models descriptive of a plurality of individual exemplary labeled virtual dental anatomy shape elements and corresponding exemplary virtual relative orientations;
obtaining a trainable or trained coupled shape model, the trainable or trained coupled shape model being descriptive of a statistical dental anatomy model, the statistical dental anatomy model including a plurality of labeled statistical dental anatomy element shape models and a plurality of corresponding statistical orientation models; and
generating an updated trained coupled shape model by updating the trainable or trained coupled shape model based at least in part on the one or more individual exemplary dental anatomy models.

36. The method of claim 35,
wherein,
the plurality of labeled statistical dental anatomy element shape models includes a plurality of corresponding trained shape constraint models of virtual statistical shape variabilities, and
the updating of the trainable or trained coupled shape model includes updating, for the plurality of labeled statistical dental anatomy element shape models, the plurality of corresponding trained shape constraint models of virtual statistical shape variabilities based at least in part on a shape variability of the plurality of individual exemplary labeled virtual dental anatomy shape elements.

37. The method of claim 35,
wherein,
a corresponding statistical orientation models of the plurality of corresponding statistical orientation models includes a trained orientation constraint model of virtual statistical orientation variability, and
the updating of the trainable or trained coupled shape model includes updating, for a labeled statistical dental anatomy element shape model of the plurality of labeled statistical dental anatomy element shape models, the trained orientation constraint model of virtual statistical orientation variability based at least in part on an orientation variability of the plurality of corresponding statistical orientation models.
